(19) United States
(12) Patent Application Publication
(10) Pub. No.: US 2025/0213143 A1
(43) Pub. Date: Jul. 3, 2025

(54) OVERMOLDED STRUCTURES FOR ANALYTE SENSORS

(71) Applicant: DexCom, Inc., San Diego, CA (US)

(72) Inventors: John Paul Rukavina, San Diego, CA (US); Carl E. Hoffmeier, San Diego, CA (US); Nicolas Robert Medjo, San Diego, CA (US); William Eric Reyna, San Diego, CA (US); Zachary Felix Wood, San Diego, CA (US); Jamison Scott Woodward, San Diego, CA (US)

(21) Appl. No.: 19/004,016

(22) Filed: Dec. 27, 2024

Related U.S. Application Data
(60) Provisional application No. 63/616,251, filed on Dec. 29, 2023.

(51) Int. Cl. A61B 5/145

(52) U.S. Cl. CPC A61B 5/14503; A61B 5/14532; A61B 5/14546

(57) ABSTRACT

Implementations relate generally to devices for measuring an analyte in a host. Implementations may provide bodies overmolded upon at least a portion of an elongate sensor. The bodies may comprise sensor modules for mechanical and/or electrical connection with other structures, which may comprise sensor electronics or testing apparatuses for the elongate sensor.

### CROSS-REFERENCE TO RELATED APPLICATIONS

This application claims the benefit of U.S. Provisional Application No. 63/616,251, filed Dec. 29, 2023, the entire contents of which are incorporated by reference herein.

### BACKGROUND

### Field

Systems, methods, and devices for measuring an analyte in an individual are provided. More particularly, on-skin wearable medical devices that are wearable by a host and having an analyte sensor are provided.

### Description of the Related Technology

Diabetes mellitus is a disorder in which the pancreas cannot create sufficient insulin (Type I or insulin dependent) and/or in which insulin is not effective (Type 2 or non-insulin dependent). In the diabetic state, the victim suffers from high blood sugar, which can cause an array of physiological derangements associated with the deterioration of small blood vessels, for example, kidney failure, skin ulcers, or bleeding into the vitreous of the eye. A hypoglycemic reaction (low blood sugar) can be induced by an inadvertent overdose of insulin, or after a normal dose of insulin or glucose-lowering agent accompanied by extraordinary exercise or insufficient food intake.

Conventionally, a person with diabetes carries a self-monitoring blood glucose (SMBG) monitor, which typically requires uncomfortable finger pricking methods. Due to the lack of comfort and convenience, a person with diabetes normally only measures his or her glucose levels two to four times per day. Unfortunately, such time intervals are spread so far apart that the person with diabetes likely finds out too late of a hyperglycemic or hypoglycemic condition, sometimes incurring dangerous side effects. Glucose levels may be alternatively monitored continuously by a measurement system including an on-skin sensor assembly. The sensor assembly may have a wireless transmitter which transmits measurement data to a receiver which can process and display information based on the measurements.

Construction, testing, assembly, and handing of the analyte sensors may be difficult due to the small size and fragile nature of the analyte sensors. Care must be taken to avoid damage to the analyte sensor during testing and general handling of the analyte sensors.

This Background is provided to introduce a brief context for the Summary and Detailed Description that follow. This Background is not intended to be an aid in determining the scope of the claimed subject matter nor be viewed as limiting the claimed subject matter to implementations that solve any or all of the disadvantages or problems presented above.

### SUMMARY

The present systems and methods relate to apparatuses, systems, and methods for medical devices. More particularly, apparatuses, systems, and methods are provided for sensors for medical devices, which may include a transcutaneous analyte sensor for deployment to the skin of a host. The various examples of the present apparatuses, systems, and methods may have several features, no single one of which is solely responsible for their desirable attributes. Without limiting the scope of the present examples as expressed by the claims that follow, their more prominent features now will be discussed briefly. After considering this discussion, and particularly after reading the section entitled “Detailed Description,” one will understand how the features of the present examples provide the advantages described herein.

In a first aspect, a sensor module comprising: an elongate sensor having a distal end portion and a proximal end portion, the distal end portion configured to be inserted into skin of a host and the proximal end portion configured to be positioned exterior of the skin of the host; and a body molded upon at least a portion of the elongate sensor.

Implementations of the embodiments may include one or more of the following. The elongate sensor may include an outer surface, and the body forms a seal upon at least a portion of the outer surface of the elongate sensor. The body may comprise a multi-shot overmolding upon at least the portion of the elongate sensor. The body may comprise a frame disposed at the proximal end portion of the elongate sensor. The proximal end portion of the elongate sensor may include one or more sensor electrical contacts, the one or more sensor electrical contacts being positioned within an interior chamber of the frame. The frame may include one or more openings configured to allow for electrical connection of the one or more sensor electrical contacts with one or more electrical contacts of a sensor electronics module. The frame may include an upper outer surface and a lower outer surface facing opposite the upper outer surface, the upper outer surface and the lower outer surface each extending along a length of the proximal end portion of the elongate sensor, and the one or more openings being positioned in the upper outer surface or the lower outer surface. The one or more sensor electrical contacts may include a first sensor electrical contact and a second sensor electrical contact, and the interior chamber is a first interior chamber, and the frame includes a second interior chamber, and the first sensor electrical contact is positioned within the first interior chamber, and the second sensor electrical contact is positioned within the second interior chamber. The first interior chamber may be spaced from the second interior chamber in a longitudinal direction along a length of the proximal end portion of the elongate sensor. The first interior chamber may be spaced from the second interior chamber in a lateral direction that is lateral relative to a length of the proximal end portion of the elongate sensor. The first interior chamber and the second interior chamber may each be sub-chambers of an interior chamber of the frame. The frame may have a larger cross-sectional dimension than a cross-sectional dimension of the proximal end portion of the elongate sensor and includes one or more handling surfaces. The frame may have a substantially rectangular outer profile. The frame may include at least one mating feature configured to mate the frame with a corresponding structure of a wearable device. The body may comprise one or more electrical contacts, with the frame comprising a first material and the one or more electrical contacts of the body comprising a second material different from the first material. The one or more electrical contacts of the body may be positioned within an interior chamber of the frame. The frame may comprise a rigid thermoplastic material. The body may comprise one or more electrical contacts. The one or more electrical contacts of the body may comprise a conductive elastomeric material. The proximal end portion of the elongate sensor may include one or more sensor electrical contacts, and each of the one or more electrical contacts of the body surrounds a corresponding one of the one or more sensor electrical contacts. The one or more electrical contacts of the body may be configured for a surface-to-surface connection with one or more electrical contacts of a sensor electronics module. The one or more electrical contacts of the body may be configured to be penetrated by one or more electrical contacts of a sensor electronics module. The body may comprise a frame disposed at the proximal end portion of the elongate sensor, the frame including an outer surface, and the one or more electrical contacts of the body protrude from the outer surface. The body may include a sealing member configured to seal an electrical connection between the one or more electrical contacts of the body and one or more electrical contacts of a sensor electronics module. The sealing member may include one or more ribs or grooves. The sealing member may cover the one or more electrical contacts of the body and is configured to be penetrated by one or more external electrical contacts of a sensor electronics module. The sealing member may form an outer surface of the body, and the one or more electrical contacts of the body protrude from the outer surface. The body may comprise a frame disposed at the proximal end portion of the elongate sensor, with the frame comprising a first material and the one or more electrical contacts of the body comprising a second material different from the first material, and the sealing member comprises a third material, the third material being different from the first material. The body may comprise a frame disposed at the proximal end portion of the elongate sensor and having an interior chamber, and the sealing member fills the interior chamber of the frame. The body may be molded upon an outer surface of the proximal end portion of the elongate sensor, and the distal end portion of the elongate sensor is uncovered by the body. The distal end portion of the elongate sensor may protrude from the body. The body may encapsulate the proximal end portion of the elongate sensor. The elongate sensor may comprise a coaxial wire. The body may include a sealing member configured to form a radial seal with a socket configured to receive the body. The sealing member may include one or more ribs or grooves extending circumferentially about an outer surface of the body. The body may include a flange protruding radially outward from the body and extending circumferentially about the body. The flange may be configured for bonding with at least a portion of a socket configured to receive the body. The elongate sensor may comprise a planar sensor. The proximal end portion of the elongate sensor may include a planar substrate. The planar substrate may include one or more openings. The planar substrate may include one or more sensor electrical contacts, the one or more sensor electrical contacts including at least one of the one or more openings. The one or more openings of the one or more sensor electrical contacts may be configured to receive an electrical contact for electrical connection with a respective one of the sensor electrical contacts. The one or more openings may be configured for receiving material of the body for securing the planar substrate to the body. The distal end portion of the elongate sensor may comprise an insertion portion of the elongate sensor, the insertion portion being planar and having a thickness and a width that is greater than the thickness, the insertion portion being configured to bend about an axis that extends in a dimension that the width extends along. The planar substrate may extend in the dimension, and the planar substrate is configured to receive one or more electrical contacts therethrough in a direction transverse to the dimension. The distal end portion of the elongate sensor may comprise an insertion portion of the elongate sensor, the insertion portion being planar and having a thickness and a width that is greater than the thickness, the insertion portion being configured to bend about an axis that extends transverse to a dimension that the width extends along. The planar substrate may extend in the dimension and includes one or more sensor electrical contacts, and the body includes at least one electrical contact configured to contact the one or more sensor electrical contacts in a direction that is transverse to the dimension. The planar substrate may include a plurality of sensor electrical contacts spaced from each other in a longitudinal direction along a length of the planar substrate. The planar substrate may include a plurality of sensor electrical contacts spaced from each other in a lateral direction that is lateral relative to a length of the planar substrate. The elongate sensor may be configured to detect a first analyte and a second analyte that is different than the first analyte and includes one or more sensor electrical contacts for providing an electrical signal corresponding to the first analyte and to the second analyte. The proximal end portion of the elongate sensor may include a planar substrate having the one or more sensor electrical contacts. The elongate sensor may be a bent elongate sensor. The elongate sensor may be a transcutaneous analyte sensor. The sensor module may be configured to electrically connect with electronics for receiving an electrical signal from the elongate sensor. The elongate sensor may be configured to detect glucose within extracellular fluid of the host. The body may be physically bonded to at least the portion of the elongate sensor.

In a second aspect, an on-skin wearable medical device comprising: a wearable housing for wear on skin of a host; an elongate sensor having a distal end portion and a proximal end portion, the distal end portion configured to be inserted into the skin of the host and the proximal end portion configured to be positioned exterior of the skin of the host; a body molded upon at least a portion of the elongate sensor, the body being configured to be coupled to the wearable housing; and a sensor electronics module operably coupled to the wearable housing and configured to receive an electrical signal from the elongate sensor.

Implementations of the embodiments may include one or more of the following. A socket may be coupled to the wearable housing and configured to receive the body. The socket may include one or more mating features configured to mate with the body. The sensor electronics module may comprise one or more electrical contacts configured to electrically connect with one or more corresponding electrical contacts of the body, at least when the body is received by the socket. A circuit board substrate may be positioned within the wearable housing, and wherein the socket is positioned upon the circuit board substrate. The socket may be formed upon the circuit board substrate with surface mount technology. The socket may be formed in a wall of the wearable housing. The body may be molded upon the proximal end portion of the elongate sensor, and the socket is configured to receive the body in a direction transverse to a length of the proximal end portion of the elongate sensor. The body may be a sensor module configured to mate with the socket. The on-skin wearable medical device may be a first on-skin wearable medical device, and the sensor module is configured for insertion in a socket of a second on-skin wearable medical device having a different configuration than the first on-skin wearable medical device. The sensor module may be configured for coupling with a testing apparatus for the elongate sensor. The elongate sensor may include an outer surface, and the body forms a seal upon at least a portion of the outer surface of the elongate sensor. The body may comprise a multi-shot overmolding upon at least the portion of the elongate sensor. The body may comprise a frame disposed at the proximal end portion of the elongate sensor. The proximal end portion of the elongate sensor may include one or more sensor electrical contacts, the one or more sensor electrical contacts being positioned within an interior chamber of the frame. An electrical connection between the one or more sensor electrical contacts and the one or more electrical contacts of the sensor electronics module may comprise a solidified conductive liquid. The frame may include one or more openings configured to allow for electrical connection of the one or more sensor electrical contacts with one or more electrical contacts of the sensor electronics module. The frame may include an upper outer surface and a lower outer surface facing opposite the upper outer surface, the upper outer surface and the lower outer surface each extending along a length of the proximal end portion of the elongate sensor, and the one or more openings being positioned in the upper outer surface or the lower outer surface. The one or more sensor electrical contacts may include a first sensor electrical contact and a second sensor electrical contact, and the interior chamber is a first interior chamber, and the frame includes a second interior chamber, and the first sensor electrical contact is positioned within the first interior chamber, and the second sensor electrical contact is positioned within the second interior chamber. The first interior chamber may be spaced from the second interior chamber in a longitudinal direction along a length of the proximal end portion of the elongate sensor. The first interior chamber may be spaced from the second interior chamber in a lateral direction that is lateral relative to a length of the proximal end portion of the elongate sensor. The first interior chamber and the second interior chamber may each be sub-chambers of an interior chamber of the frame. The frame may have a larger cross-sectional dimension than a cross-sectional dimension of the proximal end portion of the elongate sensor and includes one or more handling surfaces. The frame may have a substantially rectangular outer profile. The frame may include at least one mating feature configured to mate the frame with a corresponding structure of the wearable housing. The body may comprise one or more electrical contacts, with the frame comprising a first material and the one or more electrical contacts of the body comprising a second material different from the first material. The one or more electrical contacts of the body may be positioned within an interior chamber of the frame. The frame may comprise a rigid thermoplastic material. The body may comprise one or more electrical contacts. The one or more electrical contacts of the body may comprise a conductive elastomeric material. A circuit board substrate may be positioned within the wearable housing, and wherein the one or more electrical contacts of the body are electrically coupled to one or more electrical contacts that are positioned on the circuit board substrate. The proximal end portion of the elongate sensor may include one or more sensor electrical contacts, and each of the one or more electrical contacts of the body surrounds a corresponding one of the one or more sensor electrical contacts. The one or more electrical contacts of the body may be configured for a surface-to-surface connection with one or more electrical contacts of the sensor electronics module. The one or more electrical contacts of the body may be configured to be penetrated by one or more electrical contacts of the sensor electronics module. The body may comprise a frame disposed at the proximal end portion of the elongate sensor, the frame including an outer surface, and the one or more electrical contacts of the body protrude from the outer surface. The body may include a sealing member configured to seal an electrical connection between the one or more electrical contacts of the body and one or more electrical contacts of the sensor electronics module. The sealing member may include one or more ribs or grooves. The sealing member may cover the one or more electrical contacts of the body and is configured to be penetrated by one or more external electrical contacts of the sensor electronics module. The sealing member may form an outer surface of the body, and the one or more electrical contacts of the body protrude from the outer surface. The body may comprise a frame disposed at the proximal end portion of the elongate sensor, with the frame comprising a first material and the one or more electrical contacts of the body comprising a second material different from the first material, and the sealing member comprises a third material, the third material being different from the first material. The body may comprise a frame disposed at the proximal end portion of the elongate sensor and having an interior chamber, and the sealing member fills the interior chamber of the frame. The body may be molded upon an outer surface of the proximal end portion of the elongate sensor, and the distal end portion of the elongate sensor is uncovered by the body. The distal end portion of the elongate sensor may protrude from the body. The body may encapsulate the proximal end portion of the elongate sensor. The elongate sensor may comprise a coaxial wire. The body may include a sealing member configured to form a radial seal with a socket configured to receive the body. The body may include a flange protruding radially outward from the body and extending circumferentially about the body. The elongate sensor may comprise a planar sensor. The proximal end portion of the elongate sensor may include a planar substrate. The planar substrate may include one or more openings. The planar substrate may include one or more sensor electrical contacts, the one or more sensor electrical contacts including at least one of the one or more openings. The one or more openings of the one or more sensor electrical contacts may be configured to receive an electrical contact for electrical connection with a respective one of the sensor electrical contacts. The one or more openings may be configured for receiving material of the body for securing the planar substrate to the body. The distal end portion of the elongate sensor may comprise an insertion portion of the elongate sensor, the insertion portion being planar and having a thickness and a width that is greater than the thickness, the insertion portion being configured to bend about an axis that extends in a dimension that the width extends along. The planar substrate may extend in the dimension, and the planar substrate is configured to receive one or more electrical contacts therethrough in a direction transverse to the dimension. The distal end portion of the elongate sensor may comprise an insertion portion of the elongate sensor, the insertion portion being planar and having a thickness and a width that is greater than the thickness, the insertion portion being configured to bend about an axis that extends transverse to a dimension that the width extends along. The planar substrate may extend in the dimension and includes one or more sensor electrical contacts, and the body includes at least one electrical contact configured to contact the one or more sensor electrical contacts in a direction that is transverse to the dimension. The planar substrate may include a plurality of sensor electrical contacts spaced from each other in a longitudinal direction along a length of the planar substrate. The planar substrate may include a plurality of sensor electrical contacts spaced from each other in a lateral direction that is lateral relative to a length of the planar substrate. The elongate sensor may be configured to detect a first analyte and a second analyte that is different than the first analyte and includes one or more sensor electrical contacts for providing an electrical signal corresponding to the first analyte and to the second analyte. The proximal end portion of the elongate sensor may include a planar substrate having the one or more sensor electrical contacts. The elongate sensor may be a bent elongate sensor. The elongate sensor may be a transcutaneous analyte sensor. The body may comprise a sensor module configured to electrically connect with the sensor electronics module for receiving an electrical signal from the elongate sensor. The elongate sensor may be configured to detect glucose within extracellular fluid of the host. The body may be physically bonded to at least the portion of the elongate sensor.

In a third aspect, a method of fabricating at least a portion of an on-skin wearable medical device, the method comprising: providing an elongate sensor having a distal end portion and a proximal end portion, the distal end portion configured to be inserted into skin of a host and the proximal end portion configured to be positioned exterior of the skin of the host; and molding a body upon at least a portion of the elongate sensor.

Implementations of the embodiments may include one or more of the following. The body may comprise a frame disposed at the proximal end portion of the elongate sensor. The proximal end portion of the elongate sensor may include one or more sensor electrical contacts, the one or more sensor electrical contacts being positioned within an interior chamber of the frame. The frame may include one or more openings configured to allow for electrical connection of the one or more sensor electrical contacts with one or more electrical contacts of a sensor electronics module. The one or more sensor electrical contacts may include a first sensor electrical contact and a second sensor electrical contact, and the interior chamber is a first interior chamber, and the frame includes a second interior chamber, and the first sensor electrical contact is positioned within the first interior chamber, and the second sensor electrical contact is positioned within the second interior chamber. The first interior chamber may be spaced from the second interior chamber in a longitudinal direction along a length of the proximal end portion of the elongate sensor. The first interior chamber may be spaced from the second interior chamber in a lateral direction that is lateral relative to a length of the proximal end portion of the elongate sensor. The first interior chamber and the second interior chamber may each be sub-chambers of an interior chamber of the frame. The method may further comprise electrically connecting the one or more sensor electrical contacts with one or more electrical contacts of a sensor electronics module using a cured liquid. The body may comprise one or more electrical contacts, with the frame comprising a first material and the one or more electrical contacts of the body comprising a second material different from the first material. The method may further comprise molding the frame in a first mold, and molding the one or more electrical contacts of the body in a second mold. The body may comprise one or more electrical contacts. The proximal end portion of the elongate sensor may include one or more sensor electrical contacts, and each of the one or more electrical contacts of the body surrounds a corresponding one of the one or more sensor electrical contacts. The one or more electrical contacts of the body may be configured to be penetrated by one or more electrical contacts of a sensor electronics module. The body may include a sealing member configured to seal an electrical connection between the one or more electrical contacts of the body and one or more electrical contacts of a sensor electronics module. The sealing member may cover the one or more electrical contacts of the body and is configured to be penetrated by one or more external electrical contacts of a sensor electronics module. The sealing member may form an outer surface of the body, and the one or more electrical contacts of the body protrude from the outer surface. The body may comprise a frame disposed at the proximal end portion of the elongate sensor, with the frame comprising a first material and the one or more electrical contacts of the body comprising a second material different from the first material, and the sealing member comprises a third material, the third material being different from the first material. The method may further comprise molding the frame in a first mold, and molding the one or more electrical contacts of the body in a second mold, and molding the sealing member in a third mold. The method may further comprise coupling the body to a wearable housing configured to be worn on skin of a host. The method may further comprise inserting the body into a socket configured to receive the body. The method may further comprise electrically coupling the elongate sensor with a sensor electronics module configured to receive an electrical signal from the elongate sensor. The elongate sensor may comprise a coaxial wire. The body may include a sealing member configured to form a radial seal with a socket configured to receive the body. The body may include a flange protruding radially outward from the body and extending circumferentially about the body. The elongate sensor may comprise a planar sensor. The proximal end portion of the elongate sensor may include a planar substrate. The planar substrate may include one or more openings. The planar substrate may include one or more sensor electrical contacts, the one or more sensor electrical contacts including at least one of the one or more openings. The one or more openings of the one or more sensor electrical contacts may be configured to receive an electrical contact for electrical connection with a respective one of the sensor electrical contacts. The one or more openings may be configured for receiving material of the body for securing the planar substrate to the body. The distal end portion of the elongate sensor may comprise an insertion portion of the elongate sensor, the insertion portion being planar and having a thickness and a width that is greater than the thickness, the insertion portion being configured to bend about an axis that extends in a dimension that the width extends along. The planar substrate may extend in the dimension, and the planar substrate is configured to receive one or more electrical contacts therethrough in a direction transverse to the dimension. The distal end portion of the elongate sensor may comprise an insertion portion of the elongate sensor, the insertion portion being planar and having a thickness and a width that is greater than the thickness, the insertion portion being configured to bend about an axis that extends transverse to a dimension that the width extends along. The planar substrate may extend in the dimension and includes one or more sensor electrical contacts, and the body includes at least one electrical contact configured to contact the one or more sensor electrical contacts in a direction that is transverse to the dimension. The planar substrate may include a plurality of sensor electrical contacts spaced from each other in a longitudinal direction along a length of the planar substrate. The planar substrate may include a plurality of sensor electrical contacts spaced from each other in a lateral direction that is lateral relative to a length of the planar substrate. The elongate sensor may be a multi-analyte sensor. The elongate sensor may be a bent elongate sensor. The elongate sensor may be a transcutaneous analyte sensor. The elongate sensor may be configured to detect glucose within extracellular fluid of the host.

In further aspects and embodiments, the above methods and features of the various aspects are formulated in terms of a system as in various aspects, having an applicator configured to carry out the method features. Any of the features of an embodiment of any of the aspects, including but not limited to any embodiments of any of the first through third aspects referred to above, is applicable to all other aspects and embodiments identified herein, including but not limited to any embodiments of any of the first through third aspects referred to above. Moreover, any of the features of an embodiment of the various aspects, including but not limited to any embodiments of any of the first through third aspects referred to above, is independently combinable, partly or wholly with other embodiments described herein in any way, e.g., one, two, or three or more embodiments may be combinable in whole or in part. Further, any of the features of an embodiment of the various aspects, including but not limited to any embodiments of any of the first through third aspects referred to above, may be made optional to other aspects or embodiments. Any aspect or embodiment of a method can be performed by a system or apparatus of another aspect or embodiment, and any aspect or embodiment of a system or apparatus can be configured to perform a method of another aspect or embodiment, including but not limited to any embodiments of any of the first through third aspects referred to above.

This Summary is provided to introduce a selection of concepts in a simplified form. The concepts are further described in the Detailed Description section. Elements or steps other than those described in this Summary are possible, and no element or step is necessarily required. This Summary is not intended to identify key features or essential features of the claimed subject matter, nor is it intended for use as an aid in determining the scope of the claimed subject matter. The claimed subject matter is not limited to implementations that solve any or all disadvantages noted in any part of this disclosure.

### BRIEF DESCRIPTION OF THE DRAWINGS

These and other features, aspects, and advantages are described below with reference to the drawings, which are intended to illustrate, but not to limit, the disclosure. In the drawings, like reference characters denote corresponding features consistently throughout similar embodiments.

FIG. 1 is a schematic view of an on-skin wearable system attached to a host and communicating with a plurality of example devices.

FIG. 2 is a block diagram that illustrates electronics associated with the on-skin wearable system of FIG. 1.

FIGS. 3A-3C illustrate an on-skin wearable medical device having a transcutaneous analyte sensor.

FIG. 3D illustrates a schematic view of an analyte sensor.

FIG. 4 illustrates a perspective view of an analyte sensor system or module.

FIG. 5 illustrates a top view of the analyte sensor system shown in FIG. 4.

FIG. 6 illustrates a bottom view of the analyte sensor system shown in FIG. 4.

FIG. 7 illustrates a cross sectional view of the analyte sensor system shown in FIG. 4.

FIG. 8A illustrates a top perspective view of an on-skin wearable medical device.

FIG. 8B illustrates a bottom perspective view of the on-skin wearable medical device shown in FIG. 8A.

FIG. 8C illustrates an exploded view of the on-skin wearable medical device shown in FIG. 8A.

FIG. 9 illustrates a bottom perspective view of a housing of an on-skin wearable medical device showing insertion of an analyte sensor system.

FIG. 10 illustrates a cross sectional view of the analyte sensor system of FIG. 9 inserted into a socket of the on-skin wearable medical device shown in FIG. 9.

FIG. 11 illustrates a cross sectional view of the analyte sensor system of FIG. 9 inserted into a socket of the on-skin wearable medical device shown in FIG. 9 and encapsulated with a sealing member.

FIG. 12 illustrates a perspective view of an analyte sensor system or module.

FIG. 13 illustrates a perspective view of the electrical contacts of FIG. 12 isolated from the frame of FIG. 12.

FIG. 14 illustrates a top view of the analyte sensor system shown in FIG. 12.

FIG. 15 illustrates a cross sectional view of the analyte sensor system shown in FIG. 12.

FIG. 16 illustrates a side view of an analyte sensor system.

FIG. 17 illustrates a cross sectional view of the analyte sensor system shown in FIG. 12 inserted into a socket.

FIG. 18 illustrates a perspective view of protrusions in the form of pins.

FIG. 19 illustrates a side view of one of the pins shown in FIG. 18.

FIG. 20 illustrates a perspective view of a protrusion in the form of a pin.

FIG. 21 illustrates a perspective view of protrusions in the form of pins.

FIG. 22 illustrates a perspective view of protrusions in the form of pins coupled to a circuit board substrate.

FIG. 23 illustrates a side view of one of the pins shown in FIG. 22.

FIG. 24 illustrates a perspective view of a protrusion in the form of a pin.

FIG. 25 illustrates a bottom perspective view of a housing of an on-skin wearable medical device showing insertion of an analyte sensor system or module.

FIG. 26 illustrates a cross sectional view of the analyte sensor system of FIG. 12 inserted into a socket of the on-skin wearable medical device shown in FIG. 25.

FIG. 27 illustrates a cross sectional view of a mating of the analyte sensor system of FIG. 12 inserted into a socket of the on-skin wearable medical device shown in FIG. 25.

FIG. 28 illustrates a perspective view of an analyte sensor system.

FIG. 29 illustrates a side cross sectional view of the analyte sensor system shown in FIG. 28.

FIG. 30 illustrates a bottom perspective view of the analyte sensor system shown in FIG. 28.

FIG. 31 illustrates a transverse cross sectional view of the analyte sensor system along line I-I shown in FIG. 28.

FIG. 32 illustrates a transverse cross sectional view of the analyte sensor system along line

II-II shown in FIG. 28.

FIG. 33 illustrates a transverse cross sectional view of the analyte sensor system along line III-III shown in FIG. 28.

FIG. 34 illustrates a bottom perspective view of an analyte sensor system.

FIG. 35 illustrates a cross sectional view of the analyte sensor system shown in FIG. 34.

FIG. 36 illustrates a cross sectional view of the analyte sensor system shown in FIG. 34 mated with electrical contacts.

FIG. 37 illustrates a perspective view of a socket of an on-skin wearable device.

FIG. 38 illustrates a perspective view of the socket shown in FIG. 37 mated with an analyte sensor system.

FIG. 39 illustrates a transverse cross sectional view of the socket shown in FIG. 37 mated with the analyte sensor system, as shown in FIG. 38.

FIG. 40 illustrates a perspective view of a socket mated with an analyte sensor system.

FIG. 41A illustrates a bottom perspective view of a socket in a portion of a wearable housing.

FIG. 41B illustrates a top perspective view of a socket shown in FIG. 41A.

FIG. 42A illustrates a top perspective view of a socket in a portion of a wearable housing.

FIG. 42B illustrates a bottom perspective view of the socket shown in FIG. 42A.

FIG. 43 illustrates a top perspective view of a socket mated with an analyte sensor system.

FIG. 44 illustrates a top perspective view of a printed circuit board positioned over the analyte sensor system shown in FIG. 43.

FIG. 45 illustrates a perspective view of a socket, with an analyte sensor system positioned above the socket.

FIG. 46 illustrates a perspective view of a socket.

FIG. 47 illustrates a perspective view of a lead frame for the socket shown in FIG. 46.

FIG. 48 illustrates a top view of the socket shown in FIG. 46.

FIG. 49 illustrates a side cross sectional view of an analyte sensor system positioned within the socket shown in FIG. 46.

FIG. 50 illustrates a perspective view of a socket coupled to a power source carriage.

FIG. 51 illustrates a perspective view of the socket coupled to the power source carriage of FIG. 50 with a respective analyte sensor system and battery coupled thereto.

FIG. 52 illustrates an exploded view of an on-skin wearable device.

FIG. 53 illustrates a partially assembled view of the on-skin wearable device shown in FIG. 52.

FIG. 54 illustrates an on-skin wearable device.

FIG. 55 illustrates a perspective view of a socket.

FIG. 56 illustrates an exploded view of components including the socket shown in FIG. 55.

FIG. 57 illustrates an assembled view of the components shown in FIG. 56.

FIG. 58 illustrates a perspective view of an on-skin wearable housing including the components shown in FIG. 56.

FIG. 59 illustrates a perspective view of an analyte sensor system.

FIG. 60 illustrates a perspective view of an analyte sensor system.

FIG. 61 illustrates a perspective view of an analyte sensor system above a socket.

FIG. 62 illustrates a perspective view of a housing of an analyte sensor system.

FIG. 63 illustrates an exploded view of an analyte sensor module including a planar sensor.

FIG. 64 illustrates a top view of an elongate sensor.

FIG. 65 illustrates a perspective view of the elongate sensor shown in FIG. 64.

FIG. 66 illustrates a top view of an opposite side of a planar substrate of the elongate sensor shown in FIG. 64.

FIG. 67 illustrate a top perspective view of a sensor module.

FIG. 68 illustrates a side cross sectional view of the sensor module of FIG. 67 deployed within a socket.

FIG. 69 illustrates a top perspective view of a sensor module.

FIG. 70 illustrates a side cross sectional view of the sensor module of FIG. 69 deployed within a socket.

FIG. 71 illustrates a perspective view of a sensor module.

FIG. 72 illustrates a perspective view of the sensor module of FIG. 71 from an opposite side than shown in FIG. 71.

FIG. 73 illustrates a side view of the sensor module of FIG. 71.

FIG. 74 illustrates a perspective view of the frame of the sensor module of FIG. 71.

FIG. 75 illustrates a perspective view of the frame shown in FIG. 74 from an opposite side than shown in FIG. 74.

FIG. 76 illustrates a perspective view of the frame and electrical contacts of the sensor module of FIG. 71.

FIG. 77 illustrates a perspective view of the frame and electrical contacts shown in FIG. 76 from an opposite side than shown in FIG. 76.

FIG. 78 illustrates a cross sectional view of the sensor module of FIG. 71 along line IV-IV shown in FIG. 71.

FIG. 79 illustrates a cross sectional view of the sensor module of FIG. 71 along line V-V shown in FIG. 72.

FIG. 80 illustrates a cross sectional view of the sensor module of FIG. 71 along line VI-VI shown in FIG. 72.

FIG. 81 illustrates a perspective view of the sensor module of FIG. 71 being deployed to a socket.

FIG. 82 illustrates a perspective view of the sensor module of FIG. 71 deployed to a socket.

FIG. 83 illustrates a partial cross sectional view of the sensor module of FIG. 71 deployed to a socket.

FIG. 84 illustrates a perspective view of a sensor module.

FIG. 85 illustrates a perspective view of the sensor module of FIG. 84 from an opposite side than shown in FIG. 84.

FIG. 86 illustrates a perspective view of a sensor module.

FIG. 87 illustrates a perspective view of the sensor module of FIG. 86 from an opposite side than shown in FIG. 86.

FIG. 88 illustrates a side view of the sensor module of FIG. 86.

FIG. 89 illustrates a perspective view of an elongate sensor.

FIG. 90 illustrates a side view of the elongate sensor shown in FIG. 89.

FIG. 91 illustrates a perspective view of the frame of the sensor module of FIG. 86.

FIG. 92 illustrates a perspective view of the frame shown in FIG. 91 from an opposite side than shown in FIG. 91.

FIG. 93 illustrates a perspective view of electrical contacts molded to a planar substrate.

FIG. 94 illustrates a perspective view of the frame and electrical contacts of the sensor module of FIG. 86.

FIG. 95 illustrates a perspective view of the frame and electrical contacts shown in FIG. 94 from an opposite side than shown in FIG. 94.

FIG. 96 illustrates a cross sectional view of the sensor module of FIG. 86 along line VII-VII shown in FIG. 86.

FIG. 97 illustrates a cross sectional view of the sensor module of FIG. 86 along line VIII-VIII shown in FIG. 87.

FIG. 98 illustrates a cross sectional view of the sensor module of FIG. 86 along line IX-IX shown in FIG. 87.

FIG. 99 illustrates a perspective view of a sensor module.

FIG. 100 illustrates a perspective view of a sensor module.

FIG. 101 illustrates a perspective view of the sensor module of FIG. 86 being deployed to a socket.

FIG. 102 illustrates a perspective view of the sensor module of FIG. 86 deployed to a socket.

FIG. 103 illustrates a partial cross sectional view of the sensor module of FIG. 86 deployed to a socket.

FIG. 104 illustrates a perspective view of the sensor module of FIG. 86 deployed to a socket, at an opposite side of the wearable housing than shown in FIG. 102 and with an upper housing of the wearable housing shown in transparency.

FIG. 105 illustrates a perspective view of a sensor module.

FIG. 106 illustrates a perspective view of the sensor module of FIG. 105 from an opposite side than shown in FIG. 105.

FIG. 107 illustrates a side view of the sensor module of FIG. 105.

FIG. 108 illustrates a perspective view of an elongate sensor.

FIG. 109 illustrates a side view of the elongate sensor shown in FIG. 108 at an opposite side than shown in FIG. 108.

FIG. 110 illustrates a perspective view of the frame of the sensor module of FIG. 105.

FIG. 111 illustrates a perspective view of the frame shown in FIG. 110 from an opposite angle than shown in FIG. 110.

FIG. 112 illustrates a perspective view of the frame of the sensor module of FIG. 105 from an opposite side than shown in FIG. 110.

FIG. 113 illustrates a perspective view of electrical contacts adjacent to sensor electrical contacts of an elongate sensor.

FIG. 114 illustrates a perspective view of the elongate sensor and electrical contacts of FIG. 113 at an inverted position than shown in FIG. 113.

FIG. 115 illustrates a perspective view of the frame and electrical contacts of the sensor module of FIG. 105.

FIG. 116 illustrates a perspective view of the frame and electrical contacts shown in FIG. 115 from an opposite side than shown in FIG. 115.

FIG. 117 illustrates a cross sectional view of the sensor module of FIG. 105 along line X-X shown in FIG. 105.

FIG. 118 illustrates a cross sectional view of the sensor module of FIG. 105 along line XI-XI shown in FIG. 106.

FIG. 119 illustrates a cross sectional view of the sensor module of FIG. 105 along line XII-XII shown in FIG. 106.

FIG. 120 illustrates a perspective view of a sensor module.

FIG. 121 illustrates a perspective view of a sensor module.

### DETAILED DESCRIPTION

The following description and examples illustrate some example implementations of the disclosure in detail. Those of skill in the art will recognize that there are numerous variations and modifications of the disclosure that are encompassed by its scope. Accordingly, the description of a certain example implementation should not be deemed to limit the scope of the present disclosure.

In vivo analyte sensing technology may rely on in vivo sensors. In vivo sensors may include an elongate sensor or elongate analyte sensor wire having one or more electrodes such as a working electrode and a reference electrode.

For example, a platinum metal-clad, tantalum wire is sometimes used as a core bare sensing element with one or more reference or counter electrodes for an analyte sensor wire. This sensing element is coated in membranes to yield the final sensor wire. Other forms of sensors that may be utilized are disclosed in U.S. patent application Ser. No. 16/854,647, entitled “Preconnected Analyte Sensors,” and filed on April 21, 202, published U.S. Publication No. 2020/0330010 on Oct. 22, 2020, herein incorporated by reference in its entirety.

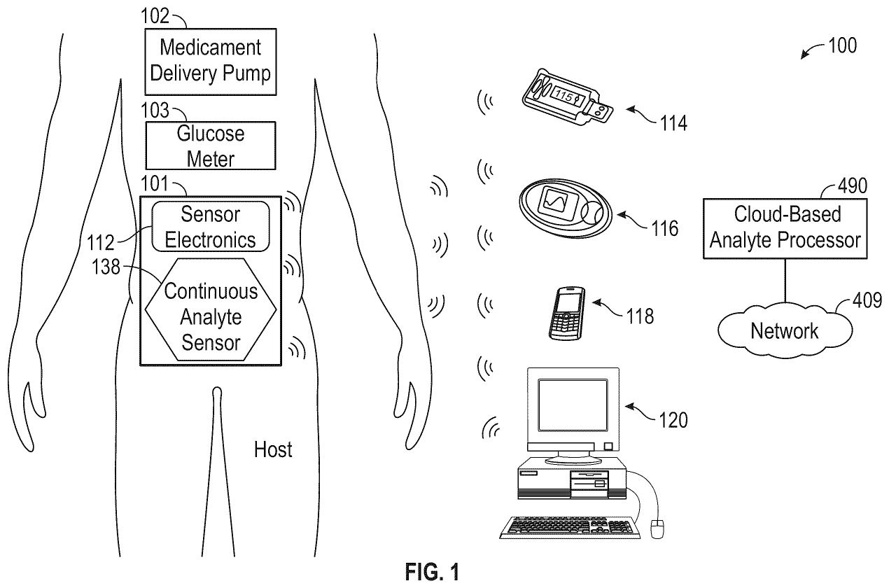

FIG. 1 depicts an example system 100, in accordance with some example implementations. The system 100 may include an on-skin wearable system 101 including sensor electronics 112 or a sensor electronics module, and an analyte sensor 138. The system 100 may include other medical devices and/or sensors, such as medicament delivery pump 102 and glucose meter 103. The analyte sensor 138 may be electrically connected to sensor electronics 112 in examples. The sensor electronics 112, medicament delivery pump 102, and/or glucose meter 103 may couple with one or more devices, such as display devices 114, 116, 118, and/or 120.

In some example implementations, the system 100 may include a cloud-based analyte processor 490 configured to analyze analyte data (and/or other patient-related data) provided via network 409 (e.g., via wired, wireless, or a combination thereof) from on-skin wearable system 101 and other devices, such as display devices 114, 116, 118, and/or 120 and the like, associated with the host (also referred to as a patient) and generate reports providing high-level information, such as statistics, regarding the measured analyte over a certain time frame. A full discussion of using a cloud-based analyte processing system may be found in U.S. patent application Ser. No. 13/788,375, entitled “Cloud-Based Processing of Analyte Data” and filed on Mar. 7, 2013, published as U.S. Patent Application Publication 2013/0325352, herein incorporated by reference in its entirety. In some implementations, one or more steps of the factory calibration algorithm can be performed in the cloud.

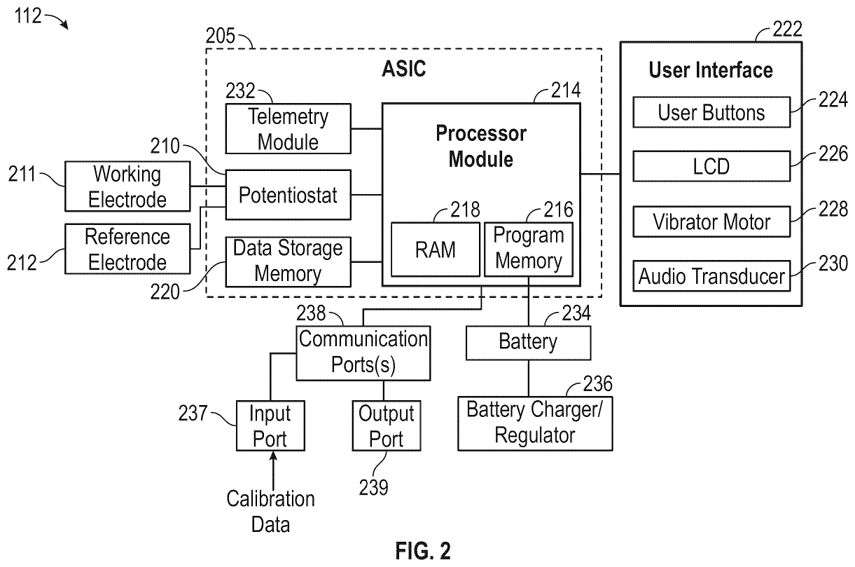

In some example implementations, electrical components in the form of sensor electronics 112 may include electronic circuitry associated with measuring and processing data generated by the analyte sensor 138. This generated analyte sensor data may also include algorithms, which can be used to process and calibrate the analyte sensor data, although these algorithms may be provided in other ways as well. The sensor electronics 112 may include hardware, firmware, software, or a combination thereof, to provide measurement of levels of the analyte via an analyte sensor, such as a glucose sensor. An example implementation of the sensor electronics 112 is described further below with respect to FIG. 2. In one implementation, factory calibration algorithms may be performed by the sensor electronics.

The sensor electronics 112 may, as noted, couple (e.g., wirelessly and the like) with one or more devices, such as display devices 114, 116, 118, and/or 120. The display devices 114, 116, 118, and/or 120 may be configured for presenting information (and/or alarming), such as sensor information transmitted by the sensor electronics 112 for display at the display devices 114, 116, 118, and/or 120. In one implementation, factory calibration algorithms may be performed at least in part by the display devices.

In some example implementations, the relatively small, key fob-like display device 114 may comprise a wrist watch, a belt, a necklace, a pendent, a piece of jewelry, an adhesive patch, a pager, a key fob, a plastic card (e.g., credit card), an identification (ID) card, and/or the like. This small display device 114 may include a relatively small display (e.g., smaller than the large display device 116) and may be configured to display certain types of displayable sensor information, such as a numerical value, and an arrow, or a color code.

In some example implementations, the relatively large, hand-held display device 116 may comprise a hand-held receiver device, a palm-top computer, and/or the like. This large display device may include a relatively larger display (e.g., larger than the small display device 114) and may be configured to display information, such as a graphical representation of the sensor data including current and historic sensor data output by sensor system 100.

In some example implementations, the analyte sensor 138 may comprise a transcutaneous analyte sensor. The transcutaneous analyte sensor may be configured to generate a signal indicative of an analyte concentration in a host. The analyte sensor 138 may comprise a glucose sensor configured to measure glucose in the blood or interstitial fluid using one or more measurement techniques, such as enzymatic, chemical, physical, electrochemical, spectrophotometric, polarimetric, calorimetric, iontophoretic, radiometric, immunochemical, and the like. The analyte sensor 138 may be configured for detecting glucose within the skin of a host. The analyte sensor 138 may be configured to detect glucose within extracellular fluid of a host. In implementations in which the analyte sensor 138 includes a glucose sensor, the glucose sensor may comprise any device capable of measuring the concentration of glucose and may use a variety of techniques to measure glucose including invasive, minimally invasive, and non-invasive sensing techniques (e.g., fluorescence monitoring), to provide data, such as a data stream, indicative of the concentration of glucose in a host. The data stream may be sensor data (raw and/or filtered), which may be converted into a calibrated data stream used to provide a value of glucose to a host, such as a user, a patient, or a caretaker (e.g., a parent, a relative, a guardian, a teacher, a doctor, a nurse, or any other individual that has an interest in the wellbeing of the host). Moreover, the analyte sensor 138 may be implanted as at least one of the following types of analyte sensors: an implantable glucose sensor, a transcutaneous glucose sensor, implanted in a host vessel or extracorporeally, a subcutaneous sensor, a refillable subcutaneous sensor, an intravascular sensor.

Although the disclosure herein refers to some implementations that include an analyte sensor 138 comprising a glucose sensor, the analyte sensor 138 may comprise other types of analyte sensors as well. Moreover, although some implementations refer to the glucose sensor as an implantable glucose sensor, other types of devices capable of detecting a concentration of glucose and providing an output signal representative of glucose concentration may be used as well. Furthermore, although the description herein refers to glucose as the analyte being measured, processed, and the like, other analytes may be used as well including, for example, ketone bodies (e.g., acetone, acetoacetic acid and beta hydroxybutyric acid, lactate, etc.), glucagon, acetyl-CoA, triglycerides, fatty acids, intermediaries in the citric acid cycle, choline, insulin, cortisol, testosterone, and the like.

FIG. 2 depicts an example of electronics 112 that may be used in sensor electronics 112 or may be implemented in a manufacturing station such as a testing station, a calibration station, a smart carrier, or other equipment used during manufacturing of on-skin wearable system 101, in accordance with some example implementations. The sensor electronics 112 may include electronics components that are configured to process sensor information, such as sensor data, and generate transformed sensor data and displayable sensor information, e.g., via a processor module. For example, the processor module 214 may transform sensor data into one or more of the following: filtered sensor data (e.g., one or more filtered analyte concentration values), raw sensor data, calibrated sensor data (e.g., one or more calibrated analyte concentration values), rate of change information, trend information, rate of acceleration/deceleration information, sensor diagnostic information, location information, alarm/alert information, calibration information such as may be determined by factory calibration algorithms, smoothing and/or filtering algorithms of sensor data, and/or the like.

In some implementations, the processor module 214 is configured to achieve a substantial portion, if not all, of the data processing, including data processing pertaining to factory calibration. Processor module 214 may be integral to sensor electronics 112 and/or may be located remotely, such as in one or more of devices 114, 116, 118, and/or 120 and/or cloud 490. For example, in some implementations, processor module 214 may be located at least partially within a cloud-based analyte processor 490 or elsewhere in network 409.

In some example implementations, the processor module 214 may be configured to calibrate the sensor data, and the data storage memory 220 may store the calibrated sensor data points as transformed sensor data. Moreover, the processor module 214 may be configured, in some example implementations, to wirelessly receive calibration information from a display device, such as devices 114, 116, 118, and/or 120, to enable calibration of the sensor data from sensor 138. Furthermore, the processor module 214 may be configured to perform additional algorithmic processing on the sensor data (e.g., calibrated and/or filtered data and/or other sensor information), and the data storage memory 220 may be configured to store the transformed sensor data and/or sensor diagnostic information associated with the algorithms. The processor module 214 may further be configured to store and use calibration information determined from a factory calibration, as described below.

In some example implementations, the sensor electronics 112 may comprise an application-specific integrated circuit (ASIC) 205 coupled to a user interface 222. The ASIC 205 may further include a potentiostat 210, a telemetry module 232 for transmitting data from the sensor electronics 112 to one or more devices, such as devices 114, 116, 118, and/or 120, and/or other components for signal processing and data storage (e.g., processor module 214 and data storage memory 220). Although FIG. 2 depicts ASIC 205, other types of circuitry may be used as well, including field programmable gate arrays (FPGA), one or more microprocessors configured to provide some (if not all of) the processing performed by the sensor electronics 112, analog circuitry, digital circuitry, or a combination thereof.

In the example depicted in FIG. 2, through a first input port for sensor data the potentiostat 210 is coupled to an analyte sensor 138, such as a glucose sensor to generate sensor data from the analyte. The potentiostat 210 may be coupled to a working electrode 211 and reference electrode 212 that form a part of sensor 138. The potentiostat may provide a voltage to one of the electrodes 211, 212 of analyte sensor 138 to bias the sensor for measurement of a value (e.g., a current) indicative of the analyte concentration in a host (also referred to as the analog portion of the sensor). The potentiostat 210 may have one or more connections to sensor 138 depending on the number of electrodes incorporated into the analyte sensor 138 (such as a counter electrode as a third electrode).

In some example implementations, the potentiostat 210 may include a resistor that translates a current value from sensor 138 into a voltage value, while in some example implementations, a current-to-frequency converter (not shown) may also be configured to integrate continuously a measured current value from sensor 138 using, for example, a charge-counting device. In some example implementations, an analog-to-digital converter (not shown) may digitize the analog signal from sensor 138 into so-called “counts” to allow processing by the processor module 214. The resulting counts may be directly related to the current measured by the potentiostat 210, which may be directly related to an analyte level, such as a glucose level, in the host.

The telemetry module 232 may be operably connected to processor module 214 and may provide the hardware, firmware, and/or software that enable wireless communication between the sensor electronics 112 and one or more other devices, such as display devices, processors, network access devices, and the like. A variety of wireless radio technologies that can be implemented in the telemetry module 232 include Bluetooth, Bluetooth Low-Energy, ANT, ANT+, ZigBee, IEEE 802.11, IEEE 802.16, cellular radio access technologies, radio frequency (RF), infrared (IR), paging network communication, magnetic induction, satellite data communication, spread spectrum communication, frequency hopping communication, near field communications, and/or the like. In some example implementations, the telemetry module 232 comprises a Bluetooth chip, although Bluetooth technology may also be implemented in a combination of the telemetry module 232 and the processor module 214. The telemetry module 232 may comprise a transmitter in implementations.

The processor module 214 may control the processing performed by the sensor electronics 112. For example, the processor module 214 may be configured to process data (e.g., counts), from the sensor, filter the data, calibrate the data, perform fail-safe checking, and/or the like.

Potentiostat 210 may measure the analyte (e.g., glucose and/or the like) at discrete time intervals or continuously, for example, using a current-to-voltage or current-to-frequency converter.

The processor module 214 may further include a data generator (not shown) configured to generate data packages for transmission to devices, such as the display devices 114, 116, 118, and/or 120. Furthermore, the processor module 214 may generate data packets for transmission to these outside sources via telemetry module 232. In some example implementations, the data packages may include an identifier code for the sensor and/or sensor electronics 112, raw data, filtered data, calibrated data, rate of change information, trend information, error detection or correction, and/or the like.

The processor module 214 may also include a program memory 216 and other memory 218. The processor module 214 may be coupled to a communications interface, such as a communication port 238, and a power source, such as a battery 234. Moreover, the battery 234 may be further coupled to a battery charger and/or regulator 236 to provide power to sensor electronics 112 and/or charge the battery 234.

The program memory 216 may be implemented as a semi-static memory for storing data, such as an identifier for a coupled sensor 138 (e.g., a sensor identifier (ID)) and for storing code (also referred to as program code) to configure the ASIC 205 to perform one or more of the operations/functions described herein. For example, the program code may configure processor module 214 to process data streams or counts, filter, perform the calibration methods described below, perform fail-safe checking, and the like.

The memory 218 may also be used to store information. For example, the processor module 214 including memory 218 may be used as the system's cache memory, where temporary storage is provided for recent sensor data received from the sensor. In some example implementations, the memory may comprise memory storage components, such as read-only memory (ROM), random-access memory (RAM), dynamic-RAM, static-RAM, non-static RAM, electrically erasable programmable read only memory (EEPROM), rewritable ROMs, flash memory, and the like.

The data storage memory 220 may be coupled to the processor module 214 and may be configured to store a variety of sensor information. In some example implementations, the data storage memory 220 stores one or more days of analyte sensor data. The stored sensor information may include one or more of the following: a time stamp, raw sensor data (one or more raw analyte concentration values), calibrated data, filtered data, transformed sensor data, and/or any other displayable sensor information, calibration information (e.g., reference BG values and/or prior calibration information such as from factory calibration), sensor diagnostic information, and the like.

The user interface 222 may include a variety of interfaces, such as one or more buttons 224, a liquid crystal display (LCD) 226, a vibrator 228, an audio transducer (e.g., speaker) 230, a backlight (not shown), and/or the like. The components that comprise the user interface 222 may provide controls to interact with the user (e.g., the host).

The power source or battery 234 may be operatively connected to the processor module 214 (and possibly other components of the sensor electronics 112) and provide the necessary power for the sensor electronics 112. In other implementations, the receiver can be transcutaneously powered via an inductive coupling, for example.

A battery charger and/or regulator 236 may be configured to receive energy from an internal and/or external charger. In some example implementations, the battery 234 (or batteries) is configured to be charged via an inductive and/or wireless charging pad, although any other charging and/or power mechanism may be used as well.

One or more communication ports 238, also referred to as external connector(s), may be provided to allow communication with other devices, for example a PC communication (com) port can be provided to enable communication with systems that are separate from, or integral with, the sensor electronics 112. The communication port, for example, may comprise a serial (e.g., universal serial bus or “USB”) communication port, and allow for communicating with another computer system (e.g., PC, personal digital assistant or “PDA,” server, or the like). In some example implementations, factory information may be sent to the algorithm from the sensor or from a cloud data source.

The one or more communication ports 238 may further include an input port 237 in which calibration data may be received, and an output port 239 which may be employed to transmit calibrated data, or data to be calibrated, to a receiver or mobile device. FIG. 2 illustrates these aspects schematically. It will be understood that the ports may be separated physically, but in alternative implementations a single communication port may provide the functions of both the second input port and the output port.

In some on-skin wearable systems, an on-skin portion of the sensor electronics may be simplified to minimize complexity and/or size of on-skin electronics, for example, providing only raw, calibrated, and/or filtered data to a display device configured to run calibration and other algorithms required for displaying the sensor data. However, the sensor electronics 112 (e.g., via processor module 214) may be implemented to execute prospective algorithms used to generate transformed sensor data and/or displayable sensor information, including, for example, algorithms that: evaluate a clinical acceptability of reference and/or sensor data, evaluate calibration data for best calibration based on inclusion criteria, evaluate a quality of the calibration, compare estimated analyte values with time corresponding measured analyte values, analyze a variation of estimated analyte values, evaluate a stability of the sensor and/or sensor data, detect signal artifacts (noise), replace signal artifacts, determine a rate of change and/or trend of the sensor data, perform dynamic and intelligent analyte value estimation, perform diagnostics on the sensor and/or sensor data, set modes of operation, evaluate the data for aberrancies, and/or the like. The sensor electronics 112 may comprise a transmitter in implementations.

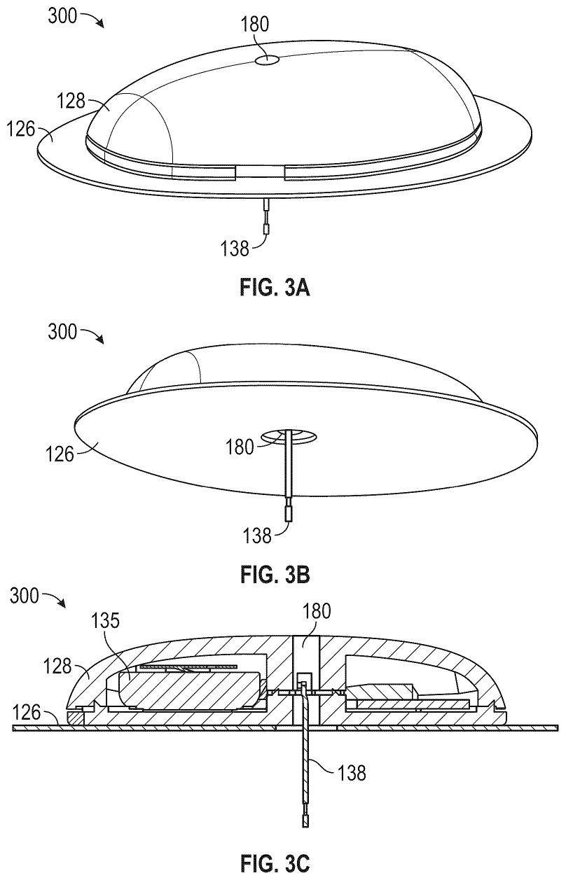

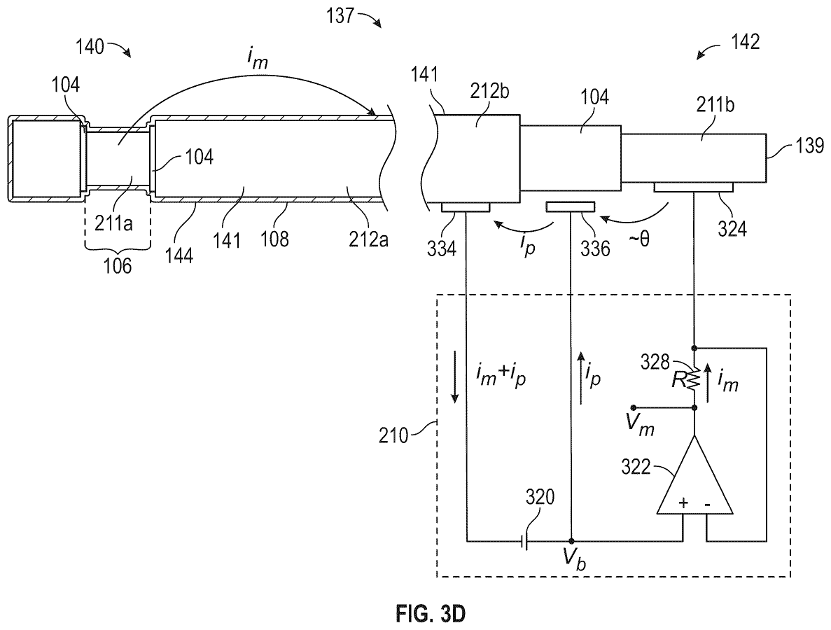

FIGS. 3A, 3B, and 3C illustrate an exemplary implementation of on-skin wearable system 101 implemented as a wearable device such as an on-skin wearable medical device or sensor assembly 300. As shown in FIG. 3A, on-skin sensor assembly comprises a body in the form of a wearable housing 128. The wearable housing 128 is for wear on skin of a host. A patch 126 can couple the wearable housing 128 to the skin of the host. The adhesive of the patch 126 can be a pressure sensitive adhesive (e.g. acrylic, rubber based, or other suitable type) bonded to a carrier substrate (e.g., spun lace polyester, polyurethane film, or other suitable type) for skin attachment. The wearable housing 128 may include a through-hole 180 that cooperates with an applicator such as a sensor inserter device (e.g., a sensor insertion needle, not shown) that is used for implanting sensor 138 under the skin of a host.

The wearable sensor assembly 300 can include electrical components in the form of sensor electronics 112 (e.g., as at least a portion of electronics module 135) operable to measure and/or analyze glucose indicators sensed by glucose sensor 138. Sensor electronics 112 within sensor assembly 300 can transmit information (e.g., measurements, analyte data, and glucose data) to a remotely located device (e.g., 114, 116, 118, 120 shown in FIG. 1). As shown in FIG. 3C, in this implementation sensor 138 extends from its distal end up into through-hole 180 and is routed to electrical components in the form of an electronics module 135 inside the enclosure or wearable housing 128. The working electrode 211 and reference electrode 212 are electrically connected to circuitry in the electronics module 135 which includes the potentiostat.

FIG. 3D illustrates an exemplary embodiment of an analyte sensor comprising an elongate sensor 137 or elongate analyte sensor wire. The elongate sensor 137 includes an elongated body portion. The elongated body portion may be long and thin, yet flexible and strong. For example, in some embodiments, the smallest dimension of the elongated conductive body is less than about 0.1 inches, 0.075 inches, 0.05 inches, 0.025 inches, 0.01 inches, 0.004 inches, or 0.002 inches. While the elongated conductive body is illustrated herein as having a circular cross-section, in other embodiments the cross-section of the elongated conductive body can be ovoid, rectangular, triangular, or polyhedral, star-shaped, C-shaped, T-shaped, X-shaped, Y-shaped, irregular, or the like.

In the implementation of FIG. 3D, the elongate sensor 137 comprises a wire core 139. At a distal end portion 140 or in vivo portion of elongate sensor 137, the wire core 139 forms an electrode 211a. At a proximal end portion 142 or ex vivo portion of elongate sensor 137, the wire core 139 forms an electrical contact 211b. The electrode 211a and the electrical contact 211b are in electrical communication over the length of the wire core 139 as it extends along the elongated body portion of elongate sensor 137. The wire core can be made from a single material such as platinum or tantalum, or may be formed as multiple layers, such as a conducting or non-conducting material with an outer coating of a different conducting material.

In examples, the distal end portion 140 of the elongate sensor 137 may be configured to be inserted into skin of a host for detecting an analyte within the skin of the host. The proximal end portion 142 of the elongate sensor 137 may be configured to be positioned exterior of the skin of the host. The proximal end portion 142 may include one or more sensor electrical contacts or electrical contacts 211b, 212b of the elongate sensor 137.

A layer 104 may surround a least a portion of the wire core 139. The layer 104 may be formed of an insulating material, such as polyimide, polyurethane, parylene, or any other known insulating materials. For example, in one embodiment the layer 104 is disposed on the wire core 139 and is configured such that the electrode 211a is exposed via window 106.

In some embodiments, elongate sensor 137 further comprises a layer 141 surrounding the insulating layer 104 like a sleeve that comprises a conductive material. At the distal end portion 140 or in vivo portion of the elongate sensor 137, the sleeve layer 141 forms an electrode 212a. At a proximal end portion 142 or ex vivo portion of elongate sensor 137, the sleeve layer 141 forms a contact 212b. The electrode 212a and the contact 212b are in electrical communication over the length of the sleeve layer 141 as it extends along the elongated body portion of elongate sensor 137. This sleeve layer 141 may be formed of a silver-containing material that is applied onto the insulating layer 104. The silver-containing material may include any of a variety of materials and be in various forms, such as, Ag/AgCl-polymer pastes, paints, polymer-based conducting mixture, and/or inks that are commercially available, for example. This layer 141 can be processed using a pasting/dipping/coating step, for example, using a die-metered dip coating process. In one exemplary embodiment, an Ag/AgCl polymer paste is applied to an elongated body by dip-coating the body (e.g., using a meniscus coating technique) and then drawing the body through a die to meter the coating to a precise thickness. In some embodiments, multiple coating steps are used to build up the coating to a predetermined thickness.

Elongate sensor 137 shown in FIG. 3D also includes a membrane 108 covering at least a portion of the distal end portion 140 or in vivo portion of elongate sensor 137. This membrane 108 is typically formed of multiple layers, which may include one or more of an interference domain, an enzyme domain, a diffusion resistance domain, and a bioprotective domain. This membrane is important to support the electrochemical processes that allow analyte detection and it is generally manufactured with great care by dip-coating, spraying, or other manufacturing steps. It is preferable for the distal end portion 140 or in vivo portion of elongate sensor 137 to be subject to as little handling as possible or practical from the time that the membrane 108 is formed to the time that the distal end portion 140 or in vivo portion of elongate sensor 137 is implanted into a subject. In some embodiments, electrode 211a forms a working electrode of an electrochemical measuring system, and electrode 212a forms a reference electrode for that system. In use, both electrodes may be implanted into a host for analyte monitoring.

The elongate sensor 137 has an outer surface 144, which may comprise the outer surface of the membrane 108 or another outer surface of the elongate sensor 137. The outer surface 144 may comprise a rough outer surface or a smooth outer surface, or combinations thereof in examples.

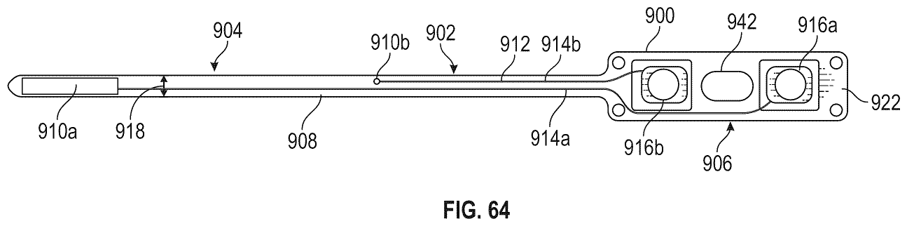

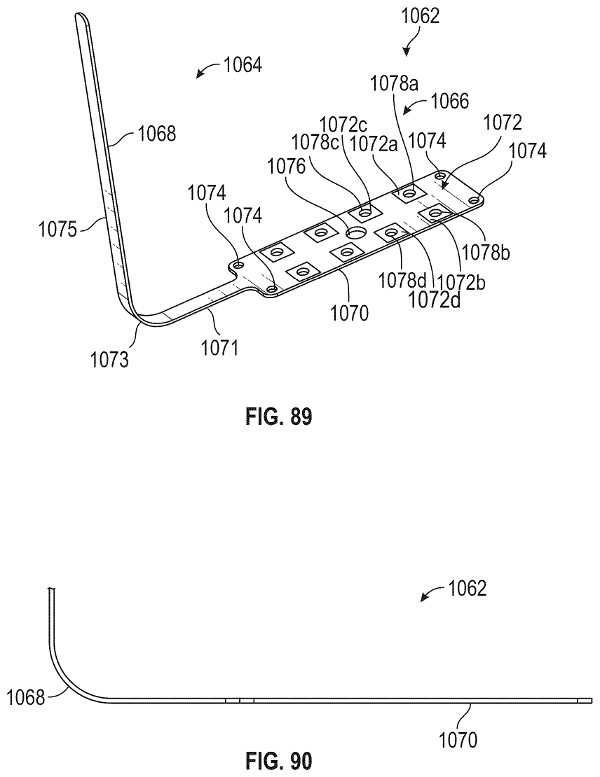

Although the above description is applicable specifically to a coaxial sensor wire having a coaxial wire type structure, the embodiments herein are also applicable to other physical configurations of electrodes. For example, the elongate sensor may include two electrodes 211a and 212a that could be affixed to a distal end portion or in vivo portion of an elongated flexible strip of a planar substrate such as a thin, flat, polymer flex circuit. The two contacts 211b and 212b could be affixed to the proximal end portion or ex vivo portion of this flexible planar substrate. An exemplary configuration is represented in FIGS. 64 and 89 (and FIG. 108). Electrodes 211a, 212a could be electrically connected to their respective contacts 211b, 212b via circuit traces on the planar substrate. In this case, the electrodes 211a and 212a and the contacts 211b and 212b may be adjacent to one another on a flat surface rather than being coaxial as shown in FIG. 3D. Examples as disclosed herein may be applied to coaxial sensor wires or flat or planar sensors.

Also shown in FIG. 3D is an illustration of the contact 211b and the contact 212b electrically coupled to a simple current-to-voltage converter based potentiostat 210. The potentiostat includes a battery 320 that has an output coupled to an input of an operational amplifier 322. The output of the operational amplifier 322 is coupled to a contact 324 that is electrically coupled to the working electrode contact 211b through a resistor 328. The amplifier 322 will bias the contact 324 to the battery voltage Vb, and will drive the current Iₘ required to maintain that bias. This current will flow from the working electrode 211a through the interstitial fluid surrounding elongate sensor 137 and to the reference electrode 212a. The reference electrode contact 212b is electrically coupled to another contact 334 which is connected to the other side of the battery 320. For this circuit, the current iₘ is equal to (V_{b}−Vₘ)/R, where Vₘ is the voltage measured at the output of the amplifier 322. The magnitude of this current for a given bias on the working electrode 211a is a measure of analyte concentration in the vicinity of the window 106.

The contacts 324 and 334 may be conductive pads/traces on a circuit board. There may be some level of parasitic leakage current iₚ over the surface of this board during the test. If possible, this leakage current should not form part of the measurement of current due to analyte. To reduce the effect this leakage current has on the measured current, an optional additional pad/trace 336 may be provided between the biased contact 324 and the return contact 334 that is connected directly to the battery output. This optional additional pad/trace may be referred to as a “guard trace.” Because they are held at the same potential, there will be essentially no leakage current from the biased contact 324 and the guard trace 336. Furthermore, leakage current from the guard trace 336 to the return contact 334 does not pass through the amplifier output resistor 328, and therefore is not included in the measurement. Additional aspects and implementations of a guard trace may be found in paragraphs and of U.S. Patent Publication 2017/0281092, which are incorporated herein by reference.

It may be difficult to handle, connect, and/or incorporate an elongate sensor during manufacturing, testing, assembly, shipping, or other processes applied to the elongate sensor. The relatively small size, fragility, sensitivity, and/or flexibility of an elongate sensor may produce such difficulties. Systems, methods, and apparatuses may be provided to address such issues.

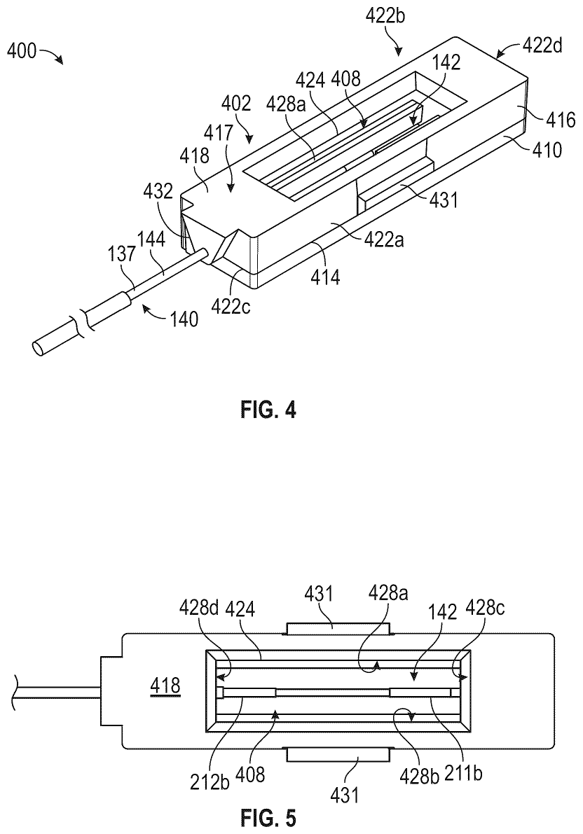

FIG. 4 illustrates a perspective view of an analyte sensor system 400 that may be utilized in examples herein. The analyte sensor system 400 may also be referred to as an analyte sensor module or sensor module, and may include an elongate sensor (for example, the elongate sensor 137 disclosed in regard to FIG. 3D), and may include a body 402 which may be molded upon, e.g. insert molded or overmolded upon, at least a portion of the elongate sensor 137. The body 402 may have exemplary dimensions of about 1 millimeter in height, 2 millimeters in width, and 8 millimeters in length, although other dimensions may be utilized as desired. A range of between 0.5 millimeters and 2 millimeters in height, between 1 millimeter and 3 or 5 millimeters in width, and between 5 millimeters and 10 millimeters in length may be utilized, although other dimensions may be utilized as desired.

An injection molding process may form the body 402 upon at least the portion of the elongate sensor 137 within a mold. For example, the proximal end portion 142 of the elongate sensor 137 may be inserted into a mold. The mold may be shaped and dimensioned to produce the desired corresponding shape and dimensions of the body 402 upon the elongate sensor 137. The material forming the body 402 may be injected into the mold to thereby form the body 402 and may be cooled rapidly. The body 402 is physically bonded to at least a portion of the elongate sensor 137.

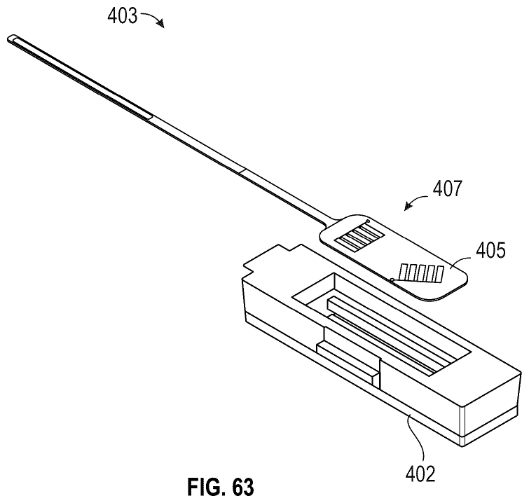

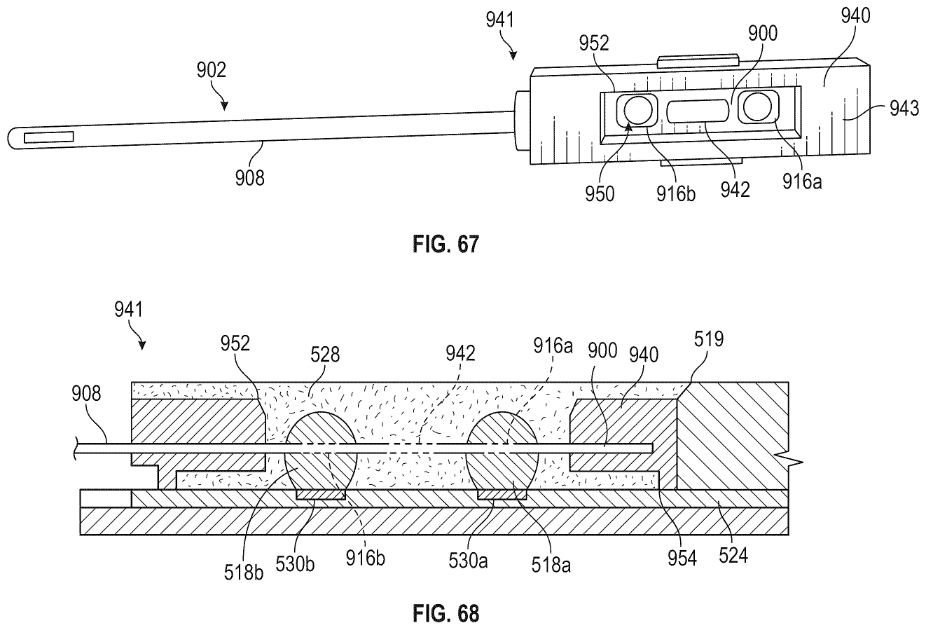

The elongate sensor 137 may have a variety of forms, and may be manufactured utilizing methods disclosed herein. In examples, a co-axial sensor wire may be utilized that may be manufactured by annealing a wire (e.g., tantalum wire) from a relatively large diameter (e.g., 0.020 inches) down to a smaller diameter (e.g., 0.006 inches) in multiple steps. The wire may be coated with one or more layers (e.g., a polyurethane layer) and then additional layers (e.g., a silver/silver chloride layer). Other forms of sensors may be utilized. A flat (e.g., planar) sensor may be utilized in examples. FIG. 63, for example, illustrates an exploded view of an analyte sensor module comprising a planar or flat sensor 403 and an overmolded body 402, in accordance with one embodiment. FIG. 67 illustrates a body having been molded onto a planar or flat sensor.

In examples, a shot of the molten injection molded material may be injected at high speed under pressure. The injection molded material may cool rapidly (e.g., at a fraction of a second). The material of the body 402 may comprise a thermoplastic resin (e.g., polypropylene, or other forms of plastics or other materials). The material of the body 402 may be non-conductive in examples. The material of the body 402 may be rigid after cooling. A rigid thermoplastic material may be utilized. The shot may physically or mechanically bond with the silver/silver chloride or other material comprising an outer surface of the elongate sensor, thereby sealing and mechanically fixing the elongate sensor in place.

The distal end portion 140 of the elongate sensor 137 may remain exterior to the mold during the molding process. For example, the distal end portion 140 of the elongate sensor 137 may be held or otherwise retained outside of the mold during the overmolding process. The mold may comprise a micro-mold configured to allow for only a portion or portions of the elongate sensor 137 to have material molded thereon. The mold may comprise a tight tolerance injection mold cavity. Other forms of molding techniques may be utilized in examples.

The body 402 as shown in FIG. 4 may comprise a single body or unitary body formed from a single shot of an injection molding process upon the proximal end portion 142 of the elongate sensor 137. In examples, multiple shots or molding processes may be utilized to form any portion of the body 402.

The body 402 may be in direct contact with the outer surface 144 of the elongate sensor 137 upon formation. As such, the body 402 may form with a contour to the shape of a portion of the elongate sensor 137 that the body 402 is overmolded upon. For example, referring to the cross sectional view of FIG. 7, the body 402 is formed in direct contact with portions 404, 406 of the elongate sensor 137 and tightly fits to the outer surface 144 of the elongate sensor 137 at these portions 404, 406. The tight fit of the body 402 to the portions 404, 406 of the elongate sensor 137 may form a bond with or seal upon the outer surface 144 to prevent passage of substances therebetween (e.g., liquids, moisture, and/or air or other gases). In examples, other forms of molding may be utilized. The seals may prevent passage of such substances to an interior chamber 408 of the body 402.

Referring back to FIG. 4, the body 402 may comprise a housing 410 or elongate sensor housing that houses the proximal end portion 142 of the elongate sensor 137. The housing 410 is disposed at the proximal end portion 142 of the elongate sensor 137. The housing 410 may comprise a frame for supporting components of the analyte sensor system 400 in examples.

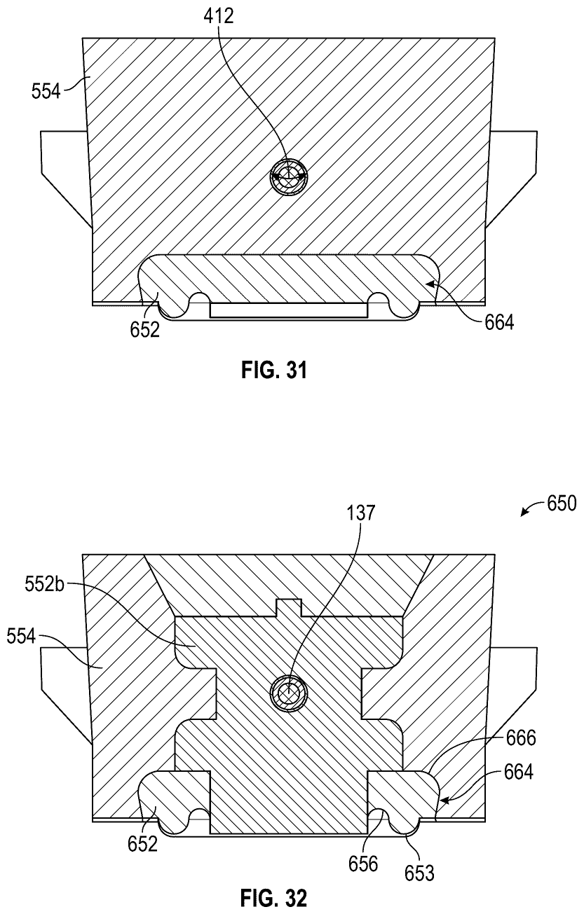

The housing 410 may be dimensioned larger than the proximal end portion 142 of the elongate sensor 137. The housing 410 may protrude radially outward from the outer surface 144 of the elongate sensor 137. The housing 410 may have a larger diameter than the diameter 412 (marked in FIG. 31) of the elongate sensor 137. The housing 410 may have a larger cross-sectional dimension than a cross sectional dimension of the proximal end portion 142 of the elongate sensor 137. As such, the housing 410 and/or the surface area 417 may be adapted for handling, whether manually or mechanically, with greater ease than the proximal end portion 142 of the elongate sensor 137 itself. The increased size of the housing 410 may provide for enhanced ease of handling. The housing 410 may further shield and protect the proximal end portion 142 of the elongate sensor 137 from damage, from, e.g., moisture or direct contact. The housing 410 may comprise a sensor carrier that provides for improved handling of the elongate sensor 137.

The housing 410 may extend along a length of the proximal end portion 142 of the elongate sensor 137. The housing 410 may comprise an elongate body having an elongate shape as shown in FIG. 4 for example, with a distal end portion 414 and a proximal end portion 416. The housing 410 may include one or more outer surfaces, which may be adapted for handling and/or coupling with corresponding structure in an on-skin device. The outer surfaces may comprise handling surfaces. Other handling surfaces may be provided in examples.

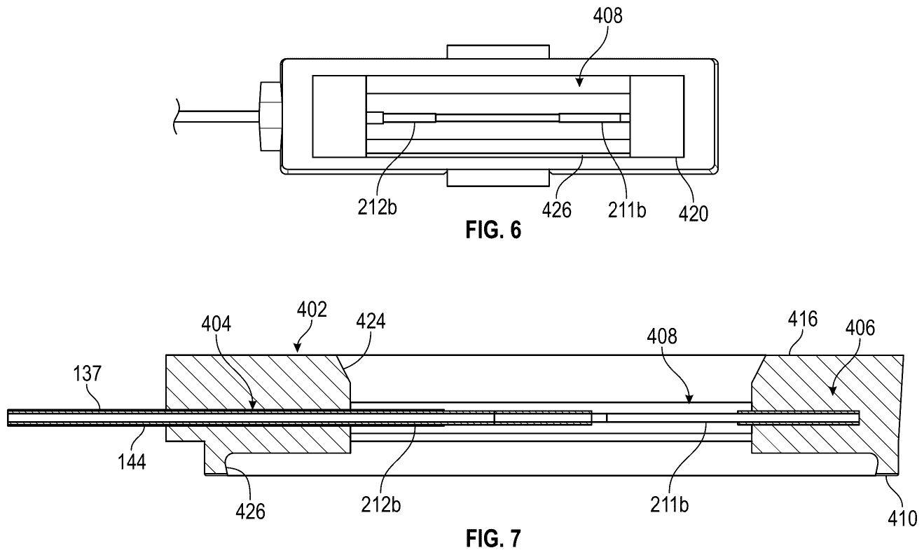

The outer surfaces may include an upper outer surface 418, a lower outer surface 420 (marked in FIG. 6) facing opposite the upper outer surface 418 and one or more outer side surfaces 422a-d extending between the upper outer surface 418 and the lower outer surface 420. The one or more outer side surfaces 422a-d may include a first outer side surface 422a and a second outer side surface 422b facing opposite the first outer side surface 422a. The first and second outer side surfaces 422a, b may extend along the length of the proximal end portion 142 of the elongate sensor 137. The one or more outer side surfaces 422a-d may include a distal outer end surface 422c and a proximal outer end surface 422d facing opposite the distal outer end surface 422c. The upper outer surface 418 and the lower outer surface 420 may each extend along the length of the proximal end portion 142 of the elongate sensor 137. The housing 410 may have a substantially rectangular outer profile as shown in FIG. 4 for example, or may have another shape in examples as desired (e.g., cuboid, or substantially cuboid). The proximal end portion 142 of the elongate sensor 137 may pass through a central interior portion of the housing 410 (e.g., within the volume defined by the housing 410).

The outer surfaces 418, 420, 422a-d may be flat surfaces, which may aid in mechanical handling (e.g., grip via graspers or vacuum grasping) of the housing 410. In examples, other shapes of surfaces (e.g., curved or undulating, or textured) may be utilized. Although a rectangular profile is shown in FIG. 4, other profiles (e.g., spherical, triangular, pentagonal, hexagonal, among others) may be utilized in examples.

The housing 410 may be formed upon the proximal end portion 142 of the elongate sensor 137 such that the elongate sensor 137 extends through the distal outer end surface 422c of the housing 410 and passes through the interior of the housing 410 towards the proximal outer end surface 422d of the housing 410. The elongate sensor 137 may pass through a central portion of the distal outer end surface 422c, or another position (e.g., at a side) of the housing 410. The housing 410 may entirely surround an outer perimeter or circumference of the outer surface 144 of the elongate sensor 137 along one or more portions of the elongate sensor 137.

In examples, the housing 410 may define an interior chamber 408 for receiving at least a portion of the elongate sensor 137. The elongate sensor 137 may extend through the interior chamber 408. The electrical contacts 211b, 212b of the elongate sensor 137 may be positioned within the interior chamber 408. The housing 410 may include one or more openings 424, 426 of the interior chamber 408. The one or more openings 424, 426 may allow for access to the elongate sensor 137 within the interior chamber 408, and may allow for access to the electrical contacts 211b, 212b of the elongate sensor 137 in examples. The one or more openings 424, 426 may allow for electrical connection of the electrical contacts 211b, 212b of the elongate sensor 137 with electrical contacts of a sensor electronics module.

The opening 424 may be positioned in the upper outer surface 418 of the housing 410 and the opening 426 (marked in FIG. 6) may be positioned in the lower outer surface 420 of the housing 410. The interior chamber 408 may extend between the openings 424, 426 in examples, although a single opening may be utilized in some examples.

The interior chamber 408 may have an oblong shape and may be bound by one or more surfaces that may include a first interior side surface 428a, a second interior side surface 428b, a proximal interior end surface 428c, and a distal interior end surface 428d. The first interior side surface 428a may face opposite the second interior side surface 428b. The interior side surfaces 428a, b may each extend along the length of the proximal end portion 142 of the elongate sensor 137. The elongate sensor 137 may pass through the distal interior end surface 428d to the interior chamber 408 and in examples may pass through the proximal interior end surface 428c. Referring to FIG. 7, a tip portion 406 of the elongate sensor 137 may be encapsulated within the proximal end portion 416 of the housing 410 in examples. The tip portion 406 may be positioned within the interior chamber 408 in examples.

The proximal end portion 142 of the elongate sensor 137 may pass through the interior chamber 408 along the length of the interior chamber 408 and may be accessible through one or more of the openings 424, 426. The electrical contacts 211b, 212b may be accessible for an electrical connection.

FIG. 7 illustrates a cross sectional view of the elongate sensor 137 and housing 410 illustrating the elongate sensor 137 passing through the interior chamber 408.

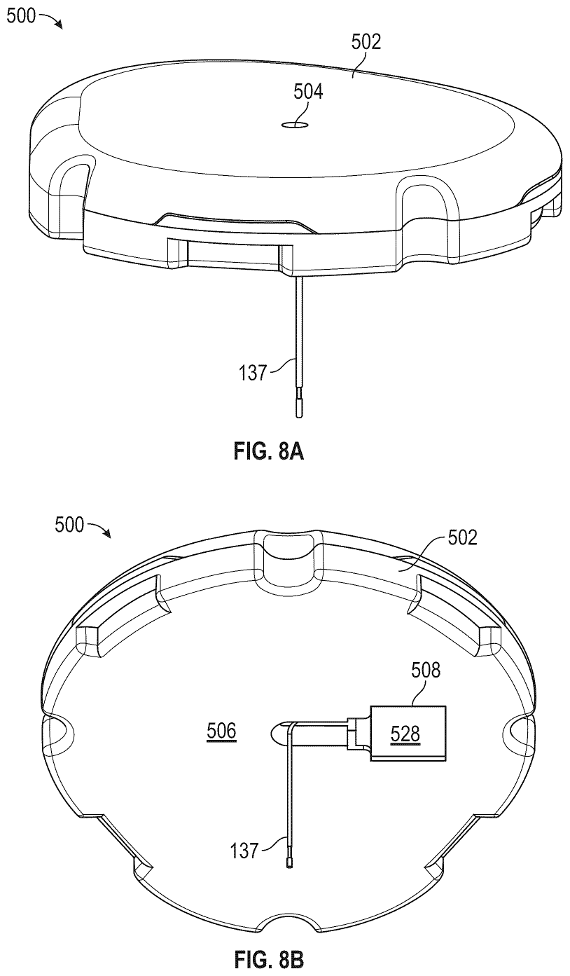

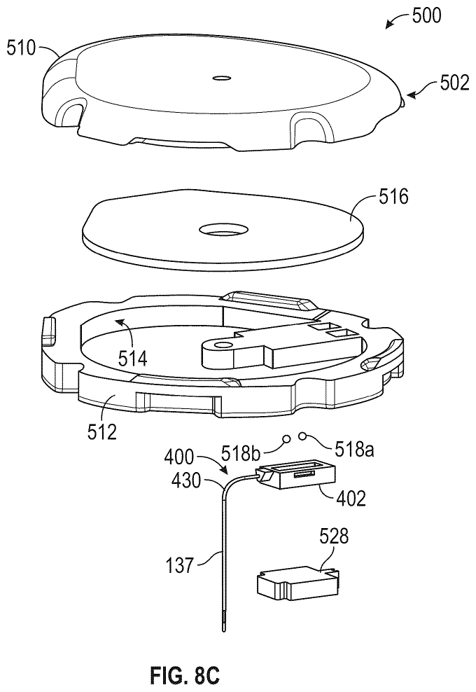

Referring to FIG. 4, the distal end portion 140 of the elongate sensor 137 protrudes from the body 402. The distal end portion 140 of the elongate sensor 137 accordingly is uncovered by the body 402 and the outer surface 144 of the distal end portion 140 of the elongate sensor 137 may remain exposed. In examples, the elongate sensor 137 may be bent or may include a bend 430 (marked in FIG. 8C) distal of the body 402 to fit desired geometry of the environment that is external of the analyte sensor system 400 and/or to allow the distal end portion 140 of the elongate sensor 137 to be inserted into the skin of a host. For example, a bend in an upward, downward, or side directions may be provided. The elongate sensor 137 may be a bent elongate sensor.

The body 402 may form at least a portion of a sensor module for the elongate sensor 137. The body 402 may allow for mechanical and/or electrical coupling of the elongate sensor 137 to one or more other apparatuses or systems (such as recesses or receptacles formed in a wearable device or sockets configured for coupling with the body 402). In examples, the one or more other apparatuses or systems may be for receiving a signal from the elongate sensor 137, such as an electrical signal. The electrical signal may indicate an analyte detected by the elongate sensor 137, such as an analyte concentration. The one or more other apparatuses or systems may electrically connect with one or more of the electrical contacts 211b, 212b of the elongate sensor 137. The body 402 may aid in the mechanical and/or electrical connection to external contacts that may electrically connect with one or more of the electrical contacts 211b, 212b of the elongate sensor 137. The electrical connection may be with electronics (e.g., sensor electronics or a sensor electronics module) for receiving an electrical signal from the elongate sensor 137. The sensor electronics may be the electronics of an on-skin wearable system, or may be the electronics of a sensor testing apparatus, among other forms of electronics.

The shape of the body 402 may provide for handling, releasable coupling to a fixture for coating of the elongate sensor, calibration, or testing, or mating to various other geometries as desired. The shape of the body 402 may provide for precise x-y-z positioning and anchoring of the elongate sensor relative to the body 402 and any electrical contacts.

In examples, the body 402 may include one or more mating features 431, 432 for mating the body 402 to a receiving portion of an external structure such as a socket. The mating features 431, 432 may be for mating with a corresponding structure of a wearable device. The mating features 431, for example, may comprise ridges or crush ribs that may protrude from an outer surface of the housing 410. Other forms of mating features (e.g., recesses or keyed features) may be utilized in examples. A mating feature 432 may be utilized to rotationally align the body 402 relative to a receiving portion or socket. The mating feature 432 may be positioned on the distal outer end surface 422c of the housing 410 or may have another position in examples. The mating feature 432 may comprise a wedge, angled, or curved surface, such as of a protrusion or recess, that may allow for alignment with a corresponding feature of a socket. Other forms and locations of mating features may be utilized in examples. In examples, the mating feature 432 may be utilized to form a seal against moisture traveling along the elongate sensor 137 and into the body 402.

FIGS. 8A-11 illustrate an exemplary connection of the analyte sensor system 400 to a portion of an on-skin wearable medical device 500. Referring to FIG. 8A, the on-skin wearable medical device 500 may include a wearable housing 502 for wear on the skin of a host. Features of other forms of on-skin wearable medical devices disclosed herein may be utilized as desired. The wearable housing 502, for example, may include a through hole 504 for an insertion element (e.g., a needle) to pass through for insertion of the elongate sensor 137.

Referring to FIG. 8B, a bottom perspective view of the on-skin wearable medical device 500 is shown. A bottom outer surface 506 of the wearable housing 502 may include a receiver portion in the form of a recess 508, which may comprise a socket or receiver for receiving the analyte sensor system 400. The analyte sensor system 400 and particularly the body 402 may be inserted into the recess 508. The socket or receiver may be coupled to or formed in or on the wearable housing 502 and may be configured to receive the body 402 of the analyte sensor system 400.

FIG. 8C illustrates an exploded view of the on-skin wearable medical device 500. The wearable housing 502 may include an upper or top housing 510 and a lower or bottom housing 512. An interior chamber 514 may be positioned between the upper housing 510 and the lower housing 512 that may receive electrical components of the on-skin wearable medical device 500 such as a circuit board substrate 516. The circuit board substrate 516 may be positioned within the interior chamber 514 and may include the sensor electronics 112 or a sensor electronics module for electrical connection with the elongate sensor 137. The sensor electronics 112 may be configured to receive an electrical signal from the elongate sensor 137.

The recess 508 may be formed in a wall of the wearable housing 502, and particularly in a wall of the lower or bottom housing 512. Other locations (e.g., in a wall of the upper or top housing 510) may be utilized.

In examples, one or more electrical contacts 518a, b may be utilized that may electrically connect with the electrical contacts 211b, 212b of the elongate sensor 137. The electrical contacts 518a, b may have a variety of forms in examples. As shown in FIG. 8C and in FIG. 10, the electrical contacts 518a, b may comprise a cured liquid or cured conductive liquid (e.g., a cured adhesive, gel, epoxy, etc.) that may be conductive and may allow for electrical connection with the electrical contacts 211b, 212b of the elongate sensor 137. The conductive liquid solidifies to form a solidified conductive liquid. In examples, other forms of electrical contacts (e.g., pads or pucks, protrusions such as pins, electrically conductive film, magnetic materials, magnetic conductive adhesive, uv curable materials such as uv curable die attach, among others) may be utilized as desired. The form of electrical contacts 518a, b is not limited to wet processes.

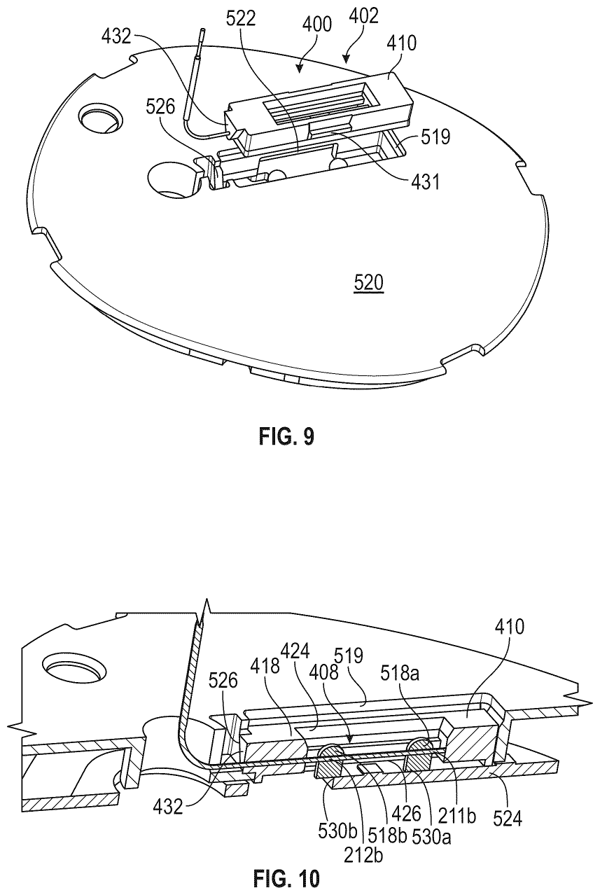

FIG. 9 illustrates the analyte sensor system 400 approaching a socket 519 of a wearable housing, which may be configured similarly as the wearable housing 502 shown in FIG. 8B. A bottom outer surface 520 of the wearable housing is shown, which may be configured similarly as the bottom outer surface 506 shown in FIG. 8B. The socket 519 may comprise a recess in the bottom outer surface 520. The socket 519 may include one or more mating features 522 for mating with the body 402. The mating features 522 may be configured to mate with the mating features 431 of the housing 410. For example, the mating features 522 may comprise recesses that may receive the ridges of the mating features 431. The mating features 431, 522 may aid to secure the housing 410 within the socket 519.

The socket 519 may be dimensioned and shaped to receive the housing 410. The housing 410 may insert into the socket 519 in a direction transverse to the length of the proximal end portion 142 of the elongate sensor 137. The direction may be perpendicular to the length of the proximal end portion 142 of the elongate sensor 137 and perpendicular to the length of the housing 410. In examples, other directions of insertion (e.g., longitudinal) may be utilized as desired.

The socket 519 may be positioned upon a circuit board substrate 524 (which may be configured similarly as the circuit board substrate 516). The housing 410 may insert into the socket 519 in a direction towards the circuit board substrate 524. A resulting cross sectional view of an inserted configuration is shown in FIG. 10. The one or more mating features 522 shown in FIG. 9 may mate with the mating features 431 of the housing 410. A press fit or friction fit within the socket 519 may be provided.

In examples, the socket 519 or receiver may include a mating feature 526 that may have a complementary shape to the angled mating feature 432. The mating features 526, 432 may serve to rotationally orient the housing 410 within the socket 519. The contact between the mating features 526, 432 may further form a dam for retaining encapsulant material within the socket 519.

The electrical contacts 211b, 212b of the elongate sensor 137 may be accessible for an electrical connection through the opening 426. The electrical contacts 518a, b, for example, may pass through the opening 426 for physical contact and electrical connection with the respective electrical contacts 211b, 212b of the elongate sensor 137. The electrical contacts 518a, b may be positioned upon respective electrical contacts 530a, b of the circuit board substrate 524. The electrical contacts 518a, b may electrically connect the electrical contacts 211b, 212b of the elongate sensor 137 with the respective electrical contacts 530a, b of the circuit board substrate 524. As such, electrical connection and transmission of signals from the elongate sensor 137 to sensor electronics 112 or to a sensor electronics module may be provided.

In examples, the electrical contacts 518a, b may be deposited within the interior chamber 408 through the opening 424 in the upper outer surface 418. The electrical contacts 518a, b may be dispensed in liquid form and then cured within the interior chamber 408 to form an electrical connection with the electrical contacts 211b, 212b of the elongate sensor 137. Electrical isolation between the electrical contacts 518a, b may be provided by the spacing between the electrical contacts 518a, b.

In examples, the electrical contacts 518a, b may be deposited upon the electrical contacts 530a, b of the circuit board substrate 524 and either partially cured or left in a liquid state. The housing 410 may then be inserted over the electrical contacts 518a, b, and then the electrical contacts 518a, b may be fully cured.

Other forms of electrical connection may be provided in examples. For example, conductive pads or pucks, protrusions such as pins, electrically conductive film, magnetic materials may be utilized, among other methods. Other configurations or locations of sockets or receivers may be utilized in examples.

In examples, a sealing member 528 may be provided to seal the electrical connection between the electrical contacts 211b, 212b of the elongate sensor 137 and the electrical contacts 518a, b. The sealing member may further seal the electrical connection between the electrical contacts 518a, b and the electrical contacts 530a, b of the circuit board substrate 524. Further sealing may be provided between the electrical contacts 211b, 212b of the elongate sensor 137 themselves, or any other of the contacts, which may be for purposes of electrical isolation between the contacts.

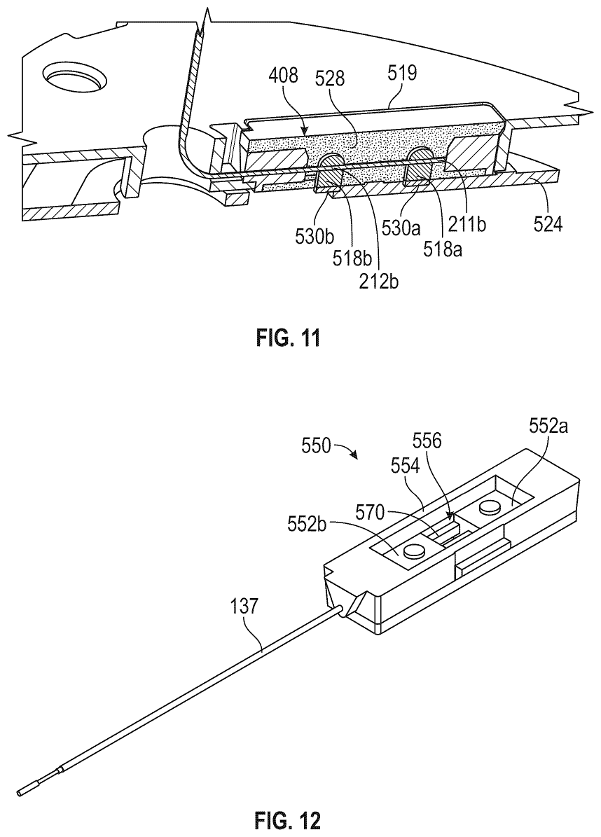

In examples, a fill material, such as a liquid dispensed material may comprise the sealing member 528. Referring to FIG. 11, the liquid dispensed material may be dispensed into the socket 519 and may fill areas around the respective contacts 211b, 212b, 518a, b, 530a, b. The liquid dispensed material may fill the interior chamber 408. The liquid dispensed material may be cured to seal the socket 519 from moisture ingress, which may otherwise undesirably produce an electrical short or other errors (e.g., signal errors) in the system.

In examples, other forms of sealant (e.g., elastomeric bodies) may be utilized as desired.

The configuration of the body that is overmolded upon the elongate sensor 137 may vary in examples.

FIG. 12, for example, illustrates a body overmolded upon the elongate sensor 137 and comprising a housing 550 having one or more electrical contacts 552a, b. The housing 550 may include a frame 554 supporting the one or more electrical contacts 552a, b. The frame 554 may be configured similarly as the housing 410 discussed in regard to FIGS. 4-11 for example.

In examples, the frame 554 may be overmolded upon the elongate sensor 137 in a separate mold or separate mold shot than the one or more electrical contacts 552a, b. For example, the frame 554 may be formed in a shot of injection molding material, to form the frame 554 (which may have an appearance similar to the housing 410 shown in FIG. 4). The frame 554 may be formed in a first mold. The frame 554 may then be provided in a second mold, which may receive another shot of injection molding material to form the one or more electrical contacts 552a, b. The housing 550 may comprise a multi-shot overmolding upon at least a portion of the elongate sensor 137, with the frame 554 being formed in a different shot, and with a different material, than one or more shots that form the one or more electrical contacts 552a, b.

In examples, the order of the shots may be reversed. For example, the one or more shots forming the one or more electrical contacts 552a, b may be first provided, and one or more shots forming the frame 554 may subsequently be provided. The frame 554 accordingly may be formed upon the electrical contacts 552a, b. In examples, multiple molds may be utilized for the formation of the frame 554 and the one or more electrical contacts 552a, b (e.g., a first mold for the frame 554 and a second or third mold for the one or more electrical contacts 552a, b), or a single mold may be utilized with different shot channels for forming both the frame 554 and the one or more electrical contacts 552a, b.

The one or more electrical contacts 552a, b may comprise conductive pucks or other forms of bodies in examples. The one or more electrical contacts 552a, b may be formed from a conductive resin such as a conductive thermoplastic elastomer (TPE). A soft conductive elastomer (e.g., between 55 to 80 shore A hardness, or 40 to 85 shore A hardness, or other ranges) may be utilized. Other materials may be utilized as desired. A conductive TPE may comprise a carbon based TPE or a nickel or silver based TPE, a conductive silicon, or other forms of conductive materials.

The electrical contacts 552a, b may be formed and/or positioned within the interior chamber 556 of the frame 554. The interior chamber 556 may be configured similarly as the interior chamber 408 of the housing 410 unless stated otherwise. The frame 554 includes the features of the housing 410 unless stated otherwise.

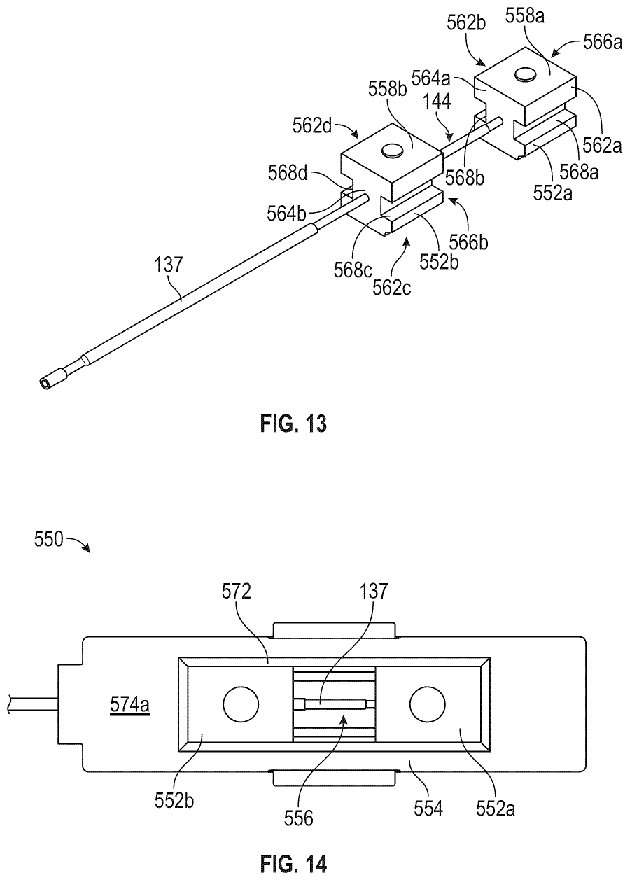

FIG. 13 illustrates the electrical contacts 552a, b in isolation from the frame 554. The electrical contacts 552a, b may be overmolded upon the outer surface 144 of the elongate sensor 137. A first electrical contact 552a may be overmolded upon the contact 211b of the elongate sensor 137. A second electrical contact 552b may be overmolded upon the contact 212b of the elongate sensor 137. The electrical contacts 552a, b may each be overmolded to provide contact with the respective surface of the elongate sensor 137 that the electrical contacts 552a, b are formed upon. A tight fit may be provided that may provide for electrical connection between the contacts 211b, 212b of the elongate sensor 137 and the respective electrical contacts 552a, b. The electrical contacts 552a, b may be spaced from each other for electrical isolation between the electrical contacts 552a, b. The frame 554 created in the initial shot and the shut off conditions provides accurate locations for the conductive contacts to be formed. They are held captive within the structure of frame 554 and anchored within.

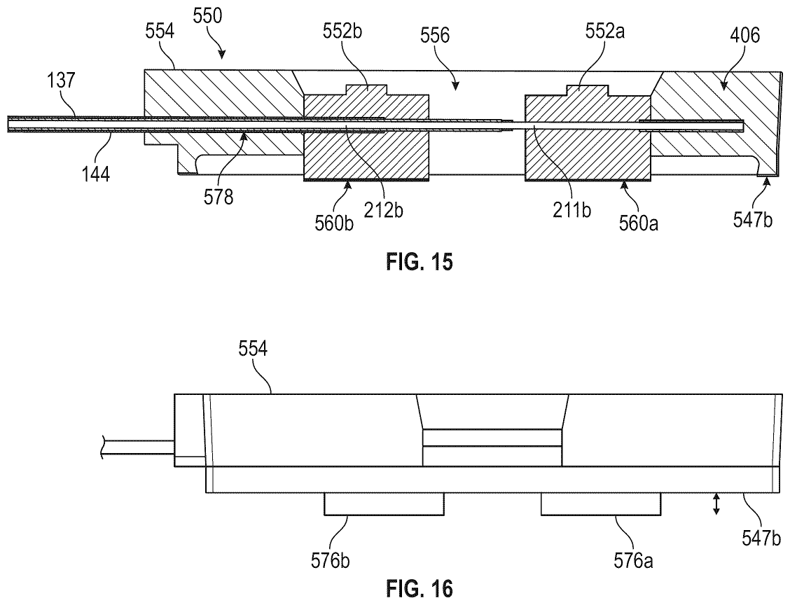

The electrical contacts 552a, b may comprise bodies that are overmolded upon at least a portion of the elongate sensor 137. The bodies may have a volume and the elongate sensor 137 may pass through a central portion of the bodies. The electrical contacts 552a, b, for example, may each have a respective upper outer surface 558a, b, a lower outer surface 560a, b (marked in FIG. 15), respective outer side surfaces 562a, b, c, d facing opposite each other, and respective distal outer end surfaces 564a, b and proximal outer end surfaces 566a, b facing opposite each other. The bodies may each comprise a cuboid shape or may have another shape (e.g., spherical, triangular, etc.) as desired.

The electrical contacts 552a, b may be formed such that the elongate sensor 137 passes through the respective distal outer end surfaces 564a, b of the electrical contacts 552a, b and through the proximal outer end surfaces 566a, b. FIG. 15 illustrates a cross sectional view of the passage of the elongate sensor 137. The tip portion 406 of the elongate sensor 137 may terminate in the frame 554 or may terminate in the electrical contact 552a in examples.

The outer side surfaces 562a, b, c, d may include respective recesses 568a, b, c, d that may be formed during the formation of the electrical contacts 552a, b. The recesses 568a, b, c, d may be formed to mate with securing features in the form of ridges 570 present on the interior side surfaces of the frame 554 (which may correspond to the interior side surfaces 428a, 428b of the housing 410 shown in FIG. 4). The ridges 570 and recesses 568a, b, c, d may comprise securing features to reduce the possibility of the electrical contacts 552a, b from loosening and separating from the frame 554 after formation.

The electrical contacts 552a, b may surround the corresponding contacts 211b, 212b of the elongate sensor 137.

The electrical contacts 552a, b may be formed from a variety of materials, and may comprise a conductive elastomeric material in examples. A conductive elastomeric material may provide for an electrical connection between the contacts 211b, 212b of the elongate sensor 137 and external electrical contacts. In examples, other forms of conductive materials may be utilized for the electrical contacts 552a, b. FIG. 14 illustrates a top view of the housing 550 showing the position of the electrical contacts 552a, b within the frame 554. The electrical contacts 552a, b may be accessible via the opening 572 on the upper outer surface 574a of the frame 554. The electrical contacts 552a, b are shown positioned in the interior chamber 556 of the frame 554 with a spacing between the electrical contacts 552a, b for electrical isolation between the contacts 552a, b.

FIG. 15 illustrates a cross sectional view of the housing 550. The material of the frame 554 may form a seal 578 upon at least a portion of the outer surface 144 of the elongate sensor 137. The seal 578 may prevent moisture from entering the interior chamber 556 of the frame 554.

In examples, the lower outer surfaces 560a, b of the respective electrical contacts 552a, b may protrude from or beyond the lower outer surface 574b of the frame 554. Such a feature may allow for enhanced surface-to-surface connection or outer surface connection between the electrical contacts 552a, b and external electrical contacts (e.g., electrical contacts of a sensor electronics module). The height of the protrusion may vary. FIG. 16, for example, illustrates electrical contacts 576a, b that protrude from the lower outer surface 574b of the frame 554 to a greater distance than shown in FIG. 15. The electrical contacts 576a, b may extend to reach and be compressed against external electrical contacts that may be positioned within windows, recesses, or openings (e.g., windows, recesses, or openings of a socket) as desired.

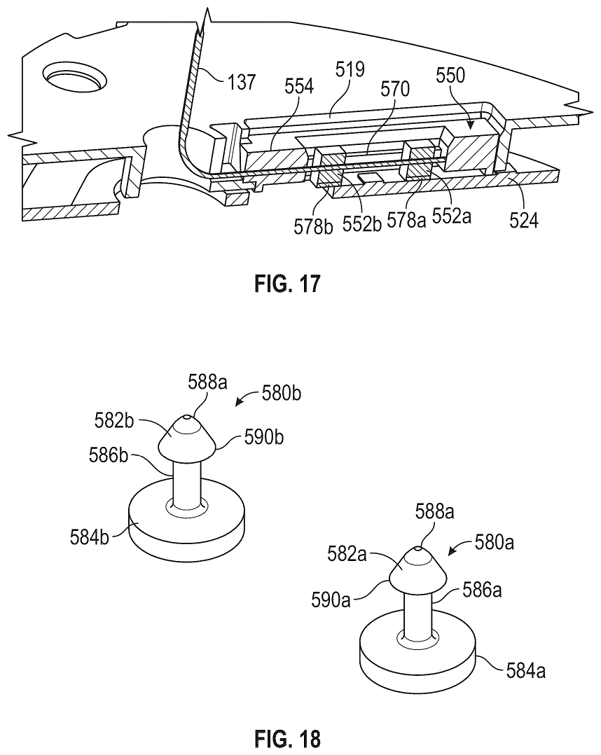

The electrical contacts 552a, b may be configured for an outer surface connection with one or more external electrical contacts. For example, referring to FIG. 17, the housing 550 may be positioned within the socket 519 with the electrical contacts 552a, b forming an outer surface or surface-to-surface connection with the respective external electrical contacts 578a, b. The external electrical contacts 578a, b may comprise pads or pucks or other contacts for outer surface or surface-to-surface connection as desired. A mechanical fit of the frame 554 within the socket 519 may retain the surface contact between the respective contacts 578a, 578b and 552a, b. In a configuration as shown in FIG. 17, a sealing member may be provided that may be similar to the sealing member 528 of FIG. 11 or may comprise another form of sealing member.

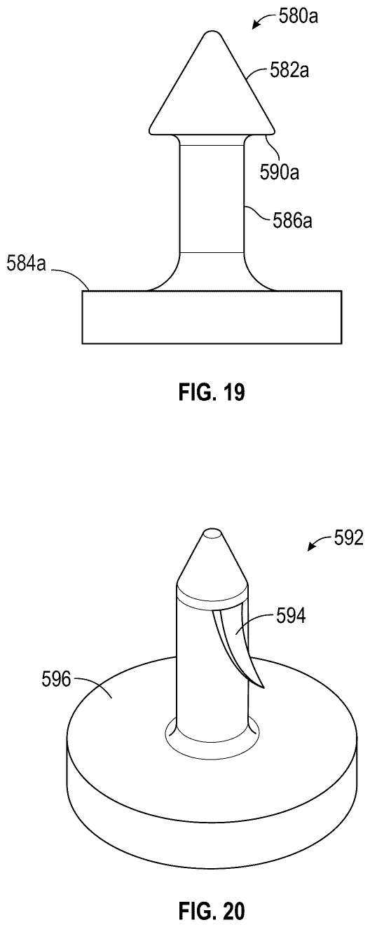

In examples, other forms of mechanical and/or electrical connection may be utilized as desired. One or more conductive protrusions configured to pierce and/or penetrate at least partially into respective conductive members, such as elastomeric conductive members, may be utilized in examples. FIG. 18 illustrates an exemplary set of protrusions that may be utilized in the form of pins 580a, b. Each pin 580a, b may include a respective tip portion 582a, b and a base portion 584a, b. A respective shaft 586a, b may extend between the tip portion 582a, b and the base portion 584a, b. The tip portion 582a, b may include a pointed tip 588a, b that may be configured for penetration as desired. The pointed tip 588a, b may be angled radially inward to form the point of the tip. A conical shape may result (e.g., a conical frustum) or another shape may be utilized as desired. In examples, and referring to the side view of FIG. 19, the tip portion 582a may include a flanged base 590a, b that forms a barb for the respective pin 580a, b. The shaft 586a, b may have a lesser diameter than the respective flanged base 590a, b.

The base portion 584a, b may comprise a plate or terminal for electrical connection to an electrical trace or circuit board substrate, which may be an electrical trace or circuit board substrate of the sensor electronics 112 or sensor electronics module. The base portion 584a, b may be electrically connected (e.g., soldered or another form of electrical connection) as desired.

The pins 580a, b may be formed in a variety of methods. A method may include forming the pins 580a, b with a lathe that may cut the pattern or profile of the pins 580a, b. The pattern or profile may be cut from an elongate wire that may be cut to a size (e.g., height of the respective pins 580a, b). Lathe processing may be utilized for any form of pin disclosed herein.

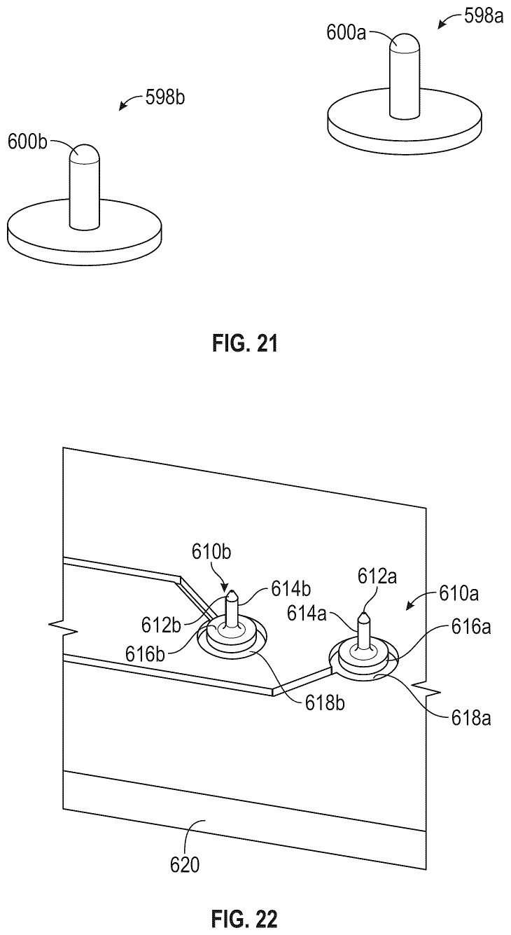

Other forms of pins may be utilized in examples. FIG. 20 illustrates a pin 592 having a barb 594 in the form of an arm extending towards the base portion 596 of the pin 592. FIG. 21 illustrates an example of pins 598a, b lacking a barb and having rounded tip portions 600a, b.

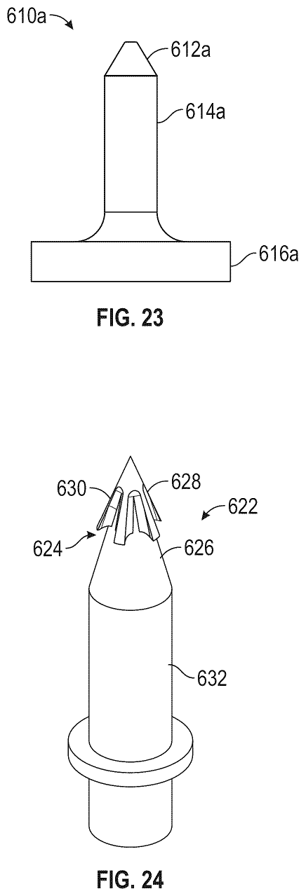

FIG. 22 illustrates an example of pins 610a, b lacking a barb, and having respective angled tip portions 612a, b. The angled tip portions 612a, b may angle radially inward to form the point of the tip. The shafts 614a, b may have cylindrical shapes or may have other shapes as desired. The base portions 616a, b are shown electrically connected to respective electrical traces 618a, b of a circuit board substrate 620. FIG. 23 illustrates a side view of the pin 610a.

FIG. 24 illustrates an example of a pin 622 that may be utilized and includes a tip portion 624 having an angled smooth surface 626 extending distally to a barbed tip 628 having grooves or fluting on an outer surface 630. By such a configuration, the tip portion 624 can be provided with increased surface area and thus provide increased surface-to-surface contact with the conductive elastomer. The angled smooth surface 626 may angle radially inward from the outer diameter of the shaft 632. The shaft 632 may have a greater or equal diameter than the diameter of the tip portion 624.

Various other forms of protrusions may be utilized as desired. In examples, the electrical contacts 552a, b may be configured to be penetrated by one or more external electrical contacts for an electrical connection. The protrusions may comprise the external electrical contacts that penetrate the electrical contacts 552a, b. The electrical contacts 552a, b may comprise elastomeric and compliant bodies, which may allow for penetration. The electrical contacts 552a, b may surround and may conform to a shape of the penetrating protrusions. The contact between the electrical contacts 552a, b and the protrusions may allow for the electrical connection between the electrical contacts 552a, b and the protrusions and accordingly between the contacts 211b, 212b of the elongate sensor 137 and the protrusions.

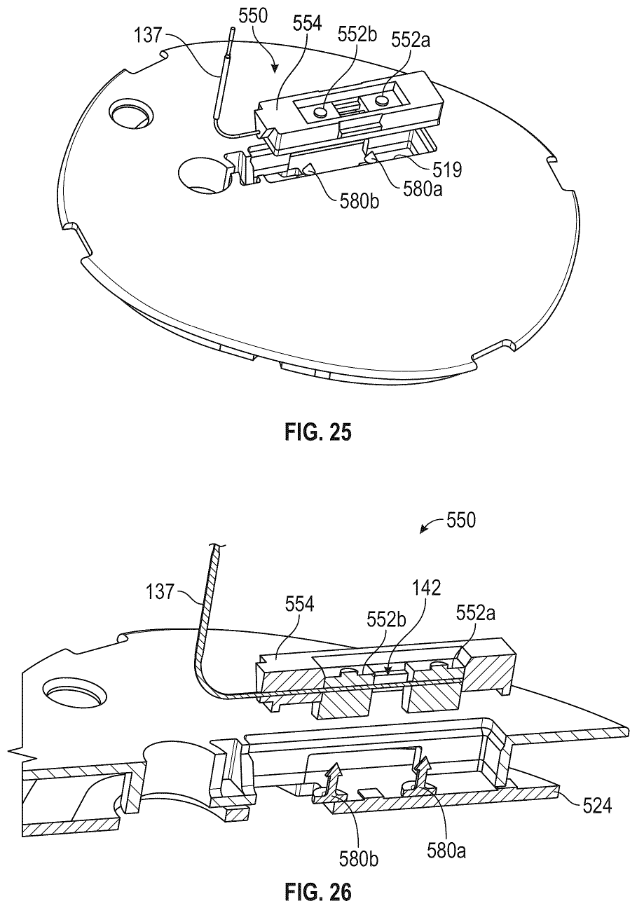

FIG. 25, for example, illustrates the housing 550 approaching a socket 519. Electrical contacts in the form of pins 580a, b are present in the socket 519 for mechanically engaging with and electrically connecting to the electrical contacts 552a, b. FIG. 26 illustrates a cross sectional view of the approach of the housing 550 towards the pins 580a, b. Insertion in a direction transverse or perpendicular to the length of the proximal end portion 142 of the elongate sensor 137, and the housing 550, may be provided.

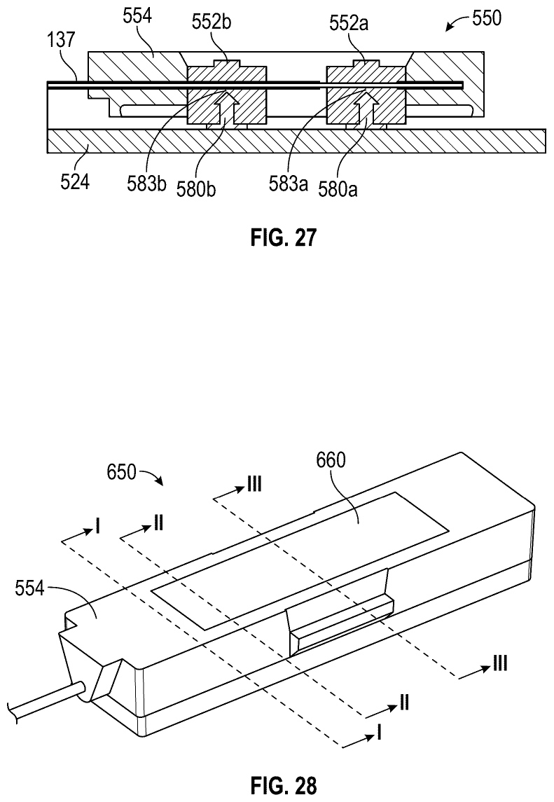

FIG. 27 illustrates an exemplary connection between the pins 580a, b and the electrical contacts 552a, b. The pins 580a, b may serve to anchor the housing 550 to the circuit board substrate 524, which may be utilized in combination with or in lieu of mechanical anchoring provided by the frame 554.

The pins 580a, b may penetrate the electrical contacts 552a, b for a distance, yet may be spaced from the elongate sensor 137 with a respective gap 583a, b. The gap 583a, b may be provided to avoid direct contact between the tips of the pins 580a, b and the elongate sensor 137, which may be undesirable. In examples, direct contact may be provided. The size of the gap 583a, b may be set based on the geometry of the pins 580a, b and the position of the elongate sensor 137. The position of the elongate sensor 137 may be determined based on the size of the housing 550 and the distance of the lower outer surface of the housing 550 from the elongate sensor 137. Electrical connection between the contacts 211b, 212b of the elongate sensor 137 and the electronics of the circuit board substrate 524 may result (which may comprise sensor electronics).

In examples, other forms of pins or protrusions may be utilized as desired.

In examples, the pins or protrusions may be positioned on the housing 550 and may extend from the housing for penetration of electrical contacts external of the housing 550. For example, the configuration of electrical contact shown in FIG. 27 may be inverted, with protrusions or pins 580a, b extending from (e.g., formed with or coupled to) the contacts 552a, b and configured to pierce and/or penetrate one or more electrical contacts disposed in or on other structure (e.g., a socket 519 as shown in FIG. 25) in an on-skin device. Offsets in the x- and y-directions of the electrical contacts may prevent engagement with the elongate sensor and allow a deeper penetration into the conductive elastomer.

A sealing member may be provided that may be similar to the sealing member 528 of FIG. 11. In examples, other forms of sealing members may be utilized.

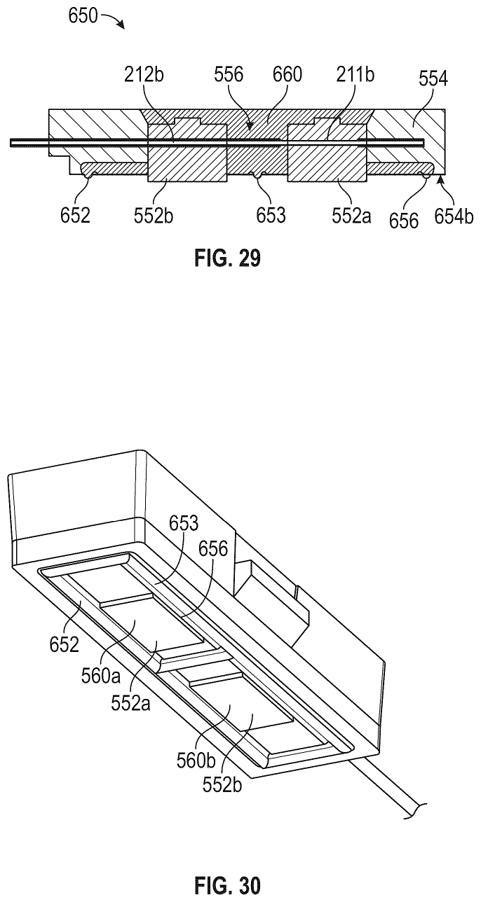

FIG. 28, for example, illustrates a housing 650 that includes a sealing member 652 (shown in FIGS. 29 and 30). The sealing member 652 may be disposed at the lower outer surface 654b of the housing 650. The housing 650 includes the features of housing 550 unless stated otherwise.

Referring to FIG. 30, the sealing member 652 may seal the electrical connection between the electrical contacts 552a, b and external electrical contacts (which may comprise any form of external electrical contact disclosed herein). The sealing member 652 may comprise one or more ribs 653 and/or grooves 656 for forming a seal with a surface. The one or more ribs 653 and/or grooves 656 may form a perimeter about each or both of the electrical contacts 552a, b, and particularly the lower outer surfaces 560a, b of the electrical contacts 552a, b. The one or more ribs 653 and/or grooves 656 may form a ring about each or both of the electrical contacts 552a, b to prevent moisture ingress interior of the ring (in which the electrical contacts 552a, b are positioned). The lower outer surfaces 560a, b of the electrical contacts 552a, b may protrude from the sealing member 652 as shown in FIG. 30. Other forms of sealing members may be utilized as desired. The sealing member 652 may comprise an elastomeric material for forming the seal, and may be non-conductive in examples. A soft compressible elastomeric material (e.g., between 20 to 60 shore A hardness, or between 10 to 70 shore A hardness, or other ranges) may be utilized. The sealing member 652 may be configured to form a face seal.

Referring to FIG. 29, the sealing member 652 may comprise all or a portion of a fill material 660 that may fill the interior chamber 556 of the frame 554. The fill material 660 may comprise a sealing member that may seal the interior chamber 556 of the frame 554. For example, the fill material 660 may be non-conductive and may electrically insulate the contacts 552a, b from each other. The fill material 660 may electrically insulate the contacts 211b, 212b of the elongate sensor 137 from each other. The fill material 660 may prevent moisture ingress into the interior chamber 556 of the frame 554. The sealing member 652 may be integral with the fill material 660 in examples.

The body comprising the housing 650 may encapsulate the proximal end portion of the elongate sensor 137.

In examples, the fill material 660 may comprise a body that is overmolded upon the elongate sensor 137. The fill material 660 may be overmolded upon the elongate sensor 137 in a separate mold or separate mold shot than the frame 554 and/or the one or more electrical contacts 552a, b. For example, the frame 554 may be formed in a shot of injection molding material, to form the frame 554 (which may have an appearance similar to the housing 410 shown in FIG. 4). The frame 554 may be formed in a first mold. The frame 554 may then be provided in a second mold, which may receive another shot of injection molding material to form the one or more electrical contacts 552a, b. The frame 554 and electrical contacts 552a, b may be provided in a third mold, which may receive another or third shot of injection molding material to form the fill material 660 and the sealing member 652. The housing 650 may comprise a multi-shot overmolding upon at least a portion of the elongate sensor 137, with the frame 554 being a different shot than one or more shots that form the one or more electrical contacts 552a, b and the fill material 660 and/or sealing member 652 being a different shot than the shots that form the frame 554 and the one or more electrical contacts 552a, b.

In examples, the order of the shots may be reversed. For example, the one or more shots forming the fill material 660 and/or sealing member 652 may be provided first, and then the one or more electrical contacts 552a, b may be provided with one or more shots, and then one or more shots forming the frame 554 may subsequently be provided. Other variations in the order or sequence may be provided as desired. In examples, multiple molds may be utilized for the formation of the frame 554 and the one or more electrical contacts 552a, b (e.g., a first mold for the frame 554 and a second or third mold for the one or more electrical contacts 552a, b) and another mold for the fill material 660 or sealing member 652, or a single mold may be utilized with different shot channels for forming both the frame 554 and the one or more electrical contacts 552a, b and the fill material 660 and/or sealing member 652. The material of the fill material 660 and/or sealing member 652 may comprise a non-conductive thermoplastic elastomer (TPE) or another form of material as desired.

The overmolding processes disclosed herein may occur prior to a membrane coating process, calibration, and/or testing steps and/or prior to a mechanical coupling, electrical connection, and/or sealing steps.

The frame 554 may be formed with features configured to support the fill material 660 and/or sealing member 652. For example, referring to the cross sectional view of FIG. 31 (taken along line I-I in FIG. 28), the frame 554 may include a cavity 664 for the sealing member 652 to radially protrude into. The radial protrusion may comprise a radial seal that may prevent the ingress of moisture into the interior chamber 556 of the frame 554.

FIG. 32 illustrates a cross sectional view along the line II-II in FIG. 28. The cavity 664 is shown to continue along a position adjacent the electrical contact 552b. A supporting surface 666 of the frame 554 may support the sealing member 652 from compression in a direction inward towards the interior of the housing 650.

FIG. 33 illustrates a cross sectional view along line III-III in FIG. 28. The sealing member 652 is shown to be integral with the fill material 660 that fills the space between the contacts 211b, 212b of the elongate sensor 137.

In examples, other forms of sealing may be provided. For example, a sealing member may be provided on the circuit board substrate 524 or another form of substrate and may seal with a portion of a housing of an analyte sensor system or module.

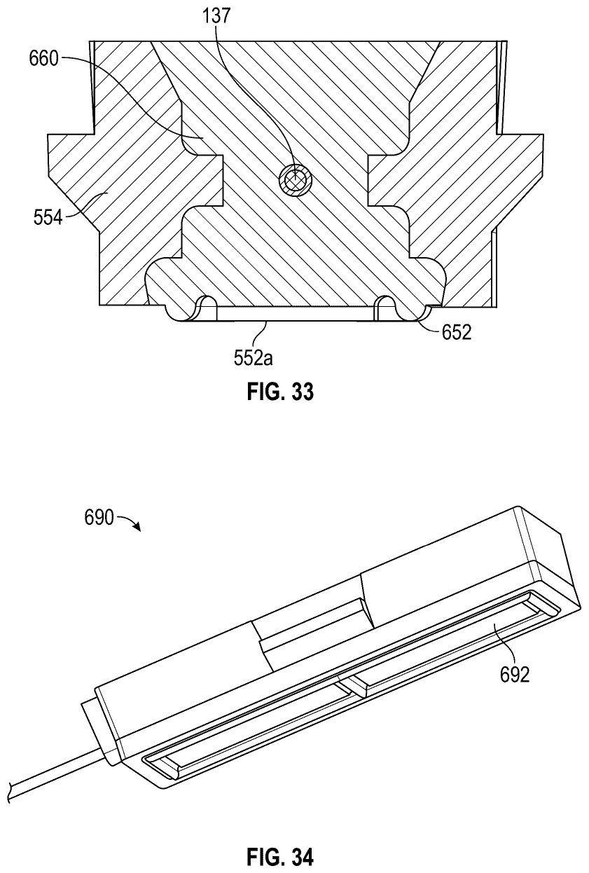

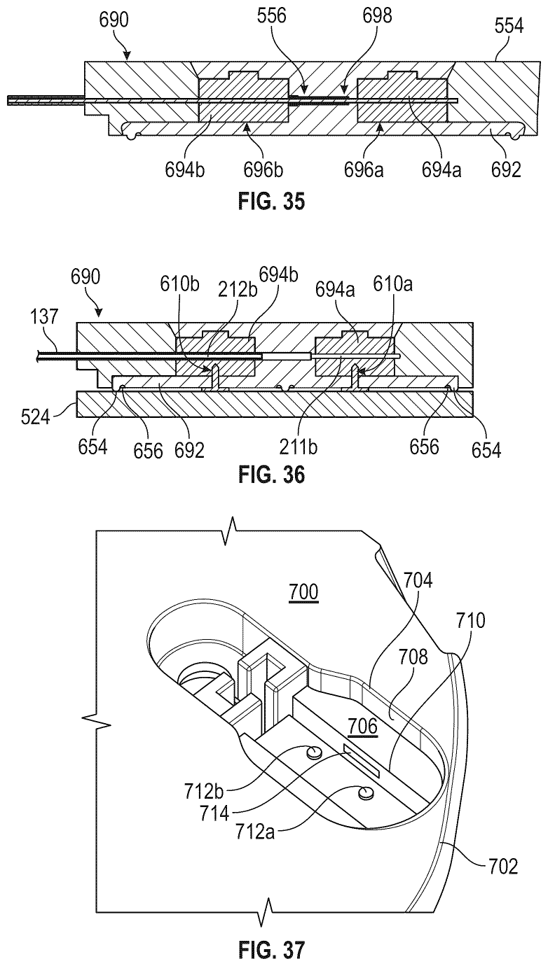

FIG. 34 illustrates an implementation of a housing 690 including a sealing member 692 that covers the electrical contacts 694a, b (marked in FIG. 35). The housing 690 includes the features of the housing 650 unless stated otherwise.

The sealing member 692 covers the lower outer surfaces 696a, b of the electrical contacts 694a, b. The electrical contacts 694a, b are configured similarly as the electrical contacts 552a, b unless stated otherwise. A height of the electrical contacts 694a, b may be reduced from the height of the electrical contacts 552a, b such that the sealing member 692 may cover the lower outer surfaces 696a, b of the electrical contacts 694a, b. As shown in FIG. 34, the sealing member 692 comprises a continuous surface that extends over the lower outer surfaces 696a, b of the electrical contacts 694a, b.

The sealing member 692 may be formed in a similar manner as the sealing member 652 shown in FIG. 30. Molding processes may be utilized to form the sealing member 692 in a similar manner as the sealing member 652, yet with the sealing member 692 covering the lower outer surfaces 696a, b of the electrical contacts 694a, b. The sealing member 692 may be integral with fill material 698 (marked in FIG. 35) that may fill the interior chamber 556 of the frame 554.

The sealing member 692 may be configured to be penetrated by one or more external electrical contacts for an electrical connection with the electrical contacts 694a, b. For example, the sealing member 692 may be made of an elastomeric material that may be compliant and penetrable by a protrusion (e.g., a pin). The material of the sealing member 692 may seal around the protrusion to prevent moisture ingress therebetween.

FIG. 36, for example, illustrates pins 610a, b having penetrated through the sealing member 692 for electrical connection with the electrical contacts 694a, b. The sealing member 692 may seal about the pins 610a, b to prevent moisture from entering the penetration hole caused by the pins 610a, b. As such, electrical shorting or other errors in the connection between the pins 610a, b and the electrical contacts 694a, b (and correspondingly the electrical contacts 211b, 212b of the elongate sensor 137) may be reduced. Other features of the sealing member 692 (e.g., one or more ribs 653 and/or grooves 656) may be utilized in combination if desired. The one or more ribs 653 and/or grooves 656 may prevent moisture ingress to the base portion of the pins 610a, b and the electrical connection to the circuit board substrate 524.

The sealing member 692 in examples, may comprise a resilient material that may be a self-sealing or self-healing material that may provide a seal of a penetration hole even upon the pins 610a, b being withdrawn. As such, the housing 690 may be removed from the pins 610a, b and the penetration holes may be sealed to maintain the electrical integrity of the interior of the housing 690 and particularly the electrical contacts 694a, b. Benefits may result from such a feature. For example, the housing 690 may be coupled to a testing apparatus in which pins penetrate the sealing member 692 for electrical connection with the electrical contacts 694a, b. Force activated pogo pins, for example pogo pins with piercing tip geometries, can be utilized. Electrical testing of the elongate sensor 137 may occur, with reduced possibility of moisture damage to the analyte sensor system due to the seal provided by the sealing member 692. The housing 690 may then be removed from the pins of the testing apparatus and either placed in another testing apparatus or assembled with the sensor electronics or sensor electronics module. Other pins may pass through the sealed septum of the sealing member 692. The integrity of the electrical connection with the other pins may be maintained by the seal provided by the sealing member 692. Multiple punctures of the sealing member 692 may occur with the sealing properties of the sealing member 692 being maintained.

In testing processes, the analyte sensor systems may be coupled to fixtures and moved from station to station for various calibration and/or testing steps in which the sensors interface with electrical measurement equipment. The housings may protect and seal the elongate sensor during such processes. The overmolding processes may facilitate handling of the sensor during both pre-assembly and assembly processes, and can seal the proximal end portion of the elongate sensor against ingress of moisture.

The housing 690 may comprise a modular, self-sealing device for use in handling, testing, and assembly of the analyte sensor system. The housing 690 may protect the proximal end portion of the elongate sensor 137 from moisture and other damage, whether through the testing, handling, or assembly processes. In examples, the housing 690 may be submerged in fluid, yet the interior of the housing 690 may remain sealed from moisture ingress.

The modular configuration of housings disclosed herein may allow the housings to be mechanically and/or electrically coupled with a variety of configurations of sockets, electrical contacts, and/or other components of on-skin wearable medical devices. The modular housings may be coupled to a variety of different forms of on-skin wearable medical devices. A single housing may be configured to mechanically and/or electrically couple with a variety of different configurations of receivers, electrical contacts, and/or other components of on-skin wearable medical devices.

FIG. 37, for example, illustrates a variation in the configuration of the socket 519 shown in FIG. 9. An external bottom surface 700 of an on-skin wearable housing 702 includes a recess 704. The recess 704 has an external bottom surface 706 and includes one or more side walls 708 bounding the volume of the recess 704. The external bottom surface 706 of the recess 704 has a socket 710 in the form of a recess in the external bottom surface 706. The socket 710 may be formed in the wall of the wearable housing 702. The socket 710 may include electrical contacts 712a, b and one or more mating features 714 for mating with a housing of an analyte sensor system as disclosed herein. The socket 710 may be positioned upon a circuit board substrate 524 (marked in FIG. 39), with the electrical contacts 712a, b positioned upon the circuit board substrate 524.

The socket 710 may include one or more interior side walls bounding the volume or cavity of the socket 710 and may be shaped to receive a housing of an analyte sensor system as disclosed herein. The mating features 714 may be positioned upon the interior side walls. The electrical contacts 712a, b may be positioned upon a bottom surface of the socket 710.

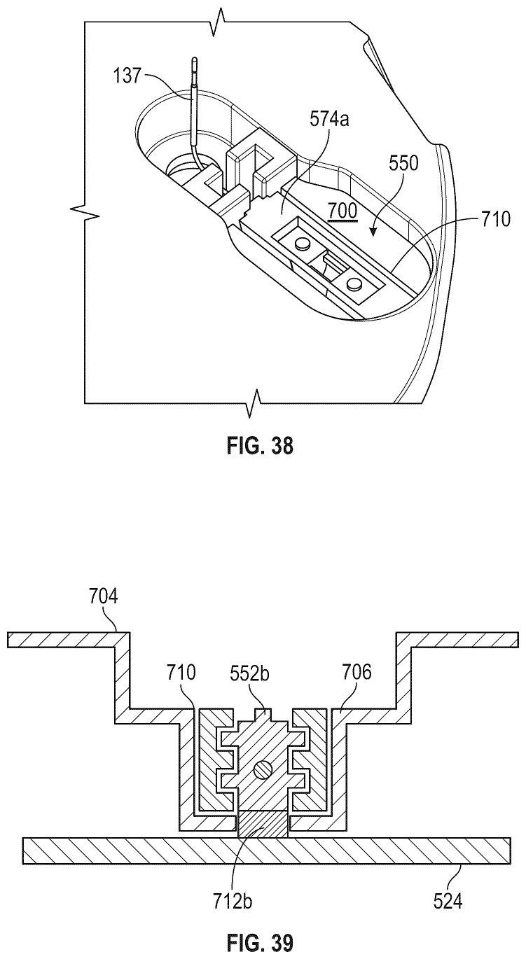

FIG. 38 illustrates an analyte sensor module or housing 550 inserted into the socket 710. The upper outer surface 574a of the housing 550 may be flush with the external bottom surface 706 of the recess 704 after insertion, or another configuration may be utilized in examples. FIG. 39 illustrates a cross sectional view of the mating configuration. An encapsulant may fill the recess 704, or another form of sealing member may be utilized as desired. Any other form of housing of an analyte sensor system disclosed herein may be utilized.

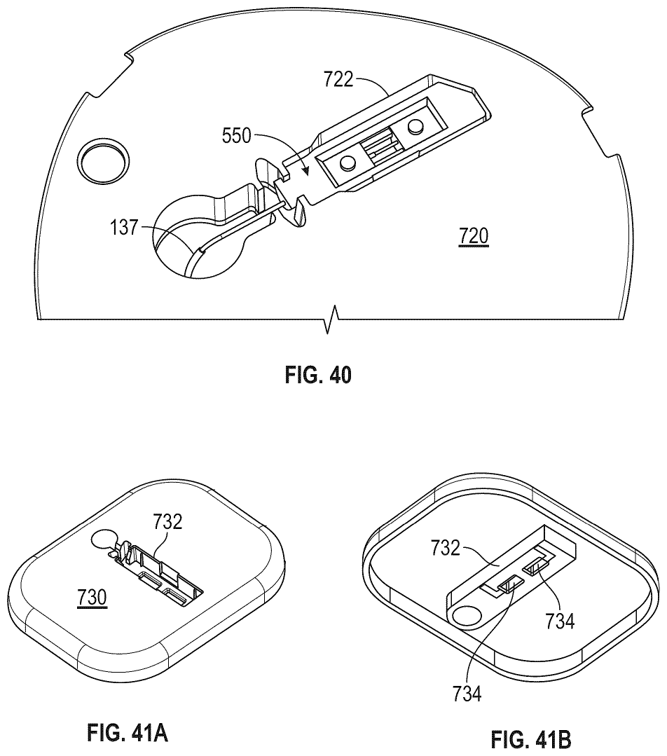

Other mating locations may be utilized. FIG. 40, for example, illustrates a configuration in which the housing 550 may be inserted into an interior bottom surface 720 of a wearable housing of an on-skin wearable medical device. The socket 722 may be positioned upon the interior bottom surface 720. The socket may be formed in a wall of the wearable housing. The elongate sensor 137 may extend distally from the interior bottom surface 720 for insertion into skin of a host.

The housing 550, or any other housing of an analyte sensor system disclosed herein, may be disposed internal of a housing of an on-skin wearable medical device in a configuration shown in FIG. 40. In examples, other locations of receiving portions or sockets (e.g., in an upper or top housing or internal housing) may be utilized.

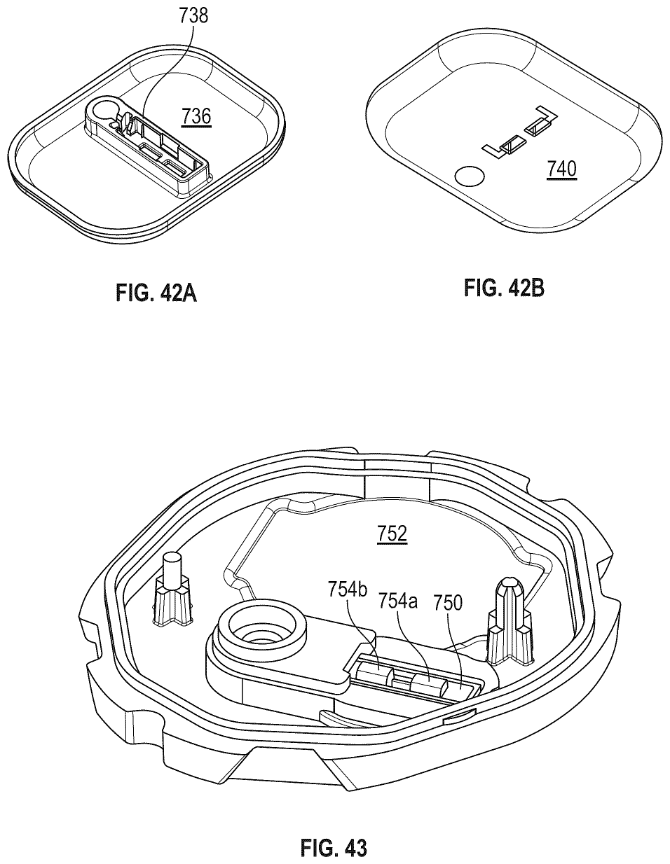

FIGS. 41A-42B illustrate examples of sockets for the analyte sensor system formed in a wall of material forming a wearable housing of the on-skin wearable medical device.

FIG. 41A, for example, illustrates an external upper or top surface 730 of a wearable housing for an on-skin wearable medical device. The receiving portion or socket 732 is formed in the external top surface 730 and comprises a recess in the external top surface 730. FIG. 41B illustrates an opposite side view showing the socket 732 having one or more openings 734 for electrical connection with the analyte sensor system.

FIG. 42A illustrates an internal top surface 736 of a wearable housing for an on-skin wearable medical device. The socket 738 is formed in the internal top surface 736 and comprises a raised portion of the internal top surface 736. Electrical access to the analyte sensor system may be provided from above the socket 738 shown in FIG. 42A. FIG. 42B illustrates an opposite side view showing the external top surface 740 of the wearable housing.

The sockets 732, 738 shown in FIGS. 41A-42B may be formed from the material of the wearable housing for an on-skin wearable medical device. In examples, the sockets 732, 738 may be separately formed and applied to or assembled with the on-skin wearable medical device. In examples, other forms of receiving portions of on-skin wearable medical device may mechanically and/or electrically coupled with analyte sensor systems as disclosed herein.

FIG. 43 illustrates an implementation of a housing 750 of an analyte sensor system as disclosed herein positioned on an internal bottom surface 752 of a wearable housing for an on-skin wearable medical device. The electrical contacts 754a, b of the housing 750 may remain exposed for connection to external electrical contacts.

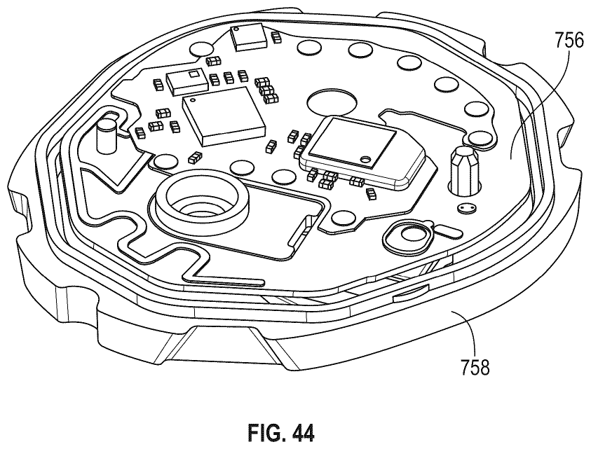

A circuit board substrate 756 as shown in FIG. 44 may be applied to the electrical contacts 754a, b of the housing 750 for electrical connection. The circuit board substrate 756 may include sensor electronics 112 or sensor electronics module. An upper or top housing may be applied to the lower or bottom housing 758 shown in FIG. 44 to enclose the components therein. Various forms of sealing (e.g., sealing members of housings disclosed herein, encapsulating fill material, ultrasonic welding, laser welding, other forms of weld lines, among others) may be utilized to seal the interior of the wearable housing of the on-skin wearable medical device.

In examples, a socket for an analyte sensor system may be formed using surface mount technology (SMT). The socket may be formed upon a circuit board substrate. The socket may be positioned upon the circuit board substrate for receiving the housing of the analyte sensor system.

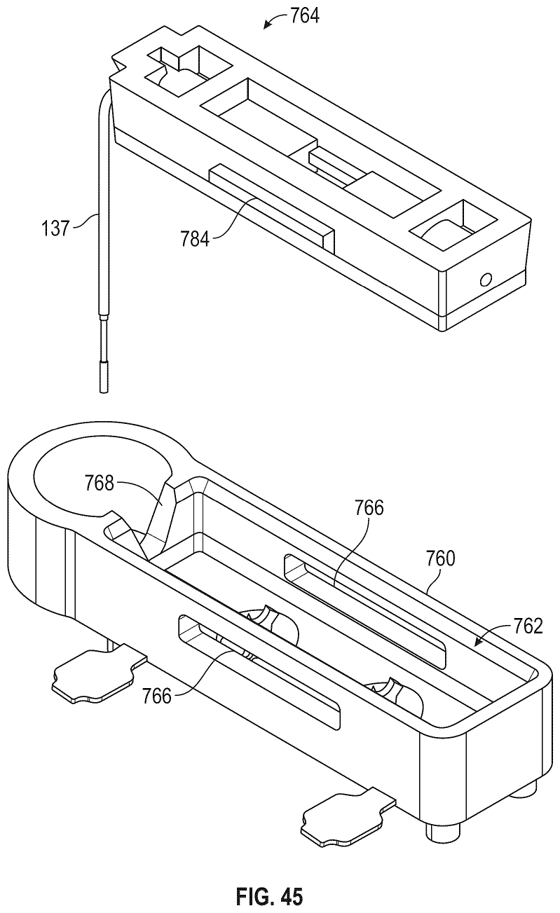

FIG. 45, for example, illustrates an implementation of a receiving portion in the form of a socket 760 that would be positioned upon a circuit board substrate. The socket 760 may be formed with SMT. The socket 760 may be configured similarly as other forms of sockets disclosed herein, and may include a cavity 762 for receiving the housing 764 of an analyte sensor system and may include one or more mating features 766, 768 for mating with the housing 764 in manners disclosed herein. The housing 764 may be configured similarly as other forms of housings of analyte sensor systems disclosed herein. In examples, the socket 760 is an injection molded component with a metal lead frame integrated within the material of the socket (e.g., plastic) that acts as a mechanical/electrical connection to a printed circuit board (PCB) with solder or alternative conductive material.

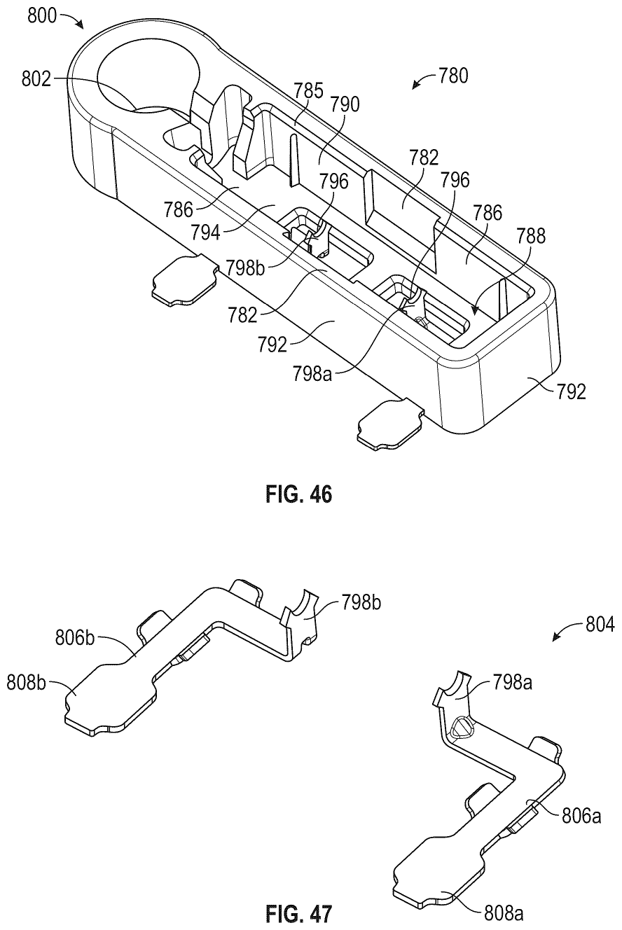

FIG. 46 illustrates a variation of the socket of FIG. 45 comprising a socket 780 having a different form of mating features 782. The mating features 782 may provide an interference fit with corresponding mating features 784 of the housing 764.

The socket 780 may include an upper opening 785 and one or more side walls 786 bounding the interior chamber 788 of the socket 780. The one or more side walls 786 may each include an inner surface 790 facing the interior chamber 788 and an outer surface 792 facing away from the interior chamber 788. The socket 780 may be shaped to receive the housing 764.

A bottom surface 794 or lower surface of the socket 780 may include one or more openings 796 for electrical contacts 798a, 798b to pass through. An end portion 800 of the socket 780 may include an opening 802 for the elongate sensor 137 to pass through.

FIG. 47 illustrates a perspective view of a lead frame 804 that may be utilized with the socket 780. The lead frame 804 may include electrical conduits 806a, b that may connect with the respective electrical contacts 798a, b. The electrical conduits 806a, b may include electrical terminals 808a, b that may electrically connect to other components of a circuit board substrate and/or sensor electronics or a sensor electronics module.

The electrical contacts 798a, b may be configured to pass through the openings 796 of the socket 780. The tips of the electrical contacts 798a, b may include crescent shapes or concave cut-outs that may reduce the possibility of direct contact with a circular elongate sensor 137 yet allow for closer proximity to the elongate sensor 137.

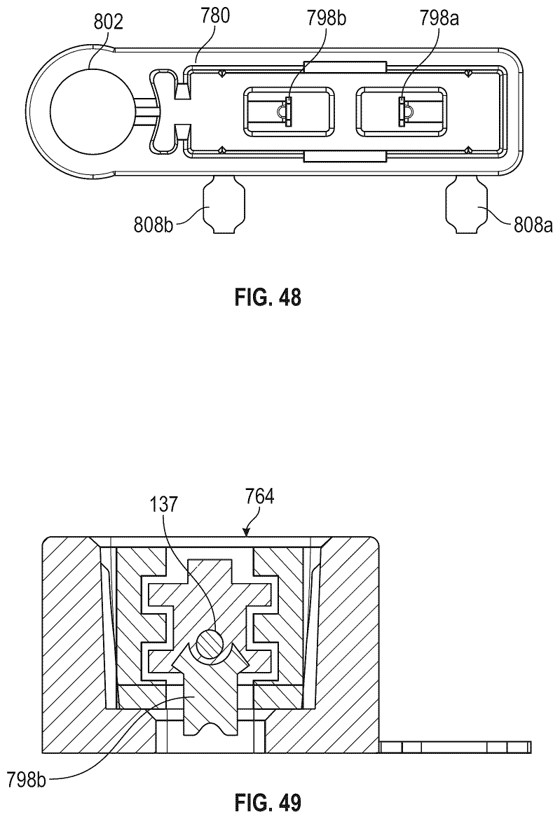

FIG. 48 illustrates a top view of the socket 780. FIG. 49 illustrates a side cross sectional view of the housing 764 positioned within the socket 780. The crescent shape of the electrical contact 798b is shown to contour to a shape of the elongate sensor 137, without the electrical contact 798b directly contacting the elongate sensor 137. A gap may be provided between the electrical contact 798b and the elongate sensor 137.

The socket 780 and the lead frame 804 may be formed with surface mount technology (SMT), or in examples, may be separately formed and bonded to another substrate such as a circuit board substrate.

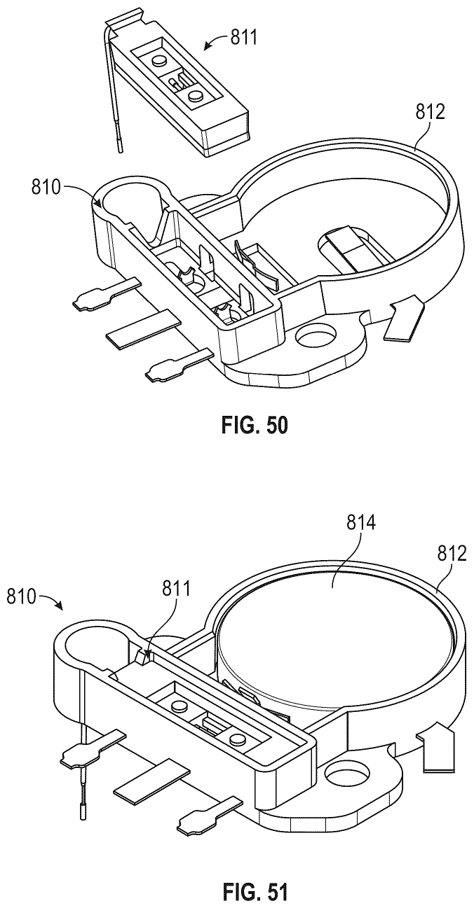

FIG. 50 illustrates a configuration of a socket 810 configured similarly as the socket 780 shown in FIG. 46, yet including a power source carriage 812 (e.g., a battery socket) integral with the socket 810. The socket 810 and power source carriage 812 may be integral with each other, and may be formed using surface mount technology (SMT), or in examples, may be separately formed and bonded to another substrate such as a circuit board substrate.

FIG. 51 illustrates an analyte sensor system 811 and a battery 814 mated with the respective socket 810 and power source carriage 812.

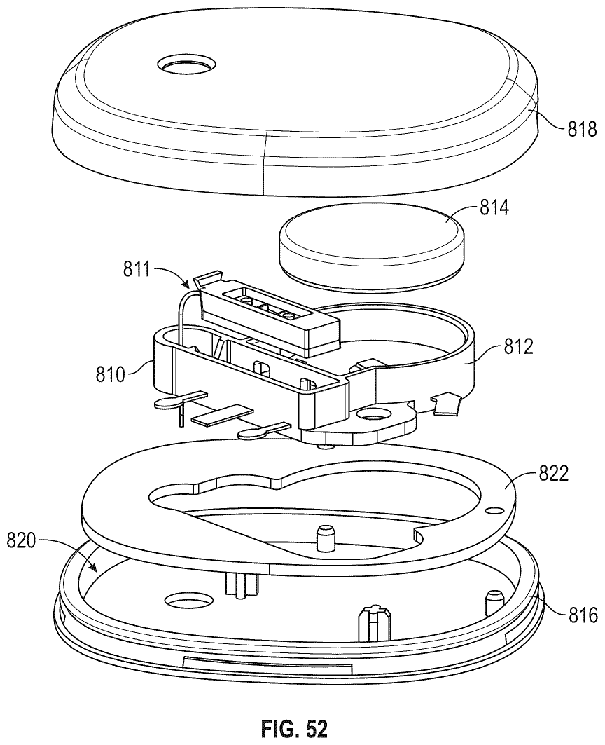

Additional exemplary assembly steps are shown in FIGS. 52-54.

FIG. 52 illustrates an exploded view showing a lower or bottom housing 816 and an upper or top housing 818 configured to form an interior chamber 820 for receiving the socket 810, power source carriage 812, and the analyte sensor system 811. The bottom housing 816 and top housing 818 together may form a wearable housing 819 (marked in FIG. 54) of the on-skin wearable medical device. The socket 810 and power source carriage 812 may be positioned between the bottom housing 816 and the top housing 818. A circuit board substrate 822 comprising sensor electronics or a sensor electronics module may further be provided within the interior chamber 820.

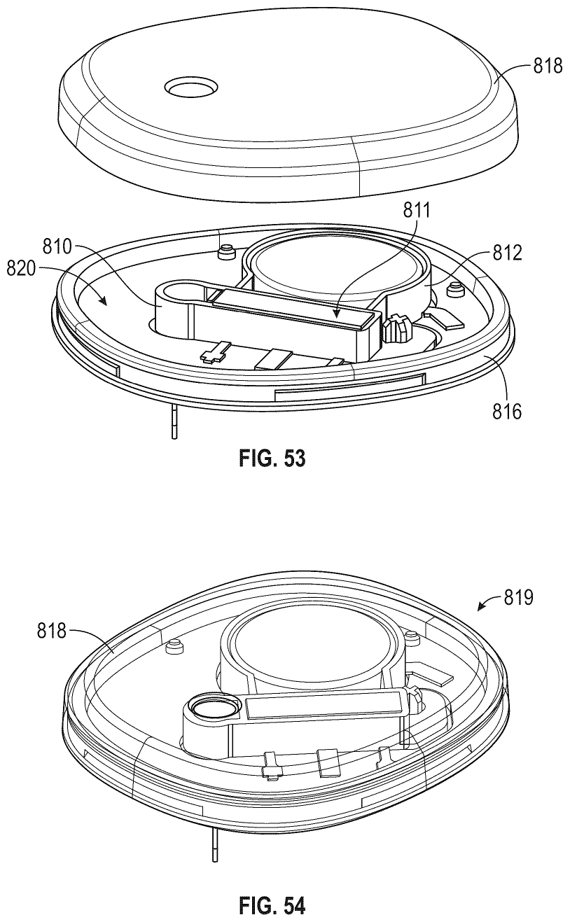

FIG. 53 illustrates that the socket 810 and other components may be positioned within the interior chamber 820 of the wearable housing 819. FIG. 51 illustrates that the top housing 818 may be applied to seal the interior of the wearable housing 819. If desired, an encapsulant may fill the interior chamber 820 for additional sealing of the interior of the wearable housing 819.

In examples, a socket may comprise a flexible body, which be formed from, for example, an elastomeric material. The flexible body may be configured to flex to accommodate movement of the host's body that may produce forces against the on-skin wearable medical device and socket. Further, sockets as disclosed herein may be configured for lamination to a substrate.

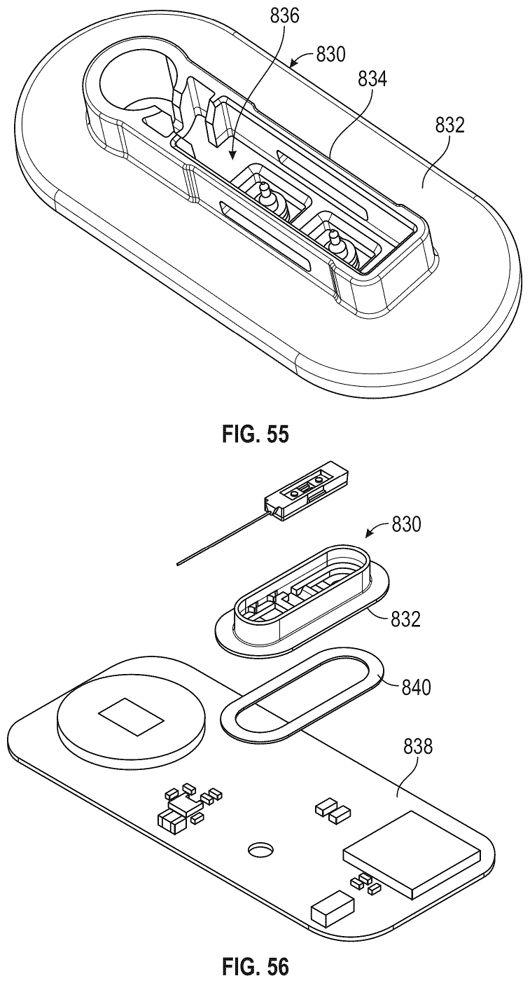

FIG. 55, for example, illustrates a configuration of socket 830 that may be configured similarly as the socket 780 unless stated otherwise. The socket 830 may include a flanged portion 832 that may protrude radially outward from the walls 834 defining the interior chamber 836 of the socket 830. The flanged portion 832 may comprise a disk having a surface area for coupling to a substrate (e.g., a circuit board substrate).

For example, referring to FIG. 56, the socket 830 is shown in an exploded view for application to a flexible circuit board substrate 838. The socket 830 may be applied to the flexible circuit board substrate 838 in a variety of manners. In examples, the flanged portion 832 of the socket 830 may be coupled to the flexible circuit board substrate 838 utilizing an adhesive 840. The adhesive 840 may comprise a film such as an adhesive tape in examples. Other forms of adhesive may be utilized in examples (e.g., a dispensed adhesive that may be cured utilizing a variety of methods).

The socket 830 may be applied to the flexible circuit board substrate 838 in a lamination process. For example, layers comprising the flexible circuit board substrate 838, the adhesive 840, and the socket 830 may be applied upon each other and pressed together. In examples, reel-to-reel processes may be utilized to couple the flexible circuit board substrate 838, the adhesive 840, and the socket 830 together. A resulting configuration is represented in FIG. 57.

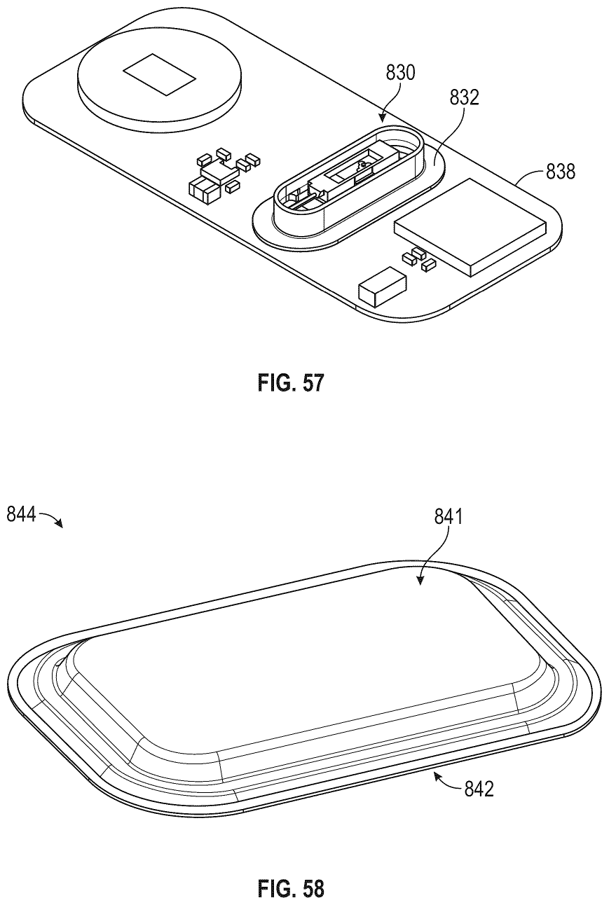

One or more additional layers may be provided to form an upper or top housing body and a lower or bottom housing body. FIG. 58, for example, illustrates a top housing body 841 and a bottom housing body 842 laminated together to form on-skin wearable housing 844. The on-skin wearable housing 844 may be flexible to accommodate movement of a host's skin upon application.

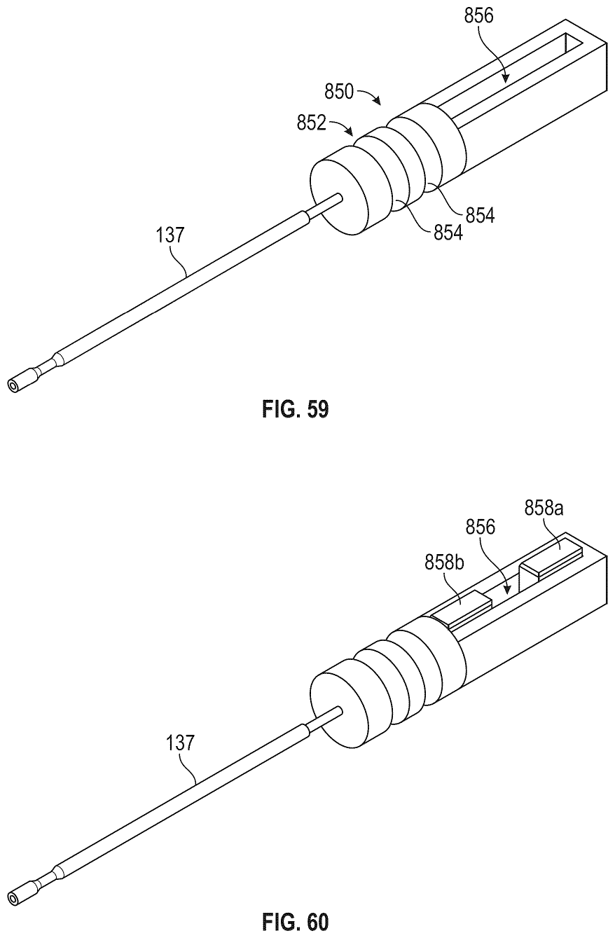

Variations in the configuration of the analyte sensor system may be provided. FIG. 59 illustrates an example in which a housing 850 may be formed having a distal end portion 852 with a circular outer profile. The distal end portion 852 may include grooves 854 for receiving sealing members such as o-rings for sealing with a structure that the housing 850 is mated with. An interior chamber 856 of the housing 850 may be configured for access to the contacts 211b, 212b of the elongate sensor 137. The housing 850 may be overmolded upon the elongate sensor 137.

FIG. 60 illustrates a variation of the housing 850 of FIG. 59, in which electrical contacts 858a, b are disposed within the interior chamber 856. The electrical contacts 858a, b may be overmolded upon the elongate sensor 137 utilizing processes as disclosed herein.

The housings shown in FIGS. 59 and 60 may be configured for axial insertion (e.g., insertion along the length of the proximal end portion of the elongate sensor 137) for coupling with other structures. In examples, the sealing members such as o-rings (or injection molded soft elastomers) may provide a seal with the other structures upon axial insertion. The electrical contacts 858a, b may electrically connect with external electrical contacts in manners disclosed herein. In examples, insertion transverse or perpendicular to the axis of the housings and the proximal end portion of the elongate sensor may be utilized.

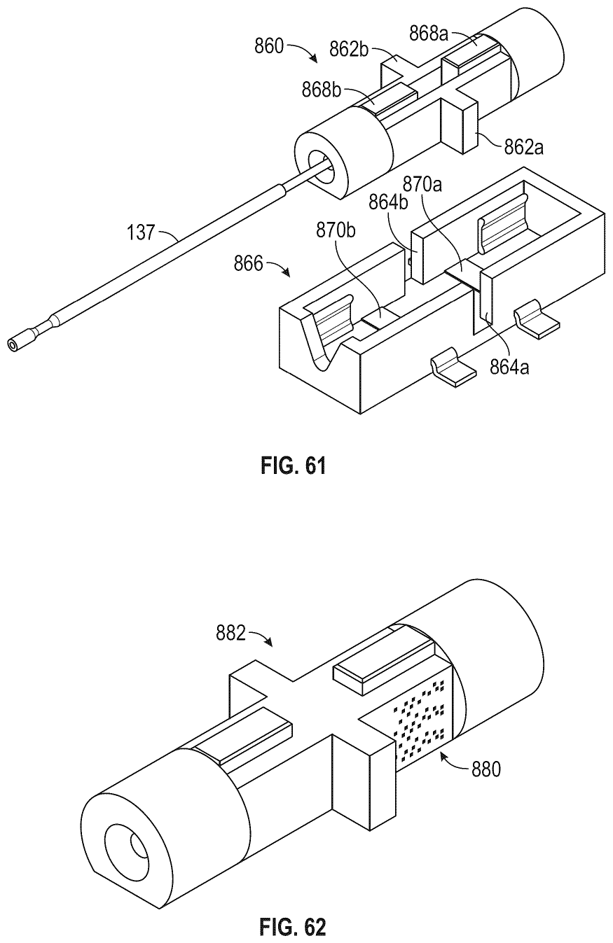

FIG. 61 illustrates a variation in which the overmolded housing 860 includes mating features 862a, b in the form of posts protruding from the outer surface of the housing 860. The posts may be received in mating features in the form of slots 864a, b of a socket 866.

The electrical contacts 868a, b may be configured for outer surface or surface-to-surface connection with respective electrical contacts 870a, b of the socket 866.

In examples, an identifier may be positioned on a portion of a housing. FIG. 62 illustrates an example in which an identifier 880 comprises a code that is positioned on a portion of a housing 882. The identifier 880 may be utilized for tracking the analyte sensor system or module during manufacture, testing, and/or assembly. The identifier 880 may be utilized to track characteristics of the housing 882, including calibration data for the housing 882. Any of the analyte sensor systems disclosed herein may utilize an identifier in examples.

FIG. 63 illustrates a variation in which an elongate sensor in the form of a planar sensor (e.g., flat sensor 403) is utilized with the overmolded body 402. An exploded view of the analyte sensor module is shown in FIG. 63. The body 402 may be overmolded upon a connection portion 405 of the planar sensor that is positioned at a proximal end portion 407 of the elongate sensor. The connection portion 405 includes a planar substrate. A resulting configuration is illustrated in FIG. 67 (yet with the connection portion 900 in FIG. 67 including one or more openings).

The features of any of the examples of FIGS. 1-63 may be utilized solely or in combination with any other example herein.

FIG. 64 illustrates a variation in a configuration of an elongate sensor 902 that may be utilized in examples herein. The elongate sensor 902 may include a distal end portion 904 and a proximal end portion 906. The distal end portion 904 may be configured to be inserted into skin of a host (an in vivo portion) and the proximal end portion 906 may be configured to be positioned exterior of the skin of the host (an ex vivo portion). The elongate sensor 902 may operate in a similar manner as discussed regarding the elongate sensor 137 (for example as discussed in regard to FIGS. 1-3D). The elongate sensor 902, however, may comprise a planar sensor. The planar sensor may include a planar substrate that components of the planar sensor are positioned upon.

For example, the distal end portion 904 of the elongate sensor 902 includes an insertion portion 908. The insertion portion 908 is configured for insertion into the skin of a host. The insertion portion 908 may include a planar substrate that components of the insertion portion 908 are positioned upon. The insertion portion 908 may include one or more electrodes (exemplary electrodes 910a, b are marked in FIG. 64) for detecting an analyte in a similar manner as discussed regarding the elongate sensor 137. The electrodes 910a, b are positioned on an elongated strip 912 of the planar substrate (such as a thin, flat, polymer flex circuit). Circuit traces 914a, b may extend along the length of the elongated strip 912 proximally to respective sensor electrical contacts 916a, b positioned at the proximal end portion 906 of the elongate sensor 902. The electrodes 910a, b are positioned on the elongated strip 912 to be disposed within the skin of the host for detecting the analyte therein.

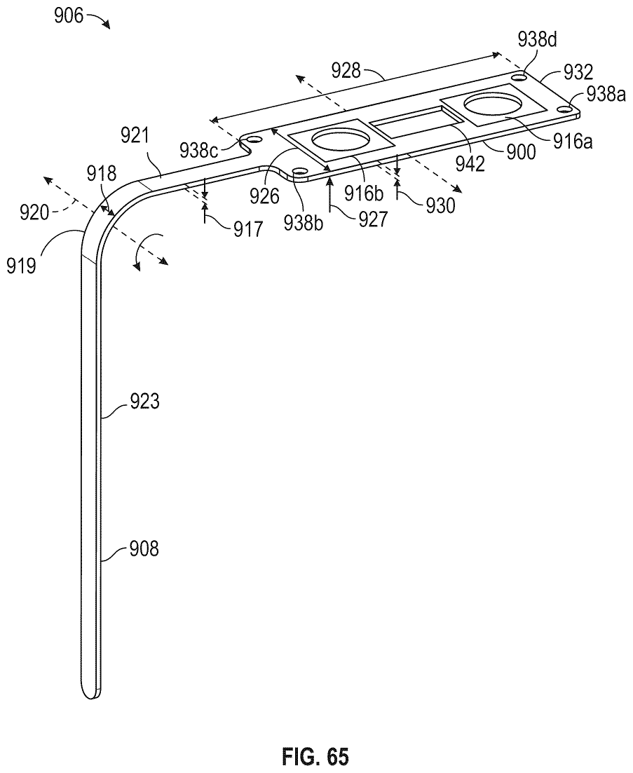

In examples, the insertion portion 908 is planar and has a thickness 917 (marked in FIG. 65) and a width 918 that is greater than the thickness 917. The insertion portion 908 is configured to bend (marked with the curved arrow in FIG. 65) about an axis 920 (marked in FIG. 65) that extends in the dimension that the width 918 extends along. The bend 919 may be positioned between a proximal section 921 of the insertion portion 908 and a distal section 923 of the insertion portion 908. The insertion portion 908 is shown to extend in a downward direction in FIG. 65, yet the direction of bend may alternatively be in an upward direction in examples (or a side direction as desired). The distal section 923 may extend distally towards the skin of the host. The insertion portion 908 may be sufficiently flexible to allow for a bend of the insertion portion 908, yet sufficiently rigid for insertion into the skin of the host. The elongate sensor 902 may be a bent elongate sensor.

The proximal end portion 906 of the elongate sensor 902 includes a connection portion 900 having the sensor electrical contacts 916a, b positioned thereon. The connection portion 900 may include a planar substrate that components of the connection portion 900 are positioned upon. The connection portion 900 may comprise an electrical connection portion of the elongate sensor 902 for electrical connection with other electrical contacts (e.g., for transmitting an electrical signal corresponding to the detected analyte as disclosed herein). In examples, the sensor electrical contacts 916a, b may be spaced from each other in a longitudinal direction along a length of the connection portion 900 as shown in FIG. 64. In examples, other configurations (e.g., spaced from each other in a lateral direction, or a combination of longitudinal and lateral spacing, among other configurations) may be utilized. The sensor electrical contacts 916a, b may be aligned with each other in the longitudinal direction as shown in FIG. 64.

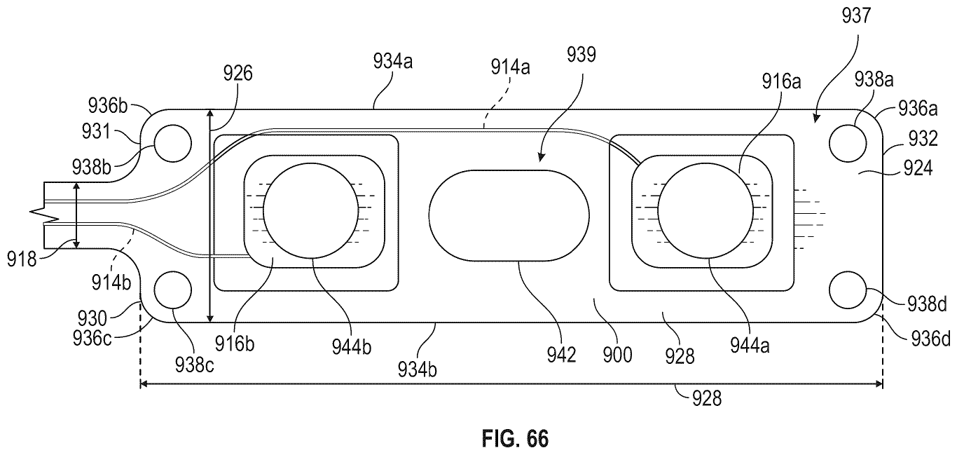

In examples, the sensor electrical contacts 916a, b may be positioned on an upper surface 922 and on a lower surface 924 (marked in FIG. 66) of the planar substrate of the connection portion 900 that faces opposite the upper surface 922. The electrical contacts 916a, b, for example, may respectively include exposed electrical contact surfaces on both the upper surface 922 and the lower surface 924 of the connection portion 900 (and electrically connected with the respective circuit traces 914a, b). In examples, the sensor electrical contacts 916a, b may be positioned on only an upper surface 922 or a lower surface 924 of the connection portion 900, among other configurations.

The connection portion 900 may be planar and may have a width 926 and a length 928, with the length 928 being greater than the width 926. The width 926 of the connection portion 900 may extend in the width dimension of the insertion portion 908. As such, the connection portion 900 may extend in the dimension that the width 918 of the insertion portion 908 extends along. The connection portion 900 may extend in a plane that is parallel or coextensive with the plane of the insertion portion 908 when the insertion portion 908 is flattened (or with the plane of the proximal section 921 of the insertion portion 908). The connection portion 900 may be configured to receive one or more electrical contacts (such as electrical contacts 518a, b shown in FIG. 68, or electrical contacts 960a, b shown in FIG. 70) in a direction 927 (marked in FIG. 65) that is transverse (e.g., perpendicular) to the dimension that the connection portion 900 extends in.

The connection portion 900 may have a thickness 930 (marked in FIG. 65) that may be the same as the thickness 917 of the insertion portion 908. The same thickness may result from the elongate sensor 902 being formed from a unitary sheet of materials having uniform thickness, which may be stamped, die cut, or laser cut (among other forms of separation) to cut out the shape of the elongate sensor 902. The insertion portion 908 may then be bent as shown in FIG. 65 in a desired direction. In examples, the connection portion 900 may have a different thickness (e.g., greater or lesser thickness) than the insertion portion 908. Other configurations may be utilized in examples.

The width 926 of the connection portion 900 may be greater than the width 918 of the insertion portion 908 as shown in FIG. 66 for example. As such, the connection portion 900 may include a distal outer edge 931 formed by the greater width 926 of the connection portion 900. In examples, the width 926 of the connection portion 900 may be the same as, or lesser than the width 918 of the insertion portion 908.

The connection portion 900 may include the distal outer edge 931, a proximal outer edge 932, and two side outer edges 934a, b. The distal outer edge 931 and proximal outer edge 932 may extend laterally and may extend substantially parallel with each other. The two side outer edges 934a, b may be longitudinal edges that extend along the length of the elongate sensor 902 and that extend substantially parallel with each other (and transverse to the distal and proximal outer edges 931, 932). The connection portion 900 may have a rectangular shape as shown in FIG. 66, although other configurations may be utilized in examples.

The outer edges 931, 932, 934a, b may meet at respective corners 936a, b, c, d of the connection portion 900.

In examples, the connection portion 900 may include one or more openings. In FIG. 66 for example, the connection portion 900 includes a variety of forms of openings having different purposes or functions. In examples, one or more of the openings may be utilized as desired, which may have a same purpose or function or a different purpose or function as desired.

The connection portion 900 may include one or more openings 938a, b, c, d that are configured for receiving material of the body 940 (marked in FIG. 67) for securing the connection portion 900 to the body 940. The openings 938a, b, c, d may extend through the connection portion 900 from the upper surface 922 to the lower surface 924. The material of the body 940 may insert into the respective openings 938a, b, c, d fully or partially to secure to the connection portion 900. The material of the body 940 may insert into the openings 938a, b, c, d during a molding process of the body 940 (e.g., when the body 940 is molded onto the connection portion 900).

The openings 938a, b, c, d may be positioned in a variety of locations on the connection portion 900 as desired. For example, the openings 938a, b, c, d may be positioned at the respective corners 936a, b, c, d of the connection portion 900 in examples. The openings 938a, b, c, d may be positioned at an outer peripheral region 937 of the connection portion 900. Other locations for the one or more openings 938a, b, c, d may be utilized in examples. The outer peripheral region 937 of the connection portion 900 (and the openings 938a, b, c, d) may be covered by the body 940 during the molding process, as shown in FIG. 67 for example. The material of the body 940 entering the openings 938a, b, c, d during the molding process may secure the connection portion 900 to the body 940. A central region 939 of the connection portion 900 may remain exposed following the molding process of the body 940. The central region 939 may include the sensor electrical contacts 916a, b.

The connection portion 900 may include one or more openings 942 for a sealing member or fill material to pass through. The connection portion 900 is shown having one such opening 942 in FIG. 66, yet in examples multiple of such openings may positioned on the connection portion 900 as desired. The opening 942 may be positioned at the central region 939 of the connection portion 900 in examples (as shown in FIG. 66). The opening 942 may be positioned between the sensor electrical contacts 916a, b. In examples, other positions for the one or more openings 942 may be utilized as desired.

The opening 942 may extend through the connection portion 900 from the upper surface 922 to the lower surface 924. As such, the opening 942 serves as a flow channel for any sealing member or fill material provided on one side of the connection portion 900 to flow through to the other side of the connection portion 900, whether during a molding process or dispensing of a liquid cured material (e.g., a liquid cured encapsulant).

The sensor electrical contacts 916a, b may include one or more openings 944a, b. The one or more openings 944a, b may be configured to receive an electrical contact (e.g., electrical contacts 518a, b shown in FIG. 68, or electrical contacts 960a, b shown in FIG. 70, or another form of electrical contact) for electrical connection with such electrical contact. The one or more openings 944a, b may pass through a respective one of the sensor electrical contacts 916a, b, and may extend through the connection portion 900 from the upper surface 922 to the lower surface 924. The exposed electrical contact surfaces on both the upper surface 922 and the lower surface 924 of the connection portion 900 of the sensor electrical contacts 916a, b may surround or encircle the one or more openings 944a, b. In examples, other configurations may be utilized.

The one or more openings 944a, b may allow the material of the electrical contact (e.g., electrical contacts 518a, b shown in FIG. 68, or electrical contacts 960a, b shown in FIG. 70, or another form of electrical contact) to pass therethrough, to be positioned on both the upper surface 922 and the lower surface 924 of the connection portion 900 (as represented in FIGS. 68 and 70). As such, increased contact surface areas for electrical connection and improved electrical connection with both sides of the sensor electrical contacts 916a, b (on the upper surface 922 and the lower surface 924 of the connection portion 900) may result. Improved mechanical securement of the electrical contacts to the sensor electrical contacts 916a, b and the connection portion 900 may additionally or alternatively result.

In examples, other configurations of openings may be utilized as desired.

FIG. 67 illustrates a top view of the body 940 having been molded upon at least a portion of the elongate sensor 902. The body 940 is molded upon the proximal end portion 906 of the elongate sensor 902. The body 940 is configured similarly as the body 402 (shown in FIG. 4) and includes the features of the body 402 unless stated otherwise. The body 940 comprises a housing and a frame 943 for the elongate sensor 902.

The body 940 molds onto and covers the outer peripheral region 937 of the connection portion 900 and onto the proximal section 921 of the insertion portion 908 (proximal of the position of the bend 919). The body 940 forms a seal upon the outer surface of the elongate sensor 902 that the body 940 is molded onto. The body 940 may expose the sensor electrical contacts 916a, b in the interior chamber 950 of the body 940. The body 940, for example, may include one or more openings 952, 954 that may allow for electrical connection of the sensor electrical contacts 916a, b of the elongate sensor 902 with electrical contacts of a sensor electronics module.

The body 940 may mold onto the elongate sensor 902 such that the upper surface 922 of the connection portion 900 faces towards an upper opening 952 of the body 940, and the lower surface 924 of the connection portion 900 faces towards the lower opening 954 (marked in FIG. 68). The connection portion 900 extends transverse or perpendicular to the direction of insertion of external electrical contacts towards the connection portion 900. The connection portion 900 may extend parallel to the planes of the openings 952, 954 and may extend parallel to a surface of a skin for receiving the elongate sensor 902 in examples. Other orientations may be utilized in examples.

The body 940 and elongate sensor 902 may comprise an analyte sensor system or sensor module 941 for coupling with a receiving portion of an external structure such as a socket. The sensor module 941 may couple with the receiving portion in any of the variety of manners as disclosed herein. For example, referring to FIG. 68, in examples, the sensor module 941 may be coupled with an external structure such as a socket in a similar manner as disclosed in regard to FIGS. 8C-11. The body 940 may be inserted into such a receiving portion in the same manner as represented in FIG. 9.

The electrical contacts 518a, b may be coupled to the sensor electrical contacts 916a, b in a similar manner as disclosed in regard to FIGS. 8C-11. For example, the electrical contacts 518a, b may comprise a cured liquid or cured conductive liquid (e.g., a cured adhesive, gel, epoxy, etc.) that may be conductive and may allow for electrical connection with the sensor electrical contacts 916a, b of the elongate sensor 902. The liquid of the electrical contacts 518a, b may pass through the respective openings 944a, b to be positioned on both sides of the sensor electrical contacts 916a, b (on the upper surface 922 and the lower surface 924 of the connection portion 900) prior to solidification.

The conductive liquid solidifies to form a solidified conductive liquid. In examples, other forms of electrical contacts (e.g., pads or pucks, protrusions such as pins, electrically conductive film, magnetic materials, magnetic conductive adhesive, uv curable materials such as uv curable die attach, among others) may be utilized as desired. The form of electrical contacts 518a, b is not limited to wet processes.

In examples, and similar to the process disclosed in regard to FIGS. 8C-11, a sealing member 528 may be provided to seal the electrical connection between the electrical contacts 518a, b and the electrical contacts 530a, b of the circuit board substrate 524. Further sealing may be provided between the sensor electrical contacts 916a, b of the elongate sensor 902 themselves, or any other of the contacts, which may be for purposes of electrical isolation between the contacts.

In examples, a fill material, such as a liquid dispensed material may comprise the sealing member 528. The liquid dispensed material, similar to a configuration as shown in FIG. 11, may be dispensed into the socket 519 and may fill areas around the respective contacts 518a, b, 530a, b. The liquid dispensed material may fill the interior chamber 950. The liquid dispensed material may pass through the opening 942 of the connection portion 900 to be positioned on both sides of the connection portion 900 (on the upper surface 922 and the lower surface 924 of the connection portion 900) prior to solidification. The liquid dispensed material may be cured to seal the socket 519 from moisture ingress, which may otherwise undesirably produce an electrical short or other errors (e.g., signal errors) in the system.

In examples, other forms of sealant (e.g., elastomeric bodies) may be utilized as desired. Other forms of sensor modules may be utilized in examples.

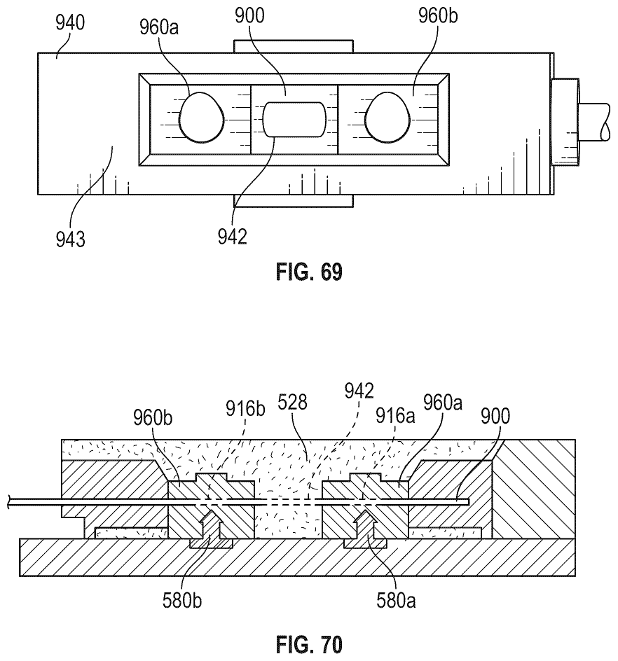

FIG. 69, for example, illustrates a variation of the sensor module 941 in which the sensor module includes one or more molded electrical contacts 960a, b therein. The electrical contacts 960a, b may be configured similarly as the electrical contacts 552a, b and include the features of the electrical contacts 552a, b (marked in FIG. 13) unless stated otherwise. The electrical contacts 960a, b may be formed in a molding process in a similar manner as disclosed regarding the electrical contacts 552a, b and may be made of the same materials as disclosed regarding the electrical contacts 552a, b. The electrical contacts 960a, b may be retained within the frame 943 of the body 940.

The electrical contacts 960a, b may be positioned at the respective sensor electrical contacts 916a, b (as marked in the cross sectional view of FIG. 70). The electrical contacts 960a, b may pass through the respective openings 944a, b (marked in FIG. 66) of the sensor electrical contacts 916a, b during the molding process, to be positioned on both sides of the sensor electrical contacts 916a, b (on the upper surface 922 and the lower surface 924 of the connection portion 900). In examples, the use of the openings 944a, b may be excluded, and the electrical contacts 960a, b may be molded onto only one side of the connection portion 900 to contact the sensor electrical contacts 916a, b (e.g., the side of the connection portion 900 that will electrically connect with sensor electronics).

The electrical contacts 960a, b may protrude from the lower surface of the body 940, or may be positioned at the height of the lower surface, or at a lesser height in examples. The electrical contacts 960a, b may be configured to electrically connect with external electrical contacts in a similar manner as disclosed regarding FIGS. 17-33. For example, a surface-to-surface connection may be provided (as represented in FIG. 17), or in examples the electrical contacts 960a, b may be penetrated by respective conductive protrusions such as pins 580a, b (as represented in FIG. 27 and as marked in FIG. 70). Any other form of conductive protrusion disclosed herein may be utilized. In examples, other forms of electrical connection as disclosed herein may be utilized.

In examples, a sealing member 528 may be utilized in a similar manner as disclosed in regard to FIG. 68. The sealing member 528 may flow through the opening 942 in a liquid state. Other forms of sealing may be utilized in examples. For example, a sealing member 652 or a fill material 660 as represented in FIG. 29 may be utilized in examples. In examples, a sealing member 692 as represented in FIG. 35 that covers the lower surfaces of the electrical contacts 960a, b may be utilized. The sealing members or fill materials may flow through the opening 942 during a molding process. A resulting configuration may include the features as disclosed regarding FIGS. 34-36, yet with a configuration of a connection portion and insertion portion as disclosed herein. Various combinations of features across examples may be utilized as desired.

In examples, the analyte sensor systems or sensor modules disclosed in regard to FIGS. 63-70 may be utilized with any form of receiving portion or socket as disclosed herein, including any receiving portion or socket disclosed in regard to FIGS. 37-58, among other forms of receiving portions or sockets. The analyte sensor systems or sensor modules disclosed in regard to FIGS. 63-70 may be utilized in any position or orientation disclosed herein, including any receiving position or orientation disclosed in regard to FIGS. 37-58, among other positions or orientations.

The features of any of the examples of FIGS. 63-70 may be utilized solely or in combination with any other example herein.

Other forms of analyte sensor systems or modules may be utilized in examples herein. FIGS. 71-73 illustrate a variation of a configuration of the analyte sensor system or module represented in FIGS. 28-33. The analyte sensor system or sensor module 970 may include the features of the analyte sensor system or module represented in FIGS. 28-33 unless stated otherwise.

The sensor module 970 may include the elongate sensor 137, and may include a body 972 molded upon at least a portion of the elongate sensor 137. The body 972 may comprise a housing or elongate sensor housing that houses the proximal end portion 142 (marked in FIG. 74) of the elongate sensor 137. The housing may include multiple components in examples, each molded upon the elongate sensor 137 in a similar manner as disclosed with the features of the analyte sensor system or module represented in FIGS. 28-33. For example, the housing may include a frame 974, one or more electrical contacts 975a, b, and at least one sealing member 979. The frame 974 may include the features of the frame 554 shown in FIG. 28 unless stated otherwise. The one or more electrical contacts 975a, b may include the features of the electrical contacts 552a, b shown in FIG. 29 unless stated otherwise. The sealing member 979 may include the features of the sealing member 652 shown in FIG. 29 unless stated otherwise.

The frame 974 is shown in isolation in FIGS. 74 and 75 molded upon the proximal end portion 142 of the elongate sensor 137. The frame 974 may be molded upon the elongate sensor 137 in a similar manner as other forms of frames disclosed herein. The frame 974 may be molded upon the elongate sensor 137 to form a seal upon the outer surface of the elongate sensor 137.

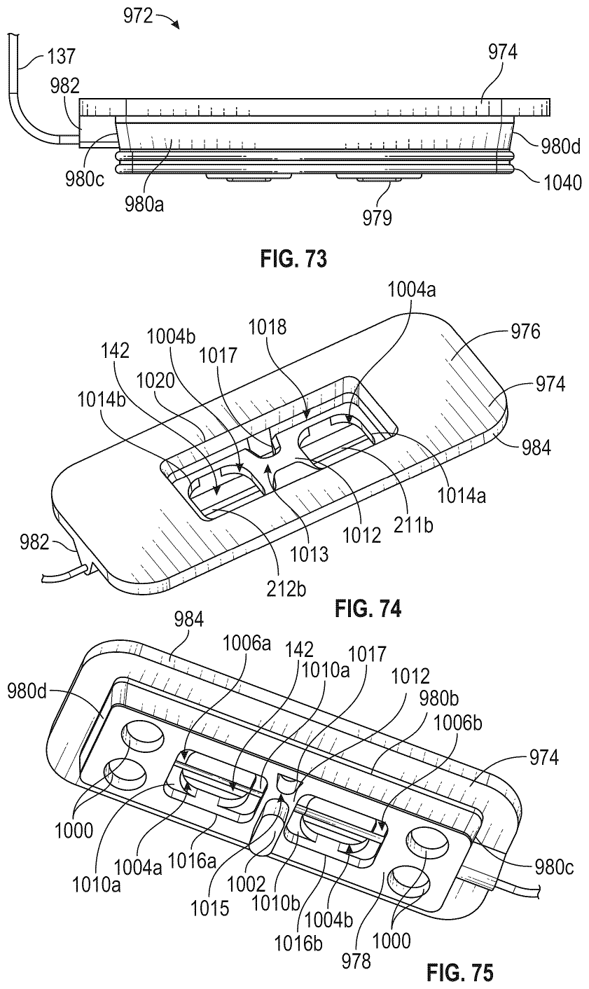

The frame 974 may include an upper outer surface 976, a lower outer surface 978 (marked in FIG. 75) and one or more outer side surfaces 980a-d (marked in FIGS. 73 and 75). The surfaces of the frame 974 may include the features of the corresponding surfaces of the body 402 shown in FIG. 4 unless stated otherwise.

The upper outer surface 976 may comprise a flat or planar surface in examples. The upper outer surface 976 may form the upper outer surface of the body 972 or housing in examples. The outer side surfaces 980a-d may comprise angled surfaces that angle radially inward in a direction of the frame 974 from the upper outer surface 976 towards the lower outer surface 978. The angle or taper of the outer side surfaces 980a-d may allow for improved mating with a receiving portion of an external structure such as a socket. For example, the external structure may have a corresponding angle or taper that may allow for improved alignment and mating between the body 972 and the external structure. In examples, the outer side surface 980c may include a mating feature 982 that includes the features of the mating feature 432 of the body 402 shown in FIG. 4.

The body 972 includes a flange 984 that protrudes radially outward from the body 972 and extends circumferentially about the body 972. The flange 984 may have a planar or flat shape, forming a ring about the body 972. The flange 984 is a part of the frame 974 and forms the upper outer surface 976 of the frame 974. The flange 984 protrudes radially outward from the outer side surfaces 980a-d of the frame 974. The flange 984 is configured to allow for improved mating with a receiving portion of an external structure such as a socket. For example, referring to FIG. 81, a corresponding receiving portion or socket 990 may include a recess 992 that is shaped to receive the flange 984. The flange 984 may fit within the recess 992 upon insertion of the sensor module 970 into the socket 990. The upper outer surface 976 may extend flush with the bottom outer surface 994 of the wearable housing 996 in examples (although in examples the upper outer surface 976 may be raised or lowered from the surface 994 as desired).

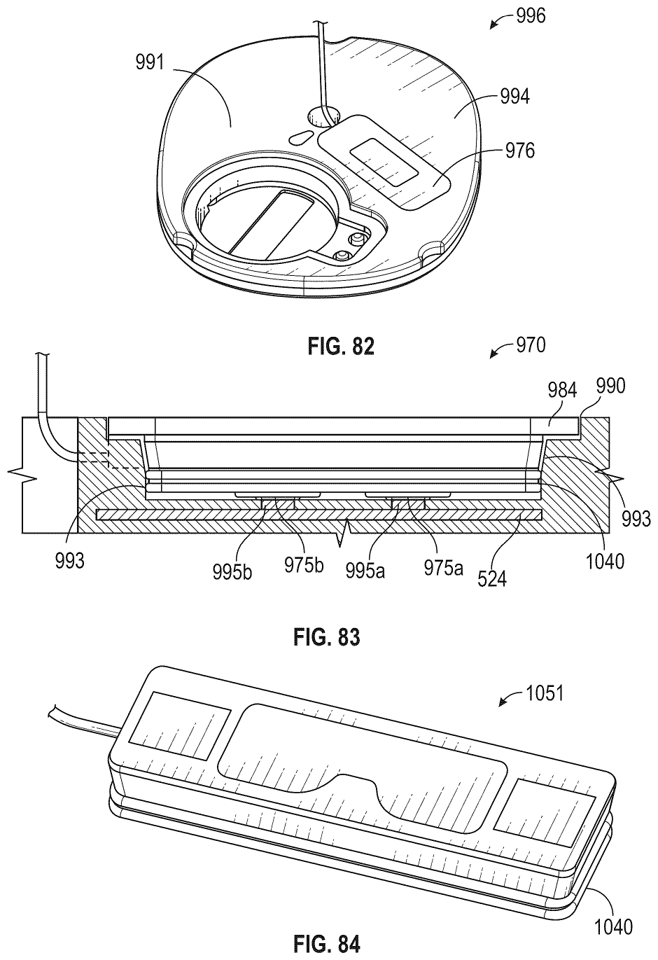

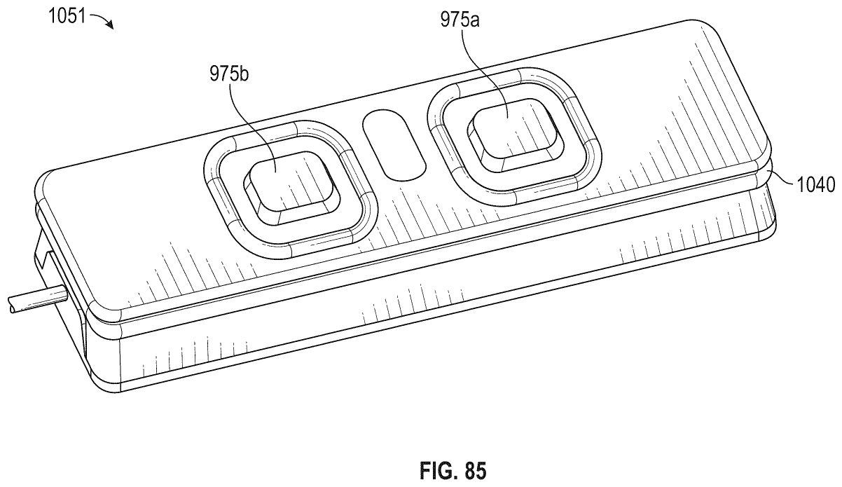

In examples, the flange 984 may be configured for bonding with at least a portion of the socket 990 configured to receive the body 972. For example, in a configuration as shown in FIG. 82, the outer edge of the flange 984 may be welded (e.g. laser welded or ultrasonically welded), adhered, or otherwise bonded to the adjacent portion of the socket 990 (e.g., the bottom outer surface 994 of the wearable housing 996) for further mechanical connection with the socket 990. Additionally or alternatively, improved sealing with the socket 990 may result. In examples (as shown in FIGS. 84 and 85), the flange 984 may be excluded.

Referring to FIG. 75, the lower outer surface 978 may face opposite the upper outer surface 976. The lower outer surface 978 may include one or more securing features 1000, 1002 that may be utilized for securing the sealing member 979 or other forms of sealing members or fill material to the frame 974. The securing features 1000, for example, may comprise recesses or cavities configured to receive the material forming the sealing member 979. The securing feature 1002 may comprise a protrusion for extending through the material forming the sealing member 979. As such, a reduced possibility of twisting or other forms of release or separation from the frame 974 may result. Other configurations may be utilized in examples.

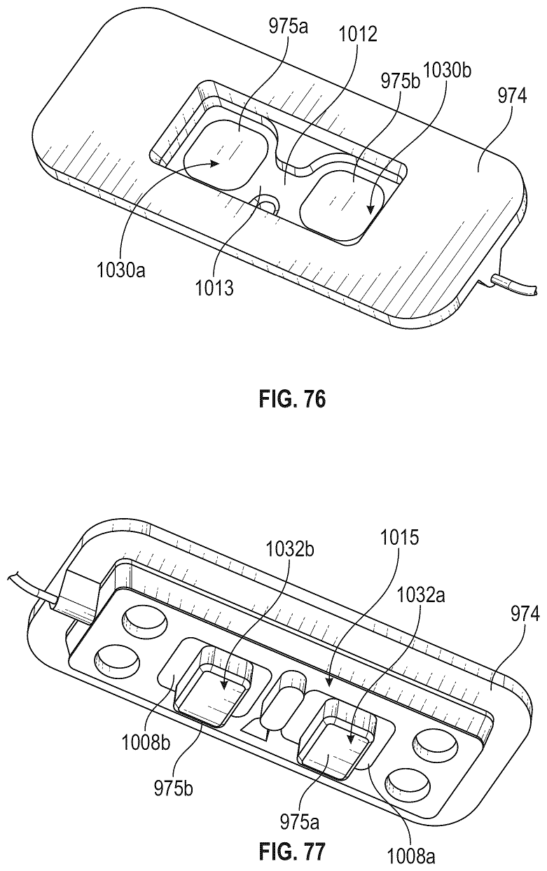

The frame 974 may include a first interior chamber 1004a and a second interior chamber 1004b in examples. The interior chambers 1004a, b may be configured to receive a respective one of the sensor electrical contacts 211b, 212b and the electrical contacts 975a, b marked in FIG. 76 (with the first electrical contact 975a positioned within the first interior chamber 1004a, and the second electrical contact 975b positioned within the second interior chamber 1004b).

The first interior chamber 1004a may be spaced from the second interior chamber 1004b in a longitudinal direction along a length of the proximal end portion 142 of the elongate sensor 137. As such, the interior chambers 1004a, b are positioned to receive electrical contacts 975a, b corresponding to the position of the respective sensor electrical contacts 211b, 212b (marked in FIG. 74) (with the sensor electrical contacts 211b, 212b positioned within the interior chambers 1004a, b).

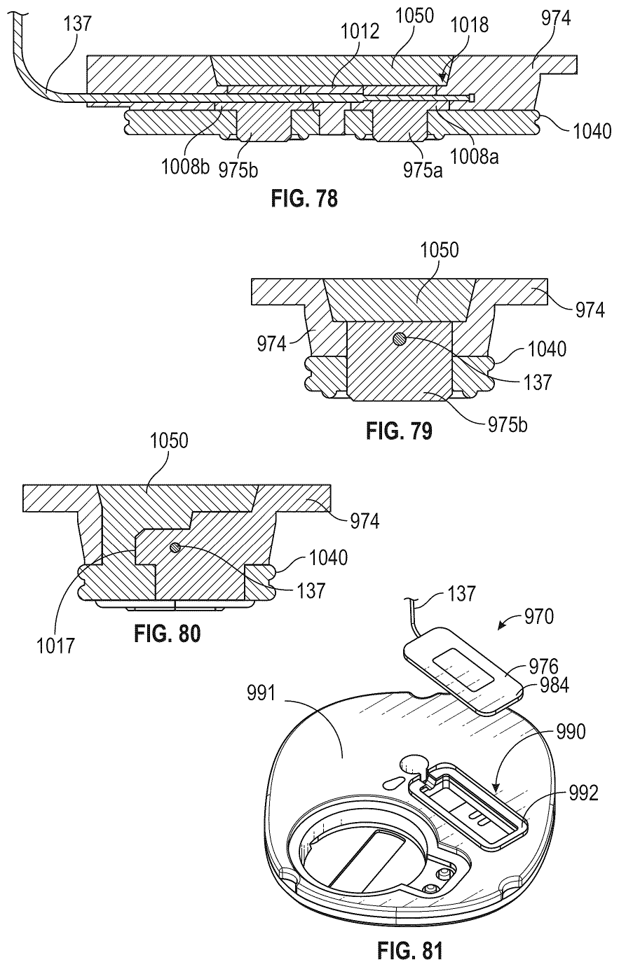

Referring to FIG. 75, the interior chambers 1004a, b may include respective recesses or cavities 1006a, b configured to receive flanges 1008a, b (marked in FIG. 78) of the respective electrical contacts 975a, b. The recesses or cavities 1006a, b may include supporting surfaces 1010a, b for supporting the flanges 1008a, b of the respective electrical contacts 975a, b. As such, the supporting surfaces 1010a, b may resist a force applied to the electrical contacts 975a, b in a direction towards the upper outer surface 976 of the body 972 (e.g., a force opposite a direction of insertion of the body 972 into a socket). In examples, other configurations may be utilized.

The frame 974 may include a divider 1012 that may divide the first interior chamber 1004a from the second interior chamber 1004b. The divider 1012 may extend laterally, lateral relative to the length of the proximal end portion 142 of the elongate sensor 137. The divider 1012 may serve to provide electrical and mechanical isolation between the electrical contacts 975a, b (and the sensor electrical contacts 211b, 212b). The divider 1012 may include an upper surface 1013 and a lower surface 1015 (marked in FIG. 75). In examples, the proximal end portion 142 of the elongate sensor 137 may extend through the divider 1012 (the divider 1012 may be molded onto the proximal end portion 142 of the elongate sensor 137 between the sensor electrical contacts 211b, 212b).

In examples, the frame 974 may include an opening 1017 forming a flow channel for material to pass from the upper surface 1013 to the lower surface 1015. The material may comprise material forming the sealing member 979 or other forms of sealing members or fill material for the frame 974. The flow channel may allow for flow of material between the upper surface 1013 and the lower surface 1015 during a molding process.

In examples, the first interior chamber 1004a and the second interior chamber 1004b may include respective upper openings 1014a, 1014b and lower openings 1016a, 1016b. The upper openings 1014a, 1014b may be positioned on the upper surface 1013 and the lower openings 1016a, 1016b may be positioned on the lower surface 1015. The lower openings 1016a, 1016b may allow for electrical connection of the sensor electrical contacts 211b, 212b with electrical contacts of a sensor electronics module.

The first interior chamber 1004a and second interior chamber 1004b may each comprise sub-chambers of an interior chamber 1018 or main interior chamber (marked in FIG. 74) of the frame 974. The main interior chamber 1018 may extend from the upper openings 1014a, 1014b of the interior chambers 1004a, b to a main upper opening 1020 of the frame 974. The main upper opening 1020 may be positioned on the upper outer surface 976 of the frame 974. The first interior chamber 1004a and second interior chamber 1004b may be continuous with the main interior chamber 1018.

FIGS. 76 and 77 illustrate the one or more electrical contacts 975a, b within the frame 974 and having been molded to the proximal end portion 142 of the elongate sensor 137. The one or more electrical contacts 975a, b may surround the sensor electrical contacts 211b, 212b. As discussed regarding the analyte sensor system or module represented in FIGS. 28-33, the electrical contacts 975a, b may either be molded initially, with the frame 974 molded thereafter, or the frame 974 may be molded initially, with the electrical contacts 975a, b molded into the frame 974. The body 972 may comprise a multi-shot overmolding upon the elongate sensor 137. The electrical contacts 975a, b are shown to be molded with respective upper outer surfaces 1030a, b, lower outer surfaces 1032a, b (marked in FIG. 77), and respective outer side surfaces including flanges 1008a, b. The electrical contacts 975a, b may be made of a similar material as other forms of electrical contacts disclosed herein and may be molded in a similar manner.

Referring to FIG. 76, the respective upper outer surfaces 1030a, b of the electrical contacts 975a, b may be flush with the upper surface 1013 in examples. Referring to FIG. 77, the lower outer surfaces 1032a, b of the electrical contacts 975a, b may protrude from the lower outer surface 978 of the frame 974 in examples. Other configurations may be utilized as desired.

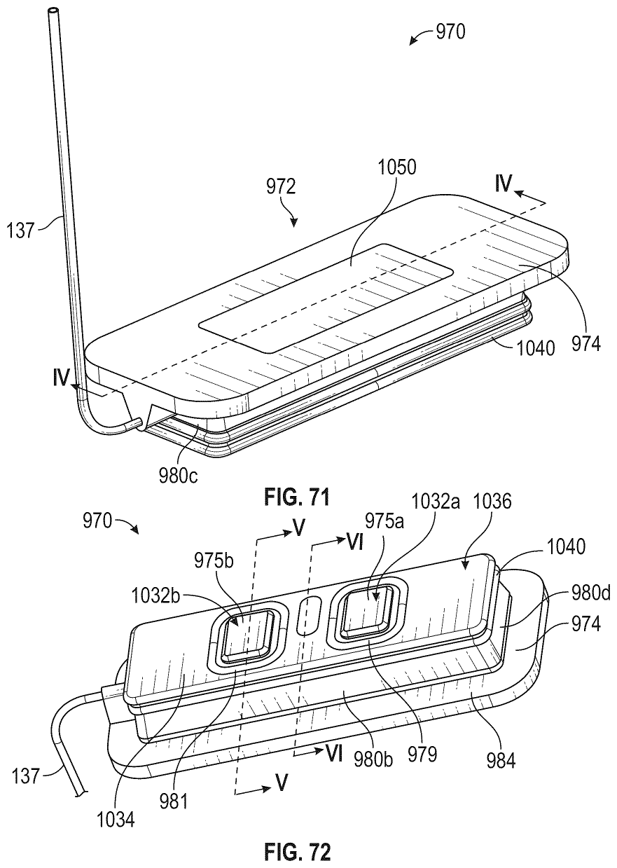

In a configuration as shown in FIGS. 76 and 77, the sealing member or fill material may be molded to produce a resulting configuration shown in FIGS. 71-73. The sealing member 979 shown in FIG. 72 is formed in such a molding process. The sealing member 979 may be configured similarly as the sealing member 652 represented in FIG. 29, forming one or more ribs and/or grooves for forming a seal with a surface. The ribs and/or grooves may extend around the lower outer surface 1032a of the electrical contact 975a. A similar sealing member 981 may be formed to extend around the lower outer surface 1032b of the electrical contact 975b.

Similar to the configuration of the sealing member 652 represented in FIG. 29, the sealing members 979, 981 may be part of other forms of sealing members and/or fill material that may be molded. The sealing member may be made of a similar material as other forms of sealing members disclosed herein and may be molded in a similar manner. A similar molding process as with the sealing member 652 may be utilized. For example, the material forming the sealing members 979, 981 may form a sealing member 1034 comprising the bottom layer of the body 972. The sealing member 1034 may form an outer surface of the body 972 (the lower outer surface 1036 of the body 972). The sealing member 1034 may cover the lower outer surface 978 of the frame 974 (marked in FIG. 75).

The lower outer surfaces 1032a, b of the respective electrical contacts 975a, b may protrude from the lower outer surface 1036 of the body 972 as shown in FIG. 72. Such a feature may allow for surface-to-surface connection with external electrical contacts in a manner as disclosed herein. The sealing member 1034 (and sealing members 979, 981) may seal such a connection (e.g., a face seal). In examples, the height of the lower outer surfaces 1032a, b of the respective electrical contacts 975a, b may be reduced such that the sealing member 1034 entirely covers the lower outer surfaces 1032a, b of the respective electrical contacts 975a, b (as represented in FIGS. 34-36 for example). In such a configuration the sealing member 1034 may be penetrated by electrical contacts as disclosed herein for electrical connection (e.g., penetrated with conductive protrusions). Other configurations may be utilized as desired.

In examples, the body 972 includes a sealing member 1040 (marked in FIG. 71) configured to form a radial seal. The sealing member 1040 may be configured to form the radial seal with a receiving portion of an external structure such as a socket that is configured to receive the body 972. The sealing member 1040 may include one or more ribs or grooves extending circumferentially about the outer surface (e.g., outer side surface) of the body 972. As shown in the side view of FIG. 73, the sealing member 1040 may protrude from the outer side surfaces 980a-d of the frame 974 such that the sealing member 1040 may contact side surfaces of a socket or other form of receiving portion of an external structure to seal with such surfaces.

The sealing member 1040 may comprise a double rib or dual rib structure as shown in FIG. 71. Other configurations may be utilized as desired.

In examples, the sealing member 1040 may be integral with the sealing member 1034 (and sealing members 979, 981). In examples, the sealing member 1040 may be formed separately from the sealing member 1034, or the body 972 may lack one or more of the sealing members 1040, 1034. In examples, the sealing member 1040 may be formed during a molding process for the sealing member 1034. For example, the sealing members may be formed as a part of fill material that fills the main interior chamber 1018 as disclosed herein. The fill material may pass through the opening 1017 to pass between the upper surface 1013 and the lower surface 1015. The sealing members may be formed in such a molding process.

An upper portion 1050 (marked in FIG. 71) of the fill material may fill the main interior chamber 1018. The lower portion of the fill material positioned on an opposite side of the divider 1012 may form the sealing members 979, 981, 1034, 1040.

FIG. 78, for example, illustrates a cross sectional view along line IV-IV in FIG. 71. The fill material fills the main interior chamber 1018 in the molding process to form the sealing members 979, 981, 1034, 1040 on the opposite side of the divider 1012.

FIG. 79 illustrates a cross sectional view along line V-V in FIG. 72. FIG. 80 illustrates a cross sectional view along line VI-VI in FIG. 72. The fill material is shown to extend through the opening 1017.

Various other configurations may be utilized as desired.

FIGS. 81-83 illustrate a coupling between the sensor module 970 and a receiving portion or socket 990. The socket 990 may comprise any form of socket disclosed herein. For example, FIG. 81 illustrates the socket 990 as a recess on a bottom outer surface 991 of a wearable housing (similar to a configuration as shown in FIG. 25 or 37). The socket 990, however, may include the recess 992 for receiving the flange 984 in examples. In examples, the recess 992 may be excluded from use.

The sensor module 970 is shown in FIG. 81 spaced from the socket 990. The sensor module 970 may be pressed in a direction transverse or perpendicular to a direction of the proximal end portion 142 of the elongate sensor 137 towards the socket 990. The sensor module 970 may be pressed into the socket 990 as represented in FIG. 82.

FIG. 83 illustrates a partial cross sectional view of the sensor module 970 within the socket 990. The sealing member 1040 may contact side walls 993 or side surfaces of the socket 990 to form a radial seal with the side walls 993. In examples, the flange 984 may be welded, adhered, or otherwise bonded to the socket 990 as disclosed herein. In examples, such a feature may be excluded (the flange 984 may not be bonded) and the sealing member 1040 may form a mechanical connection with the socket 990 (e.g., a press fit or friction fit as desired). One or more of the sealing members may further form a moisture seal of the electrical connection between the electrical contacts 975a, b and the external electrical contacts 995a, b of the socket 990 (which may be positioned upon a circuit board substrate 524).

Other configurations may be utilized in examples.

FIGS. 84 and 85, for example, illustrate a variation in which the body 1051 includes the features of the body 972, yet excludes the use of the flange 984. In such a configuration the sealing member 1040 may form a mechanical connection with a socket 990 (e.g., a press fit or friction fit as desired). One or more of the sealing members may further form a moisture seal of the electrical connection between the electrical contacts 975a, b and the external electrical contacts of a socket 990.

In examples, the analyte sensor systems or sensor modules disclosed in regard to FIGS. 71-85 may be utilized with any form of receiving portion or socket as disclosed herein, including any receiving portion or socket disclosed in regard to FIGS. 37-58, among other forms of receiving portions or sockets. The analyte sensor systems or sensor modules disclosed in regard to FIGS. 71-85 may be utilized in any position or orientation disclosed herein, including any receiving position or orientation disclosed in regard to FIGS. 37-58, among other positions or orientations.

The features of any of the examples of FIGS. 71-85 may be utilized solely or in combination with any other example herein.

Other variations may be provided in examples.

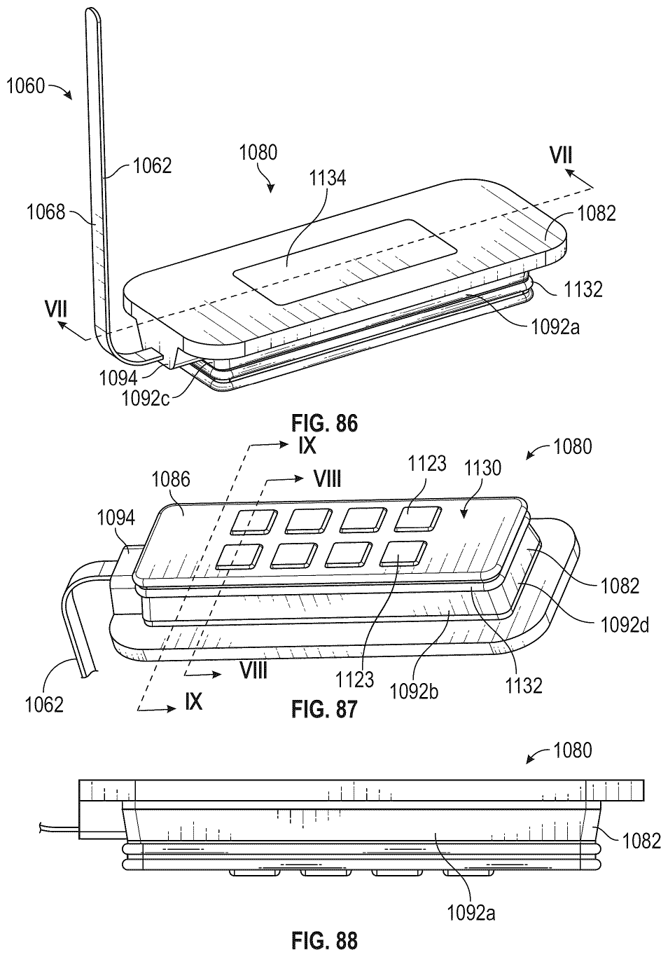

FIGS. 86-88 illustrate a variation of a configuration of the analyte sensor system or module 970 represented in FIGS. 71-83. The analyte sensor system or sensor module 1060 may include the features of the analyte sensor system or module 970 represented in FIGS. 71-83 unless stated otherwise.

The sensor module 1060 comprises a configuration of the sensor module 970 adapted for supporting an elongate sensor in the form of a planar sensor as represented in FIGS. 64-66 for example. The elongate sensor 1062 includes the features of the elongate sensor 902 as represented in FIGS. 64-66 unless stated otherwise.

FIGS. 89 and 90 illustrate an isolated view of the elongate sensor 1062. The elongate sensor 1062, similar to the elongate sensor 902, includes a distal end portion 1064 and a proximal end portion 1066. The distal end portion 1064 may be configured to be inserted into skin of a host (an in vivo portion) and the proximal end portion 1066 may be configured to be positioned exterior of the skin of the host (an ex vivo portion).

The distal end portion 1064 of the elongate sensor 1062 includes an insertion portion 1068. The insertion portion 1068 is configured for insertion into the skin of a host. The insertion portion 1068 may include a planar substrate and may include electrodes (similar to the exemplary electrodes 910a, b marked in FIG. 64) for detecting an analyte in a similar manner as discussed regarding the elongate sensor 137. The insertion portion 1068 may include electrodes, an elongated strip, and circuit traces configured similarly as the respective components of the insertion portion 908.

The elongate sensor 1062 may otherwise include the features of the elongate sensor 902. For example, the elongate sensor 1062 may include a connection portion 1070, sensor electrical contacts 1072, openings 1074, opening 1076, and openings 1078a-d that correspond to and include the features of the respective connection portion 900, sensor electrical contacts 916a, b, openings 938a-d, opening 942, and openings 944a, b of the elongate sensor 902.

The elongate sensor 1062, however, may include a greater number of sensor electrical contacts 1072 than the elongate sensor 902 represented in FIGS. 64-66. Exemplary sensor electrical contacts 1072a-d are marked in FIG. 89 (yet the elongate sensor 1062 is shown to include eight sensor electrical contacts 1072 in FIG. 89). The greater number of sensor electrical contacts 1072 may be due to the elongate sensor 1062 being configured to detect multiple different analytes (a multi-analyte sensor). In examples, the elongate sensor 1062 may be configured to detect a single analyte. A greater or lesser number of sensor electrical contacts may be utilized in examples as desired.

The sensor electrical contacts 1072 may be spaced from each other in a longitudinal direction along a length of the planar substrate of the connection portion 1070 in examples. For example, sensor electrical contacts 1072a and 1072c may be spaced from each other in the longitudinal direction. The sensor electrical contacts 1072a, c are aligned with each other in the longitudinal direction. Additionally or alternatively, sensor electrical contacts 1072 may be spaced from each other in a lateral direction that is lateral relative to a length of the connection portion 1070. For example, sensor electrical contacts 1072a and 1072b may be spaced from each other in the lateral direction. The sensor electrical contacts 1072a, b are aligned with each other in the lateral direction. Two longitudinal columns of sensor electrical contacts 1072 are shown in FIG. 89, with four lateral rows of sensor electrical contacts 1072. Each sensor electrical contact 1072 may include a respective electrical connection to a circuit trace and an electrode on the insertion portion 1068 as desired. Other configurations may be utilized in examples.

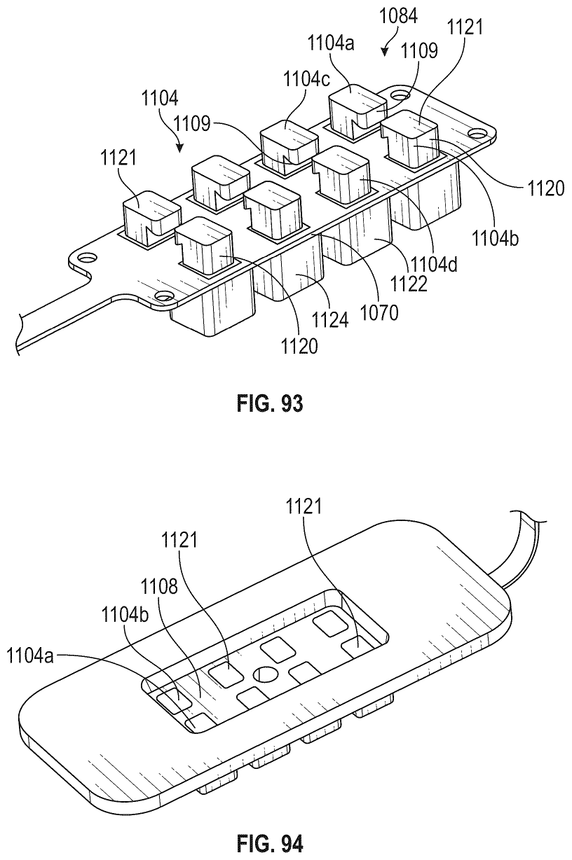

Referring to FIGS. 86-88, the sensor module 1060 may include the elongate sensor 1062 and may include a body 1080 molded upon at least a portion of the elongate sensor 1062. The body 1080 may comprise a housing or elongate sensor housing that houses the proximal end portion 1066 (marked in FIG. 89) of the elongate sensor 1062. The housing may include multiple components in examples, each molded upon the elongate sensor 1062 in a similar manner as with the analyte sensor system or module represented in FIGS. 71-80. For example, the housing may include a frame 1082, one or more electrical contacts 1084 (marked in FIG. 93), and a sealing member 1086 (marked in FIG. 87). The frame 1082 may be configured similarly as the frame 974 shown in FIG. 71 unless stated otherwise. The one or more electrical contacts 1084 may be configured similarly as the electrical contacts 975a, b shown in FIG. 77 unless stated otherwise. The sealing member 1086 may be configured similarly as the sealing member 1034 shown in FIG. 72 unless stated otherwise.

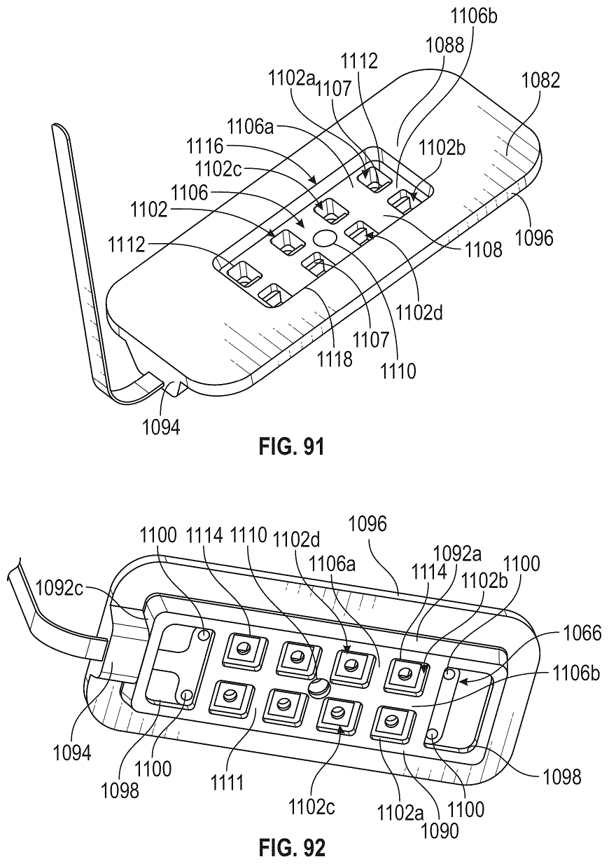

The frame 1082 is shown in isolation in FIGS. 91 and 92 molded upon the proximal end portion 1066 of the elongate sensor 1062. The frame 1082 may be molded upon the elongate sensor 1062 in a similar manner as other forms of frames disclosed herein. The frame 1082 may be molded upon the elongate sensor 1062 to form a seal upon the outer surface of the elongate sensor 1062.

The frame 1082 may include an upper outer surface 1088, a lower outer surface 1090 (marked in FIG. 92) and one or more outer side surfaces 1092a-d (marked in FIGS. 86-88). The surfaces of the frame 1082 may include the features of the corresponding surfaces of the frame 974 shown in FIGS. 74 and 75 unless stated otherwise.

The upper outer surface 1088 may comprise a flat or planar surface in examples. The upper outer surface 1088 may form the upper outer surface of the body 1080 or housing in examples. The outer side surfaces 1092a-d may comprise angled surfaces that angle radially inward in a direction of the frame 1082 from the upper outer surface 1088 towards the lower outer surface 1090. The angle or taper of the outer side surfaces 1092a-d may allow for improved mating with a receiving portion of an external structure such as a socket. For example, the external structure may have a corresponding angle or taper that may allow for improved alignment and mating between the body 1080 and the external structure. In examples, the outer side surface 1092c may include a mating feature 1094 that includes the features of the mating feature 982 of the body 972 shown in FIG. 74.

The body 1080 includes a flange 1096 that protrudes radially outward from the body 1080 and extends circumferentially about the body 1080. The flange 1096 is a part of the frame 1082 and forms the upper outer surface 1088 of the frame 1082. The flange 1096 protrudes radially outward from the outer side surfaces 1092a-d of the frame 1082. The flange 1096 includes the same features and provides the same function as the flange 984 of the body 972. For example, the flange 1096 may be configured for bonding with at least a portion of a socket configured to receive the body 1080. In examples, the flange 1096 may be excluded.

Referring to FIG. 92, the lower outer surface 1090 may face opposite the upper outer surface 1088. The lower outer surface 1090 may include one or more securing features 1098 that may be utilized for securing the sealing member 1086 or other forms of sealing members or fill material to the frame 1082. The securing features 1098, for example, may comprise recesses or cavities configured to receive the material forming the sealing member 1086.

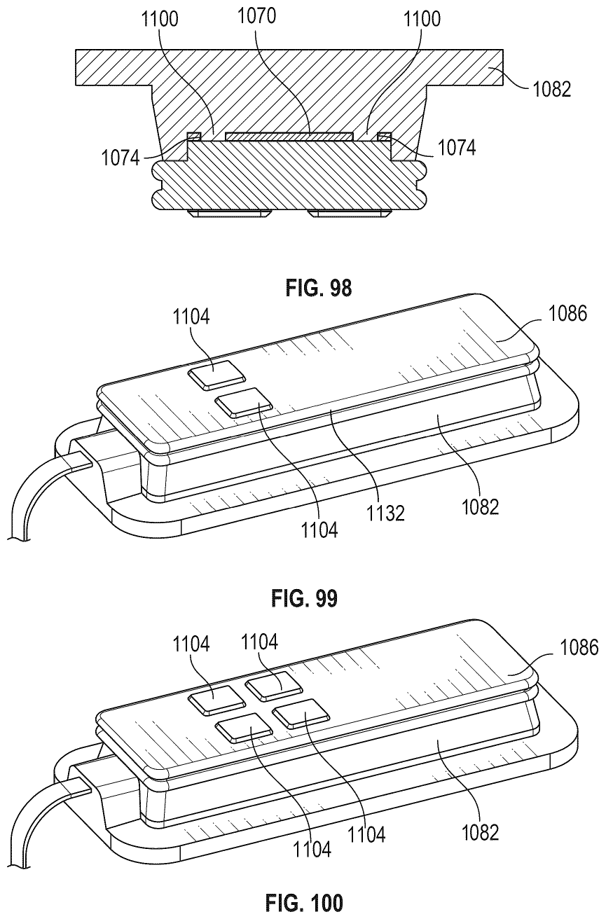

In examples, the frame 1082 may include securing features 1100 in the form of material passing through the openings 1074 of the connection portion 1070 (which may comprise openings configured to receive material of the body 1080 for securing the connection portion 1070 to the body 1080). The securing features 1100 may pass through the openings 1074 during the molding process to secure the connection portion 1070 to the frame 1082 (as shown in the cross sectional view of FIG. 98). Other configurations may be utilized in examples.

The frame 1082 may include a plurality of interior chambers 1102 in examples. A first interior chamber 1102a and second interior chamber 1102b are marked in FIG. 91, although a greater or lesser number of interior chambers may be utilized in examples as desired. For example, a third interior chamber 1102c and a fourth interior chamber 1102d are also marked in FIG. 91. Eight interior chambers 1102 are shown in FIG. 91, which may each correspond to a respective position of a sensor electrical contact 1072 as shown in FIG. 89.

Each interior chamber 1102 may be configured to receive a respective one of the sensor electrical contacts 1072 marked in FIG. 89 (with the first sensor electrical contact 1072a positioned within the first interior chamber 1102a and the second sensor electrical contact 1072b positioned within the second interior chamber 1102b). Further, each interior chamber 1102 may be configured to receive a respective one of the electrical contacts 1104 shown in FIG. 93 (with the first electrical contact 1104a positioned within the first interior chamber 1102a, and the second electrical contact 1104b positioned within the second interior chamber 1102b).

The interior chambers 1102 may be spaced from each other in a longitudinal direction and/or in a lateral direction. The first interior chamber 1102a may be spaced from the third interior chamber 1102c in a longitudinal direction along a length of the proximal end portion 1066 of the elongate sensor 1062. The second interior chamber 1102b may be spaced from the first interior chamber 1102a in a lateral direction that is lateral relative to the length of the proximal end portion 1066 of the elongate sensor 1062. Combinations of longitudinal and lateral spacing corresponding to the positions of the sensor electrical contacts 1072 may be utilized.

In examples, each interior chamber 1102 may include a securing feature 1107 (marked in FIG. 97) that may be configured to engage a respective coupling member 1109 of each electrical contact 1104. The securing feature 1107 may comprise an angled recess configured to receive a coupling member 1109 in the form of a protrusion (e.g., a hook, a flange, among other forms of protrusions). The coupling member 1109 may be molded to have a shape that matches the shape of the securing feature 1107. In examples, the securing features 1107 may be excluded.

The frame 1082 may include one or more dividers 1106 that may divide the interior chambers 1102 from each other. The frame 1082 may include one or more dividers that extend laterally, lateral relative to the length of the proximal end portion 1066 of the elongate sensor 1062 (for example the divider 1106a marked in FIG. 91). The laterally extending dividers (e.g., divider 1106a) may separate the longitudinally adjacent interior chambers (e.g., chambers 1102a and 1102c). The frame 1082 may include one or more dividers that extend longitudinally along the length of the proximal end portion 1066 of the elongate sensor 1062 (for example, the divider 1106b marked in FIG. 91). The longitudinally extending dividers (e.g., divider 1106b) may separate the laterally adjacent interior chambers (e.g., chambers 1102a, 1102b) from each other. Multiple dividers 1106 may be utilized as shown in FIG. 91 to divide the interior chambers 1102 as desired. The dividers 1106 may serve to provide electrical and mechanical isolation between the electrical contacts 1104 (marked in FIG. 93). The dividers 1106 may include an upper surface 1108 and a lower surface 1111 (marked in FIG. 92). In examples, the dividers 1106 may not extend through the connection portion 1070, yet may be formed on opposite sides of the connection portion 1070 during the molding process.

The frame 1082 may be molded onto the connection portion 1070 such that the connection portion 1070 is configured to be contacted by the electrical contacts 1104 in a direction transverse or perpendicular to the plane of the planar substate of the connection portion 1070. The frame 1082 may be molded onto the connection portion 1070 with the connection portion 1070 held in a plane extending parallel to the plane of the skin of the host in examples, and parallel with the planes of the openings 1112, 1114 (and the plane of the main upper opening 1118).

The frame 1082 may be molded onto the connection portion 1070 and onto a proximal section 1071 (marked in FIG. 89) of the insertion portion 1068 (that is proximal to the bend 1073 of the insertion portion 1068). The distal section 1075 of the insertion portion 1068 may be positioned distal of the bend 1073 and exterior of the frame 1082.

In examples, the frame 1082 may include an opening 1110 forming a flow channel for material to pass from the upper surface 1108 to the lower surface 1111. The material may comprise material forming the sealing member 1086 or other forms of sealing members or fill material for the frame 1082. The flow channel may allow for flow during a molding process between the upper surface 1108 and the lower surface 1111. The opening 1110 of the frame 1082 may correspond to the position of the opening 1076 of the connection portion 1070 (marked in FIG. 89) such that material may flow through the opening 1076.

In examples, each interior chamber 1102 may include a respective upper opening 1112 and a lower opening 1114. The upper openings 1112 may be positioned on the upper surface 1108 and the lower openings 1114 may be positioned on the lower surface 1111. The lower openings 1114 may expose the electrical contacts 1104 for electrical connection. The lower openings 1114 may allow for electrical connection of the sensor electrical contacts 1072 with electrical contacts of a sensor electronics module.

The interior chambers 1102 may each comprise sub-chambers of an interior chamber 1116 or main interior chamber (marked in FIG. 91) of the frame 1082. The main interior chamber 1116 may extend from the upper openings 1112 of the interior chambers 1102 to a main upper opening 1118 of the frame 1082. The main upper opening 1118 may be positioned on the upper outer surface 1088 of the frame 1082. The interior chambers 1102 may be continuous with the main interior chamber 1116.

FIG. 93 illustrates the electrical contacts 1104 in isolation, having been molded to the connection portion 1070. Each electrical contact 1104 may be molded to the connection portion 1070 in a similar manner as the electrical contacts 960a, b shown in FIGS. 69 and 70. For example, each electrical contact 1104 may be molded at a location of a respective sensor electrical contact 1072. In examples, each electrical contact 1104 may be molded to extend through a respective one of the openings (exemplary openings 1078a-d are marked in FIG. 89) of the respective sensor electrical contact 1072. Each electrical contact 1104 may surround the respective sensor electrical contact 1072. Each electrical contact 1104 may extend transverse or perpendicular to the opening 1078a-d and to the plane of the planar substrate of the connection portion 1070. Each electrical contact 1104 accordingly may include an upper portion 1120 for being positioned on an upper side of the connection portion 1070 and a lower portion 1122 for being positioned on a lower side of the connection portion 1070. The portions 1120, 1122 may each be sized larger than the respective openings 1078a-d. A neck portion or narrow portion may extend through the opening 1078a-d, and being sized smaller (e.g., a smaller diameter) than the respective portions 1120, 1122. The portions 1120, 1122 may contact and overlay corresponding upper and lower surfaces of the sensor electrical contacts 1072. In examples, the upper portions 1120 may include the coupling member 1109 for coupling with the securing feature 1107 (as shown in the cross sectional view of FIG. 97)

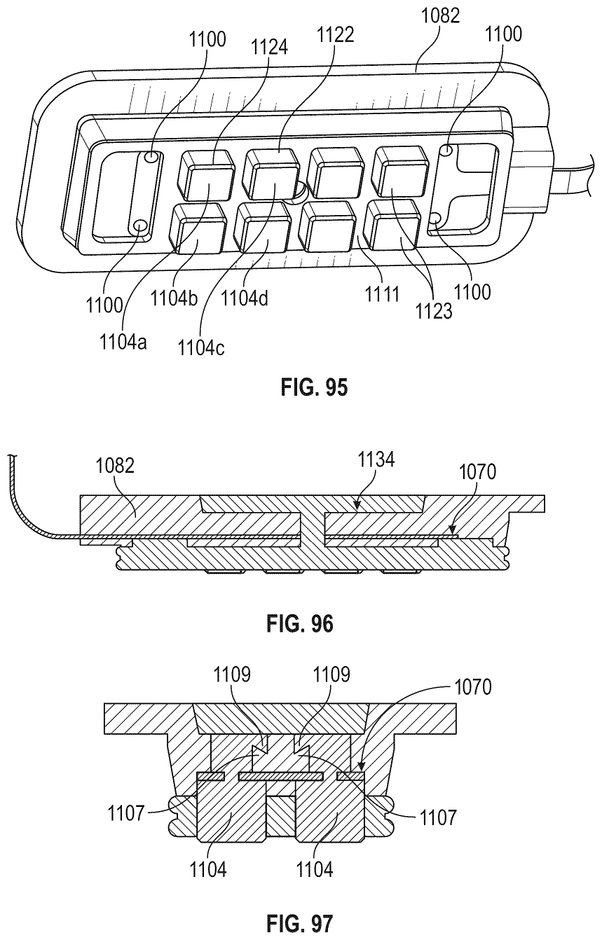

FIGS. 94 and 95 illustrate the one or more electrical contacts 1104 within the frame 1082 and having been molded to the proximal end portion 1066 of the elongate sensor 1062. As discussed regarding the analyte sensor system or module represented in FIGS. 71-80, the electrical contacts 1104 may either be molded initially, with the frame 1082 molded thereafter, or the frame 1082 may be molded initially, with the electrical contacts 1104 molded into the frame 1082. The body 1080 may comprise a multi-shot overmolding upon the elongate sensor 1062. The electrical contacts 1104 are shown to be molded with respective upper outer surfaces 1121, lower outer surfaces 1123 (marked in FIG. 95), and respective outer side surfaces 1124.

Referring to FIG. 94, the respective upper outer surfaces 1121 of the electrical contacts 1104 may be flush with the upper surface 1108 in examples. Referring to FIG. 95, the lower outer surfaces 1123 of the electrical contacts 1104 may protrude from the lower surface 1111 in examples. Other configurations may be utilized as desired.

In a configuration as shown in FIGS. 94 and 95, the sealing member or fill material may be molded to produce a resulting configuration shown in FIGS. 86-88. The sealing member 1086 shown in FIG. 87 is formed in such a molding process. The sealing member 1086 may be configured similarly as the sealing member 1034 represented in FIG. 72. For example, the sealing member 1086 may comprise the bottom layer of the body 1080. The sealing member 1086 may form an outer surface of the body 1080 (the lower outer surface 1130 of the body 1080). The sealing member 1086 may cover the lower outer surface 1090 of the frame 1082.

The lower outer surfaces 1123 of the respective electrical contacts 1104 may protrude from the lower outer surface 1130 of the body 1080 as shown in FIG. 87. Such a feature may allow for surface-to-surface connection with external electrical contacts as disclosed herein. The sealing member 1086 may seal such a connection (e.g., a face seal). In examples, the height of the lower outer surfaces 1123 of the respective electrical contacts 1104 may be reduced such that the sealing member 1086 entirely covers the lower outer surfaces 1123 of the respective electrical contacts 1104 (as represented in FIGS. 34-36 for example). In such a configuration the sealing member 1086 may be penetrated by electrical contacts as disclosed herein for electrical connection. Other configurations may be utilized as desired.

Similar to the configuration of the sealing member 1034 represented in FIG. 72, the sealing member 1086 may be part of other forms of sealing members and/or fill material that may be molded. In examples, the body 1080 includes a sealing member 1132 (marked in FIG. 87) configured to form a radial seal. The sealing member 1132 may be configured similarly as, and include the functions of the sealing member 1040 marked in FIG. 71. In examples, a sealing member similar to the sealing member 979 shown in FIG. 72 may be utilized. For example, a sealing member forming one or more ribs and/or grooves for forming a seal with a surface may be utilized. The sealing member may extend around all of the lower outer surfaces 1123 of the electrical contacts 1104 or individual sealing members may extend around each of the lower outer surfaces 1123 of the electrical contacts 1104. Other configurations may be utilized as desired.

The sealing members may be formed as a part of fill material that fills the main interior chamber 1116. The fill material may pass through the opening 1110 (and the opening 1076 of the connection portion 1070) to pass from the upper surface 1108 to the lower surface 1111. The sealing members may be formed in such a molding process.

An upper portion 1134 (marked in FIG. 86) of the fill material may fill the main interior chamber 1116. The lower portion of the fill material positioned on an opposite side of the dividers 1106 may form the sealing members 1086, 1132.

FIG. 96, for example, illustrates a cross sectional view along line VII-VII in FIG. 86. The fill material fills the main interior chamber 1116 to form the sealing members 1086, 1132 on the opposite side of the dividers 1106.

FIG. 97 illustrates a cross sectional view along line VIII-VIII in FIG. 87. FIG. 98 illustrates a cross sectional view along line IX-IX in FIG. 87. The securing features 1100 are shown to extend through the connection portion 1070.

Various other configurations may be utilized as desired.

FIG. 99, for example, illustrates a variation in which the number of electrical contacts 1104 varies from a configuration as shown in FIG. 87. In the variation of FIG. 99, two electrical contacts 1104 may be utilized, and a corresponding number of two sensor electrical contacts 1072 may be utilized. Such a configuration may be a single analyte or single parameter sensing configuration. For example, a minimum number of sensor electrical contacts 1072 (and electrical contacts 1104) for single analyte sensing (such as a glucose sensor) may be two contacts. A corresponding number of two electrodes on the insertion portion may be utilized (similar to a configuration shown in FIG. 64). A greater number of sensor electrical contacts 1072 (and electrical contacts 1104) may be utilized for multi-analyte sensing or single analyte sensing (with a single elongate sensor).

For example, three sensor contacts 1072 (and three electrical contacts 1104) may be utilized for single analyte sensing (or multi-analyte sensing). A corresponding number of electrodes may be utilized. In multi-analyte sensing, the elongate sensor 1062 may be configured to detect a first analyte and detect a second analyte that is different than the first analyte. The sensor electrical contacts 1072 may be configured to provide electrical signals corresponding to the detected first analyte and second analyte. For example, the first analyte may be glucose, and the second analyte may be cortisol or another form of analyte as disclosed herein. Various forms of analytes as disclosed herein may be detected. In examples, other parameters such as internal temperature may be detected in examples. Combinations of analytes and other parameters may be detected.

In examples, four sensor contacts 1072 (and four electrical contacts 1104) may be utilized for single analyte sensing (or multi-analyte sensing) as represented in FIG. 100. A corresponding number of electrodes may be utilized. In examples, a greater number of sensor contacts and electrical contacts may be utilized. In examples, each successive sensor contact and electrical contact over four contacts may be utilized to detect an additional analyte or parameter. For example, six contacts may be utilized to detect three analytes or parameters. Eight contacts may be utilized to detect five analytes or parameters (as represented in FIG. 87).

In examples, each successive sensor contact and electrical contact over three contacts may be utilized to detect an analyte or parameter. For example, six contacts may be utilized to detect four analytes or parameters. Eight contacts may be utilized to detect six analytes or parameters (as represented in FIG. 87). A greater number of analytes or parameters may be detected as desired (e.g., three or more analytes or parameters, four or more, eight or more, etc.). A corresponding number of electrodes may be utilized with the elongate sensor. In examples, the relationship with analytes and contacts may be varied (e.g., eight contacts utilized to sense two analytes or parameters, among other combinations). Multi-analyte sensing may also be utilized with a configuration of an elongate sensor as shown in FIGS. 108 and 109.

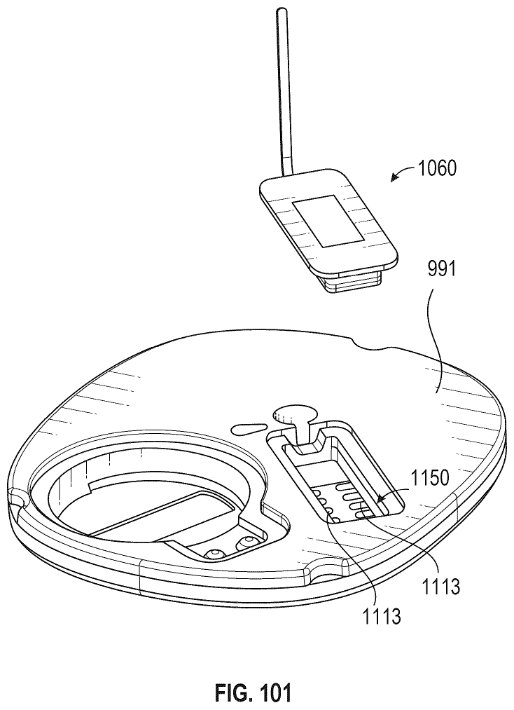

FIGS. 101-104 illustrate a coupling between the sensor module 1060 and a receiving portion or socket 1105. The socket 1105 may comprise any form of socket disclosed herein. The socket 1105 for example, may be configured similarly as the socket 990 shown in FIG. 81.

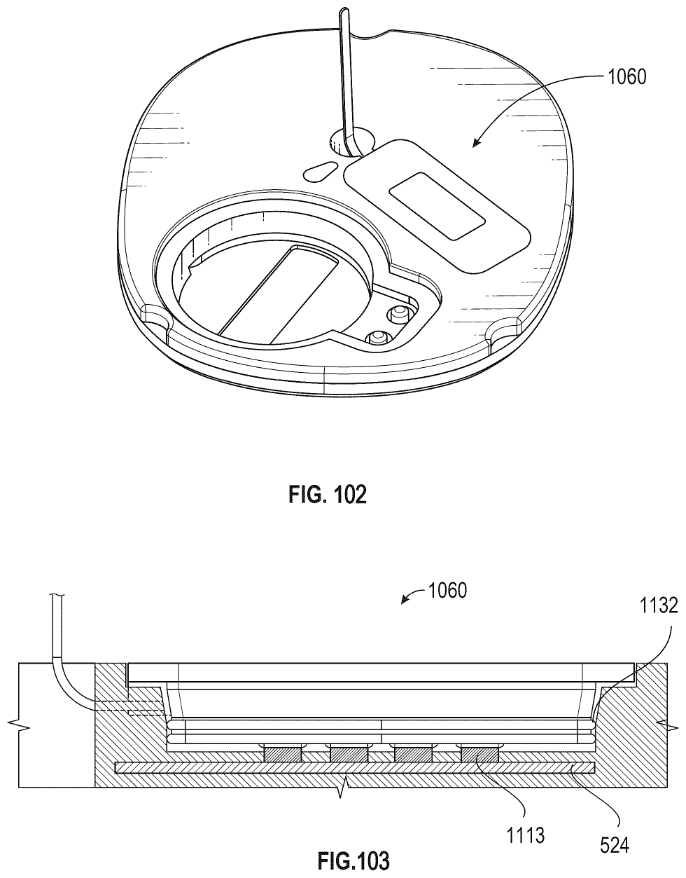

The sensor module 1060 is shown in FIG. 101 spaced from the socket 1105. The sensor module 1060 may be pressed in a direction transverse or perpendicular to a direction of the proximal end portion 1066 of the elongate sensor 1062 towards the socket 1105. The sensor module 1060 may be pressed into the socket 1105 as represented in FIG. 102.

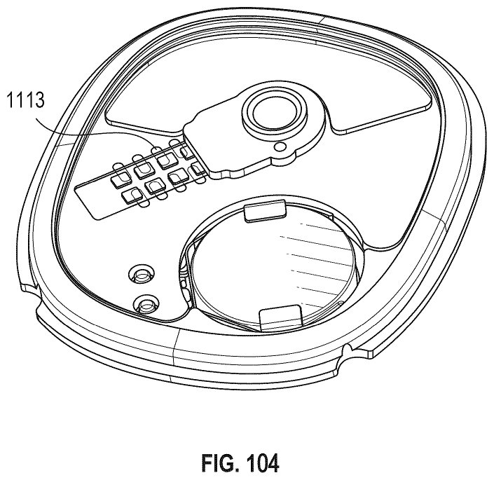

FIG. 103 illustrates a partial cross sectional view of the sensor module 1060 within the socket 1105. A configuration similar to the configuration shown in FIG. 83 results. The sealing member 1132 may contact side walls or side surfaces of the socket 1105 in a similar manner as discussed in regard to FIG. 83. The sensor module 1060 may electrically connect with the external electrical contacts 1113, which may be positioned in the same positions as the electrical contacts of the sensor module 1060. FIG. 104, for example, illustrates an opposite view of the position of the electrical contacts 1113.

Other configurations may be utilized in examples.

In examples, the analyte sensor systems or sensor modules disclosed in regard to FIGS. 86-104 may be utilized with any form of receiving portion or socket as disclosed herein, including any receiving portion or socket disclosed in regard to FIGS. 37-58, among other forms of receiving portions or sockets. The analyte sensor systems or sensor modules disclosed in regard to FIGS. 86-104 may be utilized in any position or orientation disclosed herein, including any receiving position or orientation disclosed in regard to FIGS. 37-58, among other positions or orientations.

The features of any of the examples of FIGS. 86-104 may be utilized solely or in combination with any other example herein.

Other variations may be utilized in examples.

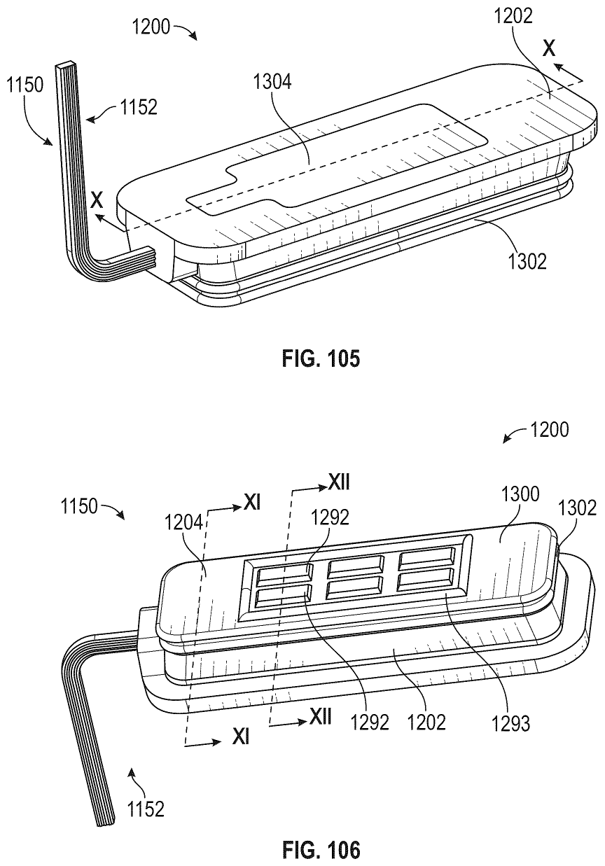

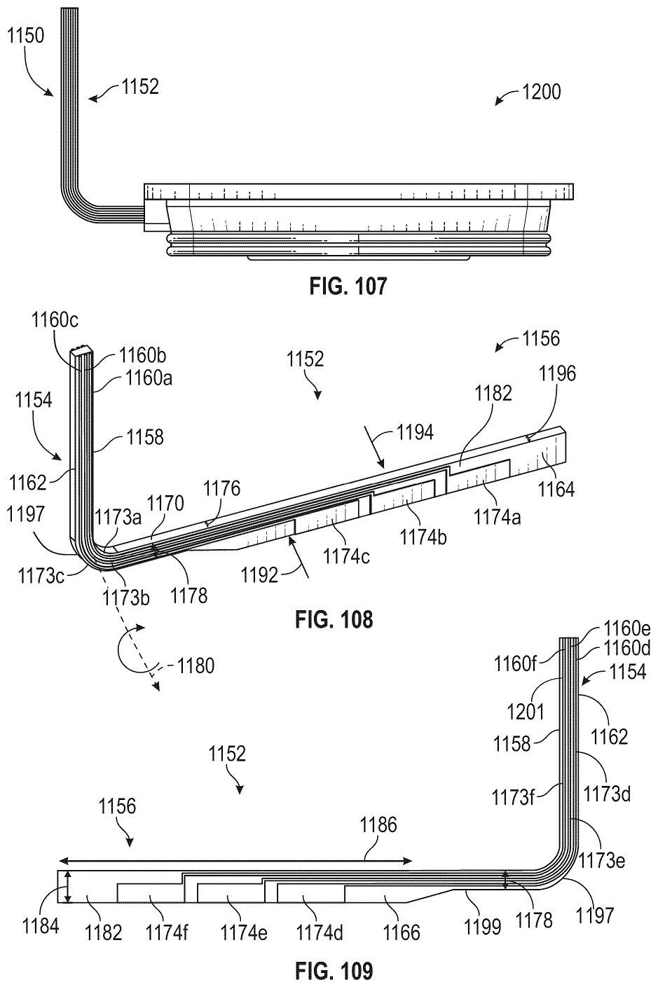

FIGS. 105-107 illustrate a variation of a configuration of the analyte sensor system or modules represented in FIGS. 71-104. The analyte sensor system or sensor module 1150 may include the features of the analyte sensor system or modules represented in FIGS. 71-104 unless stated otherwise.

The sensor module 1150 comprises a configuration of a sensor module adapted for supporting an elongate sensor having a flat or planar sensor. The orientation of the elongate sensor and the direction of the bend differs from a configuration as represented in FIGS. 64-66 for example. The elongate sensor otherwise includes the features of the elongate sensor 902 as represented in FIGS. 64-66 unless stated otherwise.

FIGS. 108 and 109 illustrate an isolated view of the elongate sensor 1152. The elongate sensor 1152 may include a distal end portion 1154 and a proximal end portion 1156. The distal end portion 1154 may be configured to be inserted into skin of a host (an in vivo portion) and the proximal end portion 1156 may be configured to be positioned exterior of the skin of the host (an ex vivo portion). The elongate sensor 1152 may operate in a similar manner as discussed regarding the elongate sensor 137 (for example as discussed in regard to FIGS. 1-3D). The elongate sensor 1152, however, may comprise a planar sensor having a planar substrate that components of the elongate sensor 1152 are positioned upon.

For example, the distal end portion 1154 of the elongate sensor 1152 includes an insertion portion 1158. The insertion portion 1158 is configured for insertion into the skin of a host. The insertion portion 1158 may include a planar substrate and may include electrodes 1160a-f for detecting an analyte in a similar manner as discussed regarding the elongate sensor 137. The electrodes 1160a-f are positioned on an elongated strip 1162 of the planar substrate of the insertion portion 1158 (such as a thin, flat, polymer flex circuit).

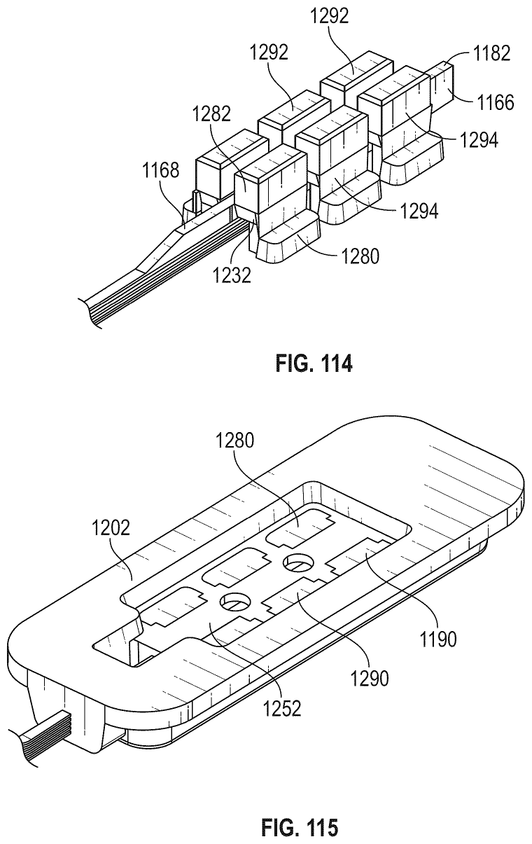

The electrodes 1160a-f may be positioned on opposite sides 1164, 1166 of the insertion portion 1158 in examples. For example, the electrodes 1160a-c may be positioned on a first side 1164 of the insertion portion 1158. The electrodes 1160d-f may be positioned on a second side 1166 of the insertion portion 1158 that faces opposite the first side 1164. The sides 1164, 1166 may comprise lateral sides configured to face laterally when the insertion portion 1158 is inserted into the skin of the host. The insertion portion 1158 may include a lower side 1168 (marked in FIG. 114) and an upper side 1170 (marked in FIG. 108) that may face opposite each other and may extend between the respective lateral sides 1164, 1166.

Circuit traces 1173a-f may extend along the length of the elongated strip 1162 proximally to respective sensor electrical contacts 1174a-f positioned at the proximal end portion 1156 of the elongate sensor 1152. The electrodes 1160a-f are positioned on the elongated strip 1162 to be disposed within the skin of the host for detecting the analyte therein.

In examples, the insertion portion 1158 has a thickness 1176 (marked in FIG. 108) and a width 1178 that is greater than the thickness 1176. The insertion portion 1158 is configured to bend (marked with the curved arrow in FIG. 108) about an axis 1180 (marked in FIG. 108) that extends in the dimension that is transverse or perpendicular to the dimension that the width 1178 extends along. The axis 1180 extends in the dimension that the thickness 1176 extends along. The insertion portion 1158 is shown to extend in an upward direction in FIG. 108, yet the direction of bend may alternatively be in a downward direction in examples. The elongate sensor 1152 may be a bent elongate sensor.

The proximal end portion 1156 of the elongate sensor 1152 includes a connection portion 1182 including a planar substrate and having the sensor electrical contacts 1174a-f positioned thereon. The connection portion 1182 may comprise an electrical connection portion of the elongate sensor 1152 for electrical connection with other electrical contacts (e.g., for transmitting an electrical signal corresponding to the detected analyte as disclosed herein). In examples, the sensor electrical contacts 1174a-c may be spaced from each other in a longitudinal direction along a length of the connection portion 1182 as shown in FIG. 108. The sensor electrical contacts 1174d-f may be spaced from each other in a longitudinal direction along a length of the connection portion 1182 on the opposite side 1166 of the connection portion 1182 as shown in FIG. 109. The sensor electrical contacts 1174a-c may be aligned with each other in the longitudinal direction as shown in FIG. 108 and the electrical contacts 1174d-f may be aligned with each other in the longitudinal direction as shown in FIG. 109. The sensor electrical contacts 1174a-c may be spaced from the sensor electrical contacts 1174d-f in a lateral direction that is lateral relative to the length of the connection portion 1182 (e.g., on the opposite side of the connection portion 1182).

The connection portion 1182 may have a width 1184 and a length 1186, with the length 1186 being greater than the width 1184. The width 1184 of the connection portion 1182 may extend in the width dimension of the insertion portion 1158. As such, the connection portion 1182 may extend in the dimension that the width 1178 of the insertion portion 1158 extends along. The connection portion 1182 may extend in a plane that is parallel or coextensive with the plane of the insertion portion 1158. The connection portion 1182 may be configured to receive one or more electrical contacts (such as electrical contacts 1190 shown in FIG. 113) in directions 1192, 1194 (marked in FIG. 108) that are transverse (e.g., perpendicular) to the dimension that the connection portion 1182 extends in.

The connection portion 1182 may have a thickness 1196 (marked in FIG. 108) that may be the same as the thickness 1176 of the insertion portion 1158. The same thickness may result from the elongate sensor 1152 being formed from a unitary sheet of materials having uniform thickness, which may be stamped, die cut, or laser cut (among other forms of separation) to cut out the shape of the elongate sensor 1152. The insertion portion 1158 may be cut in a shape having the bend 1197 shown in FIGS. 108 and 109. The bend 1197 accordingly may be preformed during manufacture (whereas the bend 1073 shown in FIG. 89 may be formed following a cutting process). In examples, the connection portion 1182 may have a different thickness (e.g., greater or lesser thickness) than the insertion portion 1158. Other configurations may be utilized in examples.

The width 1184 of the connection portion 1182 may be greater than the width 1178 of the insertion portion 1158 as shown in FIG. 109 for example.

The elongate sensor 1152 may include six sensor electrical contacts 1174a-f or in examples, a greater or lesser number of sensor electrical contacts 1174a-f may be utilized in examples as desired. The greater number of sensor electrical contacts 1174a-f may be due to the elongate sensor 1152 being configured to detect multiple different analytes (a multi-analyte sensor). In examples, the elongate sensor 1152 may be configured to detect a single analyte. A greater or lesser number of sensor electrical contacts may be utilized in examples as desired.

Referring to FIGS. 105-107, the sensor module 1150 may include the elongate sensor 1152, and may include a body 1200 molded upon at least a portion of the elongate sensor 1152. The body 1200 may comprise a housing or elongate sensor housing that houses the proximal end portion 1156 (marked in FIG. 108) of the elongate sensor 1152. The housing may include multiple components in examples, each molded upon the elongate sensor 1152 in a similar manner as with the analyte sensor systems or modules represented in FIGS. 71-104. For example, the housing may include a frame 1202, one or more electrical contacts 1190 (marked in FIG. 113), and a sealing member 1204 (marked in FIG. 106). The frame 1202 may be configured similarly as the frame 974 shown in FIG. 71 or the frame 1082 shown in FIG. 86 unless stated otherwise. The one or more electrical contacts 1190 may be configured similarly as the electrical contacts 975a, b shown in FIG. 77 or the electrical contacts 1104 shown in FIG. 93 unless stated otherwise. The sealing member 1204 may be configured similarly as the sealing member 1034 shown in FIG. 72 or the sealing member 1086 shown in FIG. 87 unless stated otherwise.

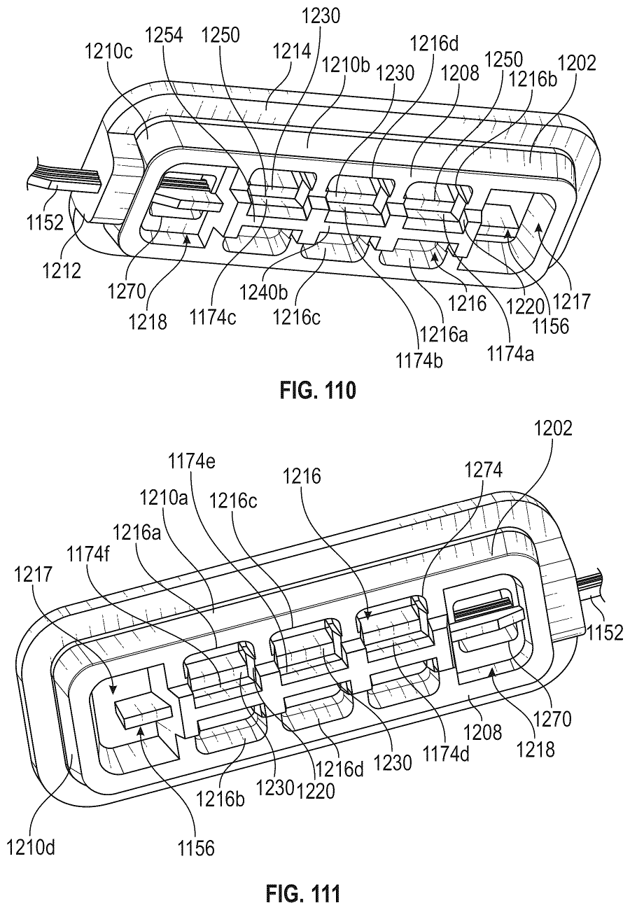

The frame 1202 is shown in isolation in FIGS. 110-112 molded upon the proximal end portion 1156 of the elongate sensor 1152. The frame 1202 may be molded upon the elongate sensor 1152 in a similar manner as other forms of frames disclosed herein. The frame 1202 may be molded upon the elongate sensor 1152 to form a seal upon the outer surface of the elongate sensor 1152.

The frame 1202 may include an upper outer surface 1206, a lower outer surface 1208 (marked in FIGS. 110 and 111) and one or more outer side surfaces 1210a-d (marked in FIGS. 110 and 111). The surfaces of the frame 1202 may include the features of the corresponding surfaces of the frame 974 shown in FIGS. 74 and 75 or may include the features of the corresponding surfaces of the frame 1082 shown in FIGS. 91 and 92 unless stated otherwise.

The upper outer surface 1206 may comprise a flat or planar surface in examples. The upper outer surface 1206 may form the upper outer surface of the body 1200 or housing in examples. The outer side surfaces 1210a-d may comprise angled surfaces that angle radially inward in a direction of the frame 1202 from the upper outer surface 1206 towards the lower outer surface 1208. The angle or taper of the outer side surfaces 1210a-d may allow for improved mating with a receiving portion of an external structure such as a socket. For example, the external structure may have a corresponding angle or taper that may allow for improved alignment and mating between the body 1200 and the external structure. In examples, the outer side surface 1210c may include a mating feature 1212 that includes the features of the mating feature 982 shown in FIG. 74 or the mating feature 1094 shown in FIG. 91.

The body 1200 includes a flange 1214 that protrudes radially outward from the body 1200 and extends circumferentially about the body 1200. The flange 1214 is a part of the frame 1202 and forms the upper outer surface 1206 of the frame 1202. The flange 1214 protrudes radially outward from the outer side surfaces 1210a-d of the frame 1202. The flange 1214 includes the same features and provides the same function as the flange 984 shown in FIG. 74 or the flange 1096 shown in FIG. 91. For example, the flange 1214 may be configured for bonding with at least a portion of a socket configured to receive the body 1200. In examples, the flange 1214 may be excluded.

Referring to FIGS. 110 and 111, the lower outer surface 1208 may face opposite the upper outer surface 1206. The lower outer surface 1208 may include one or more securing features 1217, 1218 that may be utilized for securing the sealing member 1204 or other forms of sealing members or fill material to the frame 1202. The securing features 1217, 1218, for example, may comprise recesses or cavities configured to receive the material forming the sealing member 1204.

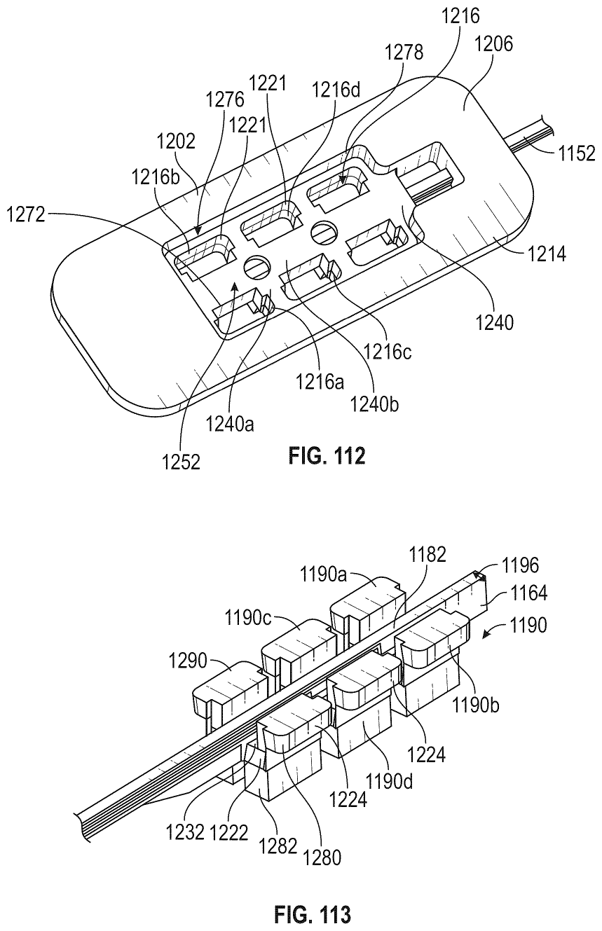

The frame 1202 may include a plurality of interior chambers 1216 in examples. A first interior chamber 1216a and second interior chamber 1216b are marked in FIG. 112, although a greater or lesser number of interior chambers may be utilized in examples as desired. For example, a third interior chamber 1216c and a fourth interior chamber 1216d are also marked in FIG. 112. Six interior chambers 1216 are shown in FIG. 112, which may each correspond to a respective position of an electrical contact 1190 as shown in FIG. 113.

Each interior chamber 1216 may be configured to receive a respective one of the sensor electrical contacts 1174a-f marked in FIGS. 108 and 109 (with the sensor electrical contact 1174f positioned within the first interior chamber 1216a, the sensor electrical contact 1174a positioned within the second interior chamber 1216b, and the sensor electrical contact 1174e positioned within the third interior chamber 1216c). Further, each interior chamber 1216 may be configured to receive a respective one of the electrical contacts 1190 shown in FIG. 113 (with the electrical contact 1190a positioned within the first interior chamber 1216a, the second electrical contact 1190b positioned within the second interior chamber 1216b, the third electrical contact 1190c positioned within the third interior chamber 1216c, and the fourth electrical contact 1190d positioned within the fourth interior chamber 1216d).

The interior chambers 1216 may be spaced from each other in a longitudinal direction and/or in a lateral direction. The first interior chamber 1216a may be spaced from the third interior chamber 1216c in a longitudinal direction along a length of the proximal end portion 1156 of the elongate sensor 1152. The second interior chamber 1216b may be spaced from the first interior chamber 1216a in a lateral direction that is lateral relative to the length of the proximal end portion 1156 of the elongate sensor 1152. Combinations of longitudinal and lateral spacing corresponding to the positions of the sensor electrical contacts 1174a-f may be utilized.

In examples, each interior chamber 1216 may include a securing feature in the form of tapered surfaces 1220, 1221 that may be configured to engage with a corresponding tapered surface 1222, 1224 (marked in FIG. 113) of the electrical contacts 1190. Corresponding lower tapered surfaces 1220, 1222, and upper tapered surfaces 1221, 1224 may be utilized. The angle of the lower tapered surfaces 1220, 1222, and upper tapered surfaces 1221, 1224 may be opposite to each other, to wedge the electrical contacts 1190 in position to resist both a force in an upward direction and a force in a lower direction.

In examples, each interior chamber 1216 may include a supporting surface 1230 that may support the electrical contacts 1190 from a force in an upward direction. The supporting surface 1230 may extend in a lateral direction, for a protruding shelf 1232 (marked in FIG. 113) of the respective electrical contacts 1190 to press against. A cross sectional view of the arrangement is shown in FIG. 119.

The frame 1202 may include one or more dividers 1240 that may divide the interior chambers 1216 from each other. The frame 1202 may include one or more dividers that extend laterally, lateral relative to the length of the proximal end portion 1156 of the elongate sensor 1152 (for example the divider 1240a marked in FIG. 112). The laterally extending dividers (e.g., divider 1240a) may separate the longitudinally adjacent interior chambers (e.g., chambers 1216a and 1216c). The frame 1202 may include one or more dividers that extend longitudinally along the length of the proximal end portion 1156 of the elongate sensor 1152 (for example, the divider 1240b marked in FIG. 112). The longitudinally extending divider 1240b may separate the laterally adjacent interior chambers (e.g., chambers 1216a, 1216b) from each other.

The proximal end portion 1156 of the elongate sensor 1152 may extend longitudinally through the divider 1240b. The divider 1240b may include openings 1250 (marked in FIG. 110) that may expose the respective sensor electrical contacts 1174a-f to the respective interior chambers 1216. The sensor electrical contacts 1174a-f may be exposed for electrical connection with the respective electrical contacts 1190.

The dividers 1240 may serve to provide electrical and mechanical isolation between the electrical contacts 1190 (marked in FIG. 113). The dividers 1240 may include an upper surface 1252 (marked in FIG. 112) and a lower surface 1254 (marked in FIG. 110). The divider 1240b may be formed on opposite lateral sides 1164, 1166 (marked in FIGS. 108, 109) of the connection portion 1182 during the molding process.

The frame 1202 may be molded onto the connection portion 1182 such that the connection portion 1182 is configured to be contacted laterally by the electrical contacts 1190. The frame 1202 may be molded onto the connection portion 1182 with the connection portion 1182 held in a plane extending perpendicular to the plane of the skin of the host in examples, and perpendicular to the planes of the openings 1272, 1274 (and the plane of the main upper opening 1278).

The frame 1202 may be molded onto the connection portion 1182 and a proximal section 1199 (marked in FIG. 109) of the insertion portion 1158 (that is proximal to the bend 1197 of the insertion portion 1158). The distal section 1201 of the insertion portion 1158 may be positioned distal of the bend 1197 and exterior of the frame 1202.

In examples, the frame 1202 may include an opening 1270 forming a flow channel for material to pass from the upper surface 1252 to the lower surface 1254. The material may comprise material forming the sealing member 1204 or other forms of sealing members or fill material for the frame 1202. The flow channel may allow for flow between the upper surface 1252 and the lower surface 1254 during a molding process. The flow channel may extend around the elongate sensor 1152 in examples (as shown in FIG. 111 for example).

In examples, each interior chamber 1216 may include a respective upper opening 1272 and a lower opening 1274. The upper openings 1272 may be positioned on the upper surface 1252 and the lower openings 1274 may be positioned on the lower surface 1254. The lower openings 1274 may expose the electrical contacts 1190 for electrical connection. The lower openings 1274 may allow for electrical connection of the sensor electrical contacts 1174a-f with electrical contacts of a sensor electronics module.

The interior chambers 1216 may each comprise sub-chambers of an interior chamber 1276 or main interior chamber (marked in FIG. 112) of the frame 1202. The main interior chamber 1276 may extend from the upper openings 1272 of the interior chambers 1216 to a main upper opening 1278 of the frame 1202. The main upper opening 1278 may be positioned on the upper outer surface 1206 of the frame 1202. The interior chambers 1216 may be continuous with the main interior chamber 1276.

FIGS. 113 and 114 illustrate the electrical contacts 1190 in isolation, adjacent to the connection portion 1182. Each electrical contact 1190 may be formed in a molding process, similar to the electrical contacts 960a, b shown in FIGS. 69 and 70. Each electrical contact 1190 may be molded at a location of a respective sensor electrical contact 1174a-f.

In examples, each electrical contact 1190 may be molded to include an upper portion 1280 and a lower portion 1282. The upper portion 1280 may include the tapered surface 1224. The lower portion 1282 may include the tapered surface 1222 and the protruding shelf 1232. The lower portion 1282 may protrude laterally inward towards the connection portion 1182 for lateral contact with a respective one of the sensor electrical contacts 1174a-f. The direction of contact is transverse or perpendicular to the width dimension of the connection portion 1182. The electrical contacts 1190 may be molded to have the lateral contact with the sensor electrical contacts 1174a-f.

In examples, two columns of electrical contacts 1190, laterally spaced from each other, may be provided. Each column may be positioned on a respective side 1164, 1166 of the connection portion 1182. Three rows of electrical contacts 1190 may be provided, for contact with a respective sensor electrical contact 1174a-f. In examples, other configurations (e.g., electrical contacts 1190 only on one side of the connection portion 1182, or a greater or lesser number of electrical contacts 1190) may be utilized as desired.

FIG. 114 illustrates an opposite or inverted view of the electrical contacts 1190 of FIG. 113.

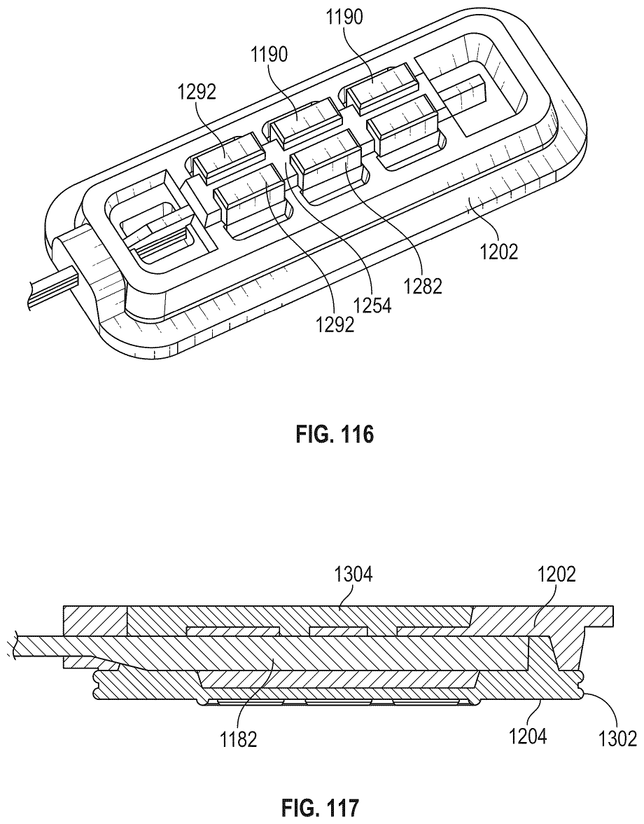

FIGS. 115 and 116 illustrate the one or more electrical contacts 1190 having been molded to the frame 1202. As discussed regarding the analyte sensor system or module represented in FIGS. 71-80, the electrical contacts 1190 may either be molded initially, with the frame 1202 molded thereafter, or the frame 1202 may be molded initially, with the electrical contacts 1190 molded into the frame 1202. The body 1200 may comprise a multi-shot overmolding upon the elongate sensor 1152. The electrical contacts 1190 are shown to be molded with respective upper outer surfaces 1290, lower outer surfaces 1292 (marked in FIG. 114), and respective outer side surfaces 1294 (marked in FIG. 114).

Referring to FIG. 115, the respective upper outer surfaces 1290 of the electrical contacts 1190 may be flush with the upper surface 1252 in examples. Referring to FIG. 116, the lower outer surfaces 1292 of the electrical contacts 1190 may protrude from the lower outer surface 1254 in examples. Other configurations may be utilized as desired.

In a configuration as shown in FIGS. 115 and 116, a sealing member or fill material may be molded to produce a resulting configuration shown in FIGS. 105-107. The sealing member 1204 shown in FIG. 106 is formed in such a molding process. The sealing member 1204 may be configured similarly as the sealing member 1034 represented in FIG. 72 or the sealing member 1086 represented in FIG. 87. For example, the sealing member 1204 may comprise the bottom layer of the body 1200. The sealing member 1204 may form an outer surface of the body 1200 (the lower outer surface 1300 of the body 1200). The sealing member 1204 may cover the lower surface 1254 of the frame 1202.

The lower outer surfaces 1292 of the respective electrical contacts 1190 may protrude from the lower outer surface 1300 of the body 1200 as shown in FIG. 106. Such a feature may allow for surface-to-surface connection with external electrical contacts as disclosed herein. The sealing member 1204 may seal such a connection (e.g., a face seal). In examples, the height of the lower outer surfaces 1292 of the respective electrical contacts 1190 may be reduced such that the sealing member 1204 entirely covers the lower outer surfaces 1292 of the respective electrical contacts 1190 (as represented in FIGS. 34-36 for example). In such a configuration the sealing member 1204 may be penetrated by electrical contacts as disclosed herein for electrical connection. Other configurations may be utilized as desired.

Similar to the configuration of the sealing member 1034 represented in FIG. 72 or the sealing member 1086 represented in FIG. 87, the sealing member 1204 may be part of other forms of sealing members and/or fill material that may be molded. In examples, the body 1200 includes a sealing member 1302 (marked in FIG. 106) configured to form a radial seal. The sealing member 1302 may be configured similarly as, and include the functions of the sealing member 1040 marked in FIG. 71 or the sealing member 1132 marked in FIG. 87.

In examples, a sealing member 1293 may extend around all of the lower outer surfaces 1292 of the electrical contacts 1190. The sealing member may be part of other forms of sealing members and/or fill material. In variations, individual sealing members may extend around each of the lower outer surfaces 1292 of the electrical contacts 1190. Other configurations may be utilized as desired.

The sealing members may be formed as a part of fill material that fills the main interior chamber 1276. The fill material may pass through the opening 1270 (marked in FIG. 111) to pass from the upper surface 1252 to the lower surface 1254. The sealing members may be formed in such a molding process.

An upper portion 1304 (marked in FIG. 117) of the fill material may fill the main interior chamber 1276. The lower portion of the fill material positioned on an opposite side of the dividers 1240 may form the sealing members 1204, 1302.

FIG. 117, for example, illustrates a cross sectional view along line X-X in FIG. 105. The fill material fills the main interior chamber 1276 to form the sealing members 1204, 1302 on the opposite side of the dividers 1240.

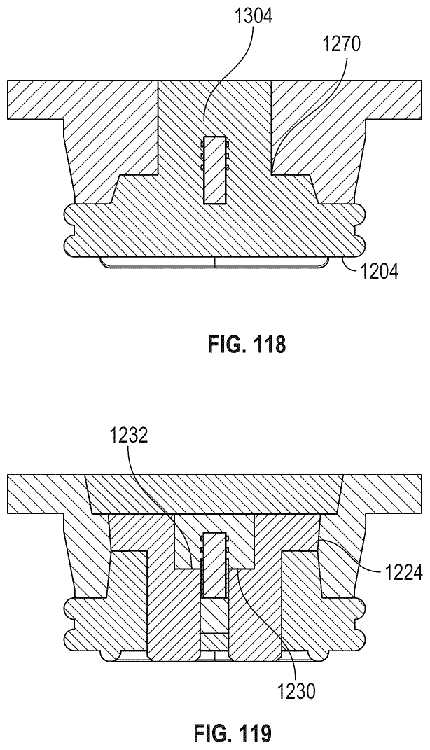

FIG. 118 illustrates a cross sectional view along line XI-XI in FIG. 106. FIG. 119 illustrates a cross sectional view along line XII-XII in FIG. 106.

Various other configurations may be utilized as desired.

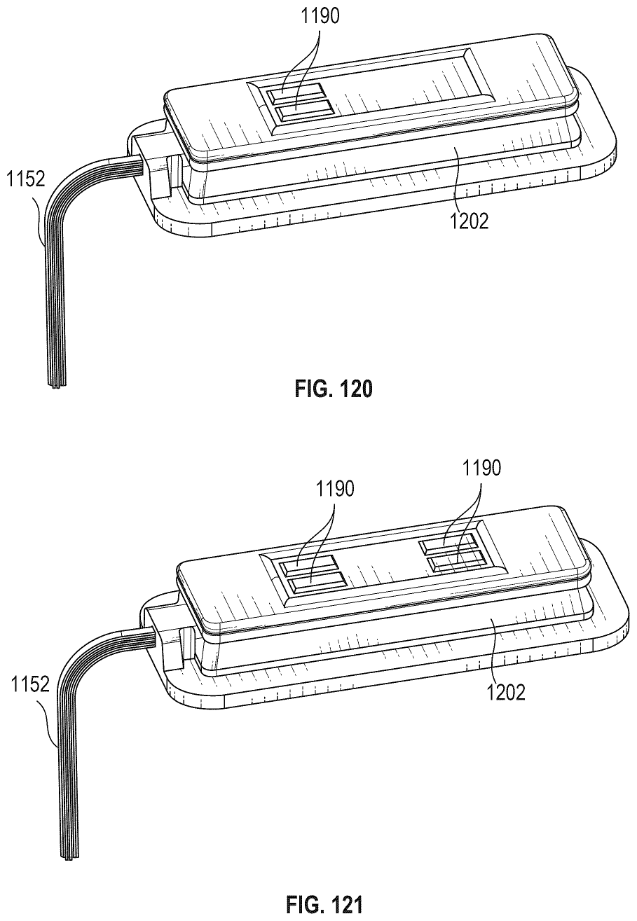

FIG. 120, for example, illustrates a variation in which the number of electrical contacts 1190 varies from a configuration as shown in FIG. 106. In the variation of FIG. 120, two electrical contacts 1190 may be utilized, and a corresponding number of two sensor electrical contacts 1174c, d may be utilized. Such a configuration may be a single analyte or single parameter sensing configuration in a similar manner as discussed regarding FIG. 99. FIG. 121 illustrates a variation in which four electrical contacts 1190 are utilized. Single analyte sensing or multi-analyte sensing (or parameter sensing) may be utilized in a similar manner as disclosed regarding FIGS. 99 and 100. A relationship between a number of analytes or parameters to be detected and the number of contacts disclosed in regard to FIGS. 99 and 100 may be utilized.

In examples, the analyte sensor systems or sensor modules disclosed in regard to FIGS. 105-121 may be utilized with any form of receiving portion or socket as disclosed herein, including any receiving portion or socket disclosed in regard to FIGS. 37-58 or FIGS. 81-83, or FIGS. 101-103, among other forms of receiving portions or sockets. The analyte sensor systems or sensor modules disclosed in regard to FIGS. 105-121 may be utilized in any position or orientation disclosed herein, including any receiving position or orientation disclosed in regard to FIGS. 37-58, among other positions or orientations.

The features of any of the examples of FIGS. 105-121 may be utilized solely or in combination with any other example herein.

In examples, the analyte sensor systems or sensor modules disclosed in regard to FIGS. 71-121 may be coupled with sensor electronics or a wearable device in situ or as part of an application process of the on-skin wearable medical device. For example, the analyte sensor systems or sensor modules may be positioned on the skin of the host initially, and then a receiving portion or socket may be placed upon the analyte sensor system or module. A press fit coupling may result. Electrical connections may be provided between the electrical contacts of the analyte sensor systems or sensor modules and the external electrical contacts. Other configurations may be utilized as desired.

Features across various examples may be utilized in combination or in substitution with each other. Features may be added or excluded as desired. The terms “upper” or “lower” or “side” are relative terms and may be interchanged as desired.

Sensor modules as disclosed herein may comprise bodies (including, e.g., rigid non-conductive frame portions, elastomeric conductive portions, and/or elastomeric non-conductive sealing members) overmolded upon a sensor. In other embodiments, sensor modules may comprise bodies (including, e.g., rigid non-conductive frame portions, elastomeric conductive portions, and/or elastomeric non-conductive sealing members) that are formed separately from the sensor (e.g., in a multi-step overmolding process) and then assembled with a sensor. For example, in some embodiments, an elongate sensor can pierce a molded body to form a sensor module.

The housings as disclosed herein may comprise sensor modules that may be for coupling with a variety of different configurations of on-skin wearable medical devices, testing apparatuses, or other structures. The housings or sensor modules, for example, may be configured for coupling with a first on-skin wearable medical device and also a second on-skin wearable medical device having a different configuration than the first on-skin wearable medical device. Various forms of on-skin wearable medical devices may be accommodated. The housings or sensor modules may be configured for insertion in a socket of a first on-skin wearable medical device and also insertion into a second on-skin wearable medical device, with the second on-skin wearable medical device having a different configuration than the first on-skin wearable medical device. The modular nature of the housings may allow a single form of analyte sensor system to be utilized with a variety of different configurations of devices.

The modular nature of the housings may allow for coupling with a testing apparatus for the elongate sensor. The analyte sensor system, after the overmolding processes, may be coupled to a testing apparatus and then tested. As disclosed herein, improved sealing and protection of the elongate sensor during testing may be provided. The same form of analyte sensor system may then be assembled with a variety of different forms of devices, including a variety of different forms of on-skin wearable medical devices. The housings may protect the elongate sensor during transportation, testing, and assembly. Improved handling (e.g., grasping) may result.

Overmolding processes including non-conductive (e.g., frame and sealing members) and conductive (e.g., electrical contacts) materials may facilitate both mechanical coupling of the elongate sensor to a wearable on-skin medical device and electrical coupling of the elongate sensor to electronics (e.g., a circuit board substrate, sensor electronics, etc.). A small form factor of the housings may allow for ease of integration to several different system architectures and smaller overall sizes of wearable devices. Automated handling of the analyte sensor system may be improved. The use of identifiers (e.g., bar codes) may allow for ease of inspection, testing, and tracking of the analyte sensor systems.

The analyte sensor systems may be placed into trays or packaged in a tape and reel package for reel-to-reel manufacturing in examples.

In examples, the modular nature of the housings may allow for the elongate sensor to be released from a socket or other receiving portion of an on-skin wearable medical device. As such, a user (e.g., a host) may be able to release and remove the analyte sensor system from a socket and replace the analyte sensor system with another analyte sensor system having a different type or configuration of elongate sensor. As such, a user may select the type of elongate sensor used and may swap, replace, and/or choose a type of elongate sensor for use with an on-skin wearable medical device.

Further, the modular nature of the housings may allow for multiple of the analyte sensor systems to be utilized simultaneously with an on-skin wearable medical device. Multiple sockets or other receiving portions of an on-skin wearable medical device may receive multiple of the analyte sensor systems. Multiple elongate sensors may be inserted into the host's skin simultaneously. Different types of elongate sensors may be configured to detect different forms of analytes (e.g., one elongate sensor for detecting glucose and another for detecting iron levels, adrenaline, or other forms of analytes within the host's body). Housings of the analyte sensor systems may be positioned adjacent to each other within a single socket or multiple sockets. Other locations may be utilized in examples. In example, the multiple housings may mate together for use in combination. In examples, multiple elongate sensors may be carried by the same housing.

Features across various examples may be utilized in combination or in substitution with each other. Features may be added or excluded as desired.

The above description presents the best mode contemplated for carrying out the present invention, and of the manner and process of making and using it, in such full, clear, concise, and exact terms as to enable any person skilled in the art to which it pertains to make and use this invention. This invention is, however, susceptible to modifications and alternate constructions from that discussed above that are fully equivalent. Consequently, this invention is not limited to the particular embodiments disclosed. On the contrary, this invention covers all modifications and alternate constructions coming within the spirit and scope of the invention as generally expressed by the following claims, which particularly point out and distinctly claim the subject matter of the invention. While the disclosure has been illustrated and described in detail in the drawings and foregoing description, such illustration and description are to be considered illustrative or exemplary and not restrictive.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article ‘a’ or ‘an’ does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases ‘at least one’ and ‘one or more’ to introduce claim recitations. However, the use of such phrases should not be construed to imply that the introduction of a claim recitation by the indefinite articles ‘a’ or ‘an’ limits any particular claim containing such introduced claim recitation to embodiments containing only one such recitation, even when the same claim includes the introductory phrases ‘one or more’ or ‘at least one’ and indefinite articles such as ‘a’ or ‘an’ (e.g., ‘a’ and/or ‘an’ should typically be interpreted to mean ‘at least one’ or ‘one or more’); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of ‘two recitations,’ without other modifiers, typically means at least two recitations, or two or more recitations). Furthermore, in those instances where a convention analogous to ‘at least one of A, B, and C, etc.’ is used, in general such a construction is intended in the sense one having skill in the art would understand the convention (e.g., ‘a system having at least one of A, B, and C’ would include but not be limited to systems that have A alone, B alone, C alone, A and B together, A and C together, B and C together, and/or A, B, and C together, etc.). In those instances where a convention analogous to ‘at least one of A, B, or C, etc.’ is used, in general such a construction is intended in the sense one having skill in the art would understand the convention (e.g., ‘a system having at least one of A, B, or C’ would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase ‘A or B’ will be understood to include the possibilities of ‘A’ or ‘B’ or ‘A and B.’

All numbers expressing quantities of ingredients, reaction conditions, and so forth used in the specification are to be understood as being modified in all instances by the term ‘about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

1. A sensor module comprising:
an elongate sensor having a distal end portion and a proximal end portion, the distal end portion configured to be inserted into skin of a host and the proximal end portion configured to be positioned exterior of the skin of the host; and
a body molded upon at least a portion of the elongate sensor.

2. The sensor module of claim 1, wherein the elongate sensor includes an outer surface, and the body forms a seal upon at least a portion of the outer surface of the elongate sensor.

3. The sensor module of claim 1, wherein the body comprises a multi-shot overmolding upon at least the portion of the elongate sensor.

4. The sensor module of claim 1, wherein the body comprises a frame disposed at the proximal end portion of the elongate sensor.

5. The sensor module of claim 4, wherein the proximal end portion of the elongate sensor includes one or more sensor electrical contacts, the one or more sensor electrical contacts being positioned within an interior chamber of the frame.

6. The sensor module of claim 5, wherein the frame includes one or more openings configured to allow for electrical connection of the one or more sensor electrical contacts with one or more electrical contacts of a sensor electronics module.

7. The sensor module of claim 6, wherein the frame includes an upper outer surface and a lower outer surface facing opposite the upper outer surface, the upper outer surface and the lower outer surface each extending along a length of the proximal end portion of the elongate sensor, and the one or more openings being positioned in the upper outer surface or the lower outer surface.

8. The sensor module of claim 5, wherein the one or more sensor electrical contacts include a first sensor electrical contact and a second sensor electrical contact, and the interior chamber is a first interior chamber, and the frame includes a second interior chamber, and the first sensor electrical contact is positioned within the first interior chamber, and the second sensor electrical contact is positioned within the second interior chamber.

9.-13. (canceled)

14. The sensor module of claim 4, wherein the frame includes at least one mating feature configured to mate the frame with a corresponding structure of a wearable device.

15. The sensor module of claim 4, wherein the body comprises one or more electrical contacts, with the frame comprising a first material and the one or more electrical contacts of the body comprising a second material different from the first material.

16. (canceled)

17. The sensor module of claim 4, wherein the frame comprises a rigid thermoplastic material.

18. (canceled)

19. The sensor module of claim 1, wherein the body comprises one or more electrical contacts, and wherein the one or more electrical contacts of the body comprise a conductive elastomeric material.

20. (canceled)

21. The sensor module of claim 1, wherein the body comprises one or more electrical contacts, and wherein the one or more electrical contacts of the body are configured for a surface-to-surface connection with one or more electrical contacts of a sensor electronics module.

22. The sensor module of claim 1, wherein the body comprises one or more electrical contacts, and wherein the one or more electrical contacts of the body are configured to be penetrated by one or more electrical contacts of a sensor electronics module.

23. (canceled)

24. The sensor module of claim 1, wherein the body comprises one or more electrical contacts, and wherein the body includes a sealing member configured to seal an electrical connection between the one or more electrical contacts of the body and one or more electrical contacts of a sensor electronics module.

25. (canceled)

26. The sensor module of claim 24, wherein the sealing member covers the one or more electrical contacts of the body and is configured to be penetrated by one or more external electrical contacts of a sensor electronics module.

27. The sensor module of claim 24, wherein the sealing member forms an outer surface of the body, and the one or more electrical contacts of the body protrude from the outer surface.

28.-32. (canceled)

33. The sensor module of claim 1, wherein the elongate sensor comprises a coaxial wire.

34.-37. (canceled)

38. The sensor module of claim 1, wherein the elongate sensor comprises a planar sensor.

39.-55. (canceled)

56. The sensor module of claim 1, wherein the body is physically bonded to at least the portion of the elongate sensor.

57.-163. (canceled)
